(19) United States
(12) Patent Application Publication
(10) Pub. No.: US 2025/0084091 A1
(43) Pub. Date: Mar. 13, 2025

(54) INHIBITORS OF SARS-COV-2

(71) Applicant: The Texas A&M University System, College Station, TX (US)

(72) Inventors: James C. Sacchettini, College Station, TX (US); Nian E. Zhou, Naperville, IL (US); Maloy Kumar Parai, College Station, TX (US); Joonyoung Shin, College Station, TX (US); Jeremy Lee Wood, College Station, TX (US); Inna V. Krieger, College Station, TX (US); Armando Flores, College Station, TX (US); Arjun Acharya, Florence, SC (US); Zhe Shi, Germantown, TN (US); Xuelin Bian, College Station, TX (US); Su TANG, College Station, TX (US); Radha Bam, College Station, TX (US); Pradeep Kumar Jaiswal, College Station, TX (US)

(21) Appl. No.: 18/558,908

(22) PCT Filed: May 4, 2022
(86) PCT No.: PCT/US2022/027690

§ 371 Date: Nov. 3, 2023

Related U.S. Application Data
(60) Provisional application No. 63/183,642, filed on May 4, 2021.
(60) Provisional application No. 63/292,844, filed on Dec. 22, 2021.

(51) Int. Cl. C07D 487/08; A61K 31/444; A61K 31/4545; A61K 31/496; A61K 31/4995; A61K 31/55; A61K 45/06; C07D 213/82; C07D 409/12; C07D 409/14; C07D 471/04; C07D 519/00

(52) U.S. Cl. CPC C07D 487/08; A61K 31/444; A61K 31/4545; A61K 31/496; A61K 31/4995; A61K 31/55; A61K 45/06; C07D 213/82; C07D 409/12; C07D 409/14; C07D 471/04; C07D 519/00

(57) ABSTRACT

Described herein are compounds and methods for the treatment of coronavirus infection. The compounds can function as an inhibitor of the main protease (Mpro) of coronaviruses. The compounds can include diphenylmethyl piperazine derivatives, diphenylmethyl piperidine derivatives, diphenylmethylidene piperidine derivatives, tricyclo[9.4.0.03,8]pentadeca-1(11),3(8),4,6,12,14-hexaen derivatives, tricyclo[9.4.0.03,8]pentadeca-1(11),3(8),4,6,9,12,14-heptaen derivatives, 6,11-dihydrobenzo[c][1]benzoxepin derivatives, 6,11-dihydrobenzo[c][1]benzothiepin derivatives, 5,5-dioxo-6,11-dihydrobenzo[c][1]benzothiepin derivatives, and 6-oxo-5,11-dihydrobenzo[c][1]benzazepin derivatives, as well as pharmaceutically acceptable salts, hydrates, and prodrugs thereof.

### CROSS-REFERENCE TO RELATED APPLICATIONS

The application claims the benefit of U.S. Provisional Application No. 63/183,642, filed May 4, 2021, and U.S. Provisional Application No. 63/292,844, filed Dec. 22, 2021, which are hereby incorporated herein by reference in their entirety.

### BACKGROUND

Novel coronaviruses (CoVs) belong to the zoonotic Coronaviridae family, which cause acute respiratory diseases. Coronaviruses are positive-strand RNA viruses that translate their first open reading frames into polyproteins that are processed by viral proteases into active components.

The world has seen extremely virulent strains of these viruses during the Severe Acute Respiratory Syndrome (SARS) outbreak of 2002, Middle East Respiratory Syndrome (MERS) outbreak of 2012, and during the current novel coronavirus disease (COVID-19) outbreak. Once transmitted from animals to humans, CoV strains can spread rapidly within a community through multiple routes of exposure. The virus that causes COVID-19, i.e., SARS-CoV-2, has high level of infectivity.

In humans, coronaviruses cause respiratory tract infections that can range from mild to lethal. Mild illnesses include some cases of the common cold (which has other possible causes, predominantly rhinoviruses), while more lethal varieties can cause severe acute respiratory syndrome (SARS), Middle East respiratory syndrome (MERS), and coronavirus 2019 (COVID-2019). Symptoms in other species vary; in chickens, they cause an upper respiratory tract disease, while in cows and pigs they cause diarrhea.

Human coronaviruses vary significantly in risk factor. Some can kill more than 30% of those infected (such as MERS-CoV), and some are relatively harmless, such as the common cold. Coronaviruses cause colds with major symptoms, such as fever, and a sore throat from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). The human coronavirus discovered in 2003, SARS-CoV, which causes severe acute respiratory syndrome (SARS), has a unique pathogenesis because it causes both upper and lower respiratory tract infections.

Coronavirus disease 2019 (COVID-19) is an infectious disease caused by severe acute respiratory syndrome coronavirus 2 (SARS-CoV-2). The disease was first identified in December 2019 in Wuhan, the capital of China's Hubei province, and has since spread globally, resulting in the ongoing 2019-20 coronavirus pandemic. Common symptoms include fever, cough, and shortness of breath. Other symptoms may include fatigue, muscle pain, diarrhea, sore throat, loss of smell, and abdominal pain. The time from exposure to onset of symptoms is typically around five days but may range from two to fourteen days. While the majority of cases result in mild symptoms, some progress to viral pneumonia and multi-organ failure.

More than 150 million people have contracted severe acute respiratory syndrome coronavirus-2 (SARS-CoV-2, also referred to as SCV-2 hereafter) worldwide, resulting in over 3.2 million deaths as of Apr. 22, 2021. A safe and efficacious vaccine for SCV-2 will be essential to control the diseases; however, despite enormous global efforts we still lack knowledge of effective vaccines for SCV-2. Even if successful, immunity from the vaccines may be finite and immunity duration remains uncertain.

Therefore, effective antivirals for SCV-2 are needed, not only to treat and/or prevent acute infections but also to decrease the shedding of virus from infected patients.

### SUMMARY

Described herein are compounds including diphenylmethyl piperazine derivatives, diphenylmethyl piperidine derivatives, diphenylmethylidene piperidine derivatives, tricyclo[9.4.0.03,8]pentadeca-1(11),3(8),4,6,12,14-hexaen derivatives, tricyclo[9.4.0.03,8]pentadeca-1(11),3(8),4,6,9,12,14-heptaen derivatives, 6,11-dihydrobenzo[c][1]benzoxepin derivatives, 6,11-dihydrobenzo[c][1]benzothiepin derivatives, 5,5-dioxo-6,11-dihydrobenzo[c][1]benzothiepin derivatives, and 6-oxo-5,11-dihydrobenzo[c][1]benzazepin derivatives, as well as pharmaceutically acceptable salts, hydrates, and prodrugs thereof.

The compounds can function as an inhibitor of the main protease (Mpro) of coronaviruses. In some embodiments, the compounds can function as an inhibitor of the main protease (Mpro) of an alphacoronavirus, a betacoronavirus, a gammacoronavirus, or a deltacoronavirus. In some embodiments, the compounds can function as an inhibitor the main protease (Mpro) of an avian coronavirus (IBV), porcine coronavirus HKU15 (PorCoV HKU15), Porcine epidemic diarrhea virus (PEDV), HCoV-229E, HCoV—OC43, HCoV—HKU1, HCoV-NL63, SARS-CoV, SARS-CoV-2, or MERS-CoV. In certain embodiments, the compounds can function as an inhibitor the main protease (Mpro) of SARS-CoV-2. As a consequence, the compounds described herein can be used to treat and/or prevent a coronavirus infection. In certain embodiments, the compounds described herein can be used to treat and/or prevent SARS-CoV-2 infection (i.e., Covid-19).

The foregoing has outlined rather broadly the features of the present disclosure in order that the detailed description that follows may be better understood. Additional features and advantages of the disclosure will be described hereinafter, which form the subject of the claims.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

### General Definitions

To facilitate understanding of the disclosure set forth herein, a number of terms are defined below. Unless defined otherwise, all technical and scientific terms used herein have the same meanings as commonly understood by one of skill in the art to which the disclosed invention belongs. Publications cited herein and the materials for which they are cited are specifically incorporated by reference.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein. It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrastemal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, hamster, ferret, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety.

The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

### Stereoisomers and Polymorphic Forms

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The compounds described herein can exist and be isolated as optically active and racemic forms. The compounds can have one or more chiral centers, including at a sulfur atom, and thus exist as one or more stereoisomers. Where compounds include n chiral centers, the compounds can comprise up to 2ⁿ optical isomers. Such stereoisomer-containing compounds can exist as a single enantiomer, a mixture of enantiomers, a mixture of diastereomers, or a racemic mixture. The optically active forms can be prepared by, for example, resolution of the racemic forms by selective crystallization techniques, by synthesis from optically active precursors, by chiral synthesis, by chromatographic separation using a chiral stationary phase or by enzymatic resolution.

The compounds can also be present in different solid forms, including different crystalline forms (i.e., different crystalline polymorphs of the compounds) or as an amorphous solid. In addition, the compounds can exist as hydrates or solvates, in which a certain stoichiometric amount of water or a solvent is associated with the molecule in the crystalline form. In some embodiments, the compositions described herein can include up to 15% (w/w), up to 20% (w/w), or up to 30% (w/w) of a particular solid form of the compounds described herein, based on the total weight of the composition.

### Pharmaceutically Acceptable Salts

The compounds described herein can also be provided as pharmaceutically acceptable salts (e.g., acid or base salts) where applicable, of the compounds described herein. Pharmaceutically acceptable salts are known in the art. See, for example, Remington's Pharmaceutical Sciences, 20th ed., Lippincott Williams & Wilkins, Baltimore, MD, 2000, p. 704.

The term “acid salt” contemplates salts of the compounds with all pharmaceutically acceptable inorganic or organic acids. Inorganic acids include mineral acids such as hydrohalic acids such as hydrobromic acid and hydrochloric acid, sulfuric acid, phosphoric acids and nitric acid. Organic acids include all pharmaceutically acceptable aliphatic, alicyclic and aromatic carboxylic acids, dicarboxylic acids, tricarboxylic acids and fatty acids. In one embodiment of the acids, the acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, acetic acid, propionic acid, isopropionic acid, valeric acid, α-hydroxy acids such as glycolic acid and lactic acid, chloroacetic acid, benzoic acid, methane sulfonic acid, and salicylic acid. Examples of dicarboxylic acids include oxalic acid, malic acid, succinic acid, tartaric acid, fumaric acid, and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

The term “base salt” contemplates salts of the compounds with all pharmaceutically acceptable inorganic or organic bases, including hydroxides, carbonates or bicarbonates of alkali metal or alkaline earth metals. Salts formed with such bases include, for example, the alkali metal and alkaline earth metal salts, including, but not limited to, as the lithium, sodium, potassium, magnesium or calcium salts. Salts formed with organic bases include the common hydrocarbon and heterocyclic amine salts, which include, for example, ammonium salts (NH₄⁺), alkylammonium salts, and dialkylammonium salts, as well as salts of cyclic amines such as the morpholine and piperidine salts.

### Prodrugs

The compounds described herein can also be provided as pharmaceutically acceptable prodrugs. Prodrugs of are compounds that, when metabolized in vivo, undergo conversion to compounds described herein having the desired pharmacological activity. Prodrugs can be prepared by replacing appropriate functionalities present in the compounds described herein with “pro-moieties” as described, for example, in H. Bundgaar, Design of Prodrugs (1985). Examples of prodrugs include ester, ether or amide derivatives of the compounds described herein, as well as their pharmaceutically acceptable salts. For further discussions of prodrugs, see, for example, T. Higuchi and V. Stella “Pro-drugs as Novel Delivery Systems,” ACS Symposium Series 14 (1975) and E. B. Roche ed., Bioreversible Carriers in Drug Design (1987).

Reference will now be made in detail to specific aspects of the disclosed materials, compounds, compositions, articles, and methods, examples of which are illustrated in the accompanying Examples and Figures.

### Compounds

Described herein are compounds including diphenylmethyl piperazine derivatives, diphenylmethyl piperidine derivatives, diphenylmethylidene piperidine derivatives, tricyclo[9.4.0.03,8]pentadeca-1(11),3(8),4,6,12,14-hexaen derivatives, tricyclo[9.4.0.03,8]pentadeca-1(11),3(8),4,6,9,12,14-heptaen derivatives, 6,11-dihydrobenzo[c][1]benzoxepin derivatives, 6,11-dihydrobenzo[c][1]benzothiepin derivatives, 5,5-dioxo-6,11-dihydrobenzo[c][1]benzothiepin derivatives, and 6-oxo-5,11-dihydrobenzo[c][1]benzazepin derivatives, as well as pharmaceutically acceptable salts, hydrates, and prodrugs thereof.

The compounds can function as an inhibitor of the main protease (Mpro) of coronaviruses. In some embodiments, the compounds can function as an inhibitor of the main protease (Mpro) of an alphacoronavirus, a betacoronavirus, a gammacoronavirus, or a deltacoronavirus. In some embodiments, the compounds can function as an inhibitor the main protease (Mpro) of an avian coronavirus (IBV), porcine coronavirus HKU15 (PorCoV HKU15), Porcine epidemic diarrhea virus (PEDV), HCoV-229E, HCoV—OC43, HCoV—HKU1, HCoV-NL63, SARS-CoV, SARS-CoV-2, or MERS-CoV. In certain embodiments, the compounds can function as an inhibitor the main protease (Mpro) of SARS-CoV-2. As a consequence, the compounds described herein can be used to treat and/or prevent a coronavirus infection. In certain embodiments, the compounds described herein can be used to treat and/or prevent SARS-CoV-2 infection (i.e., Covid-19).

For example, described herein are diphenylmethyl piperazine derivatives, diphenylmethyl piperidine derivatives and diphenylmethylidene piperidine derivatives defined by Formula (I) below

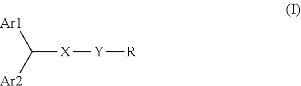

or a pharmaceutically acceptable salt or prodrug thereof, wherein
Ar1 and Ar2 are independently selected from aryl or heteroaryl, each optionally substituted with one or more substituents individually chosen from R⁵;
X is chosen from the following groups:

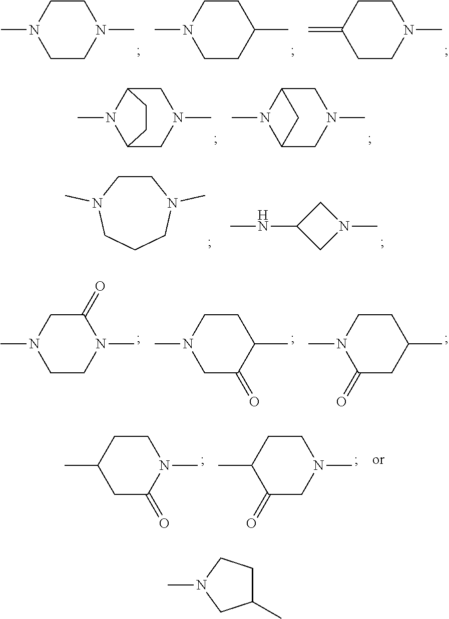

Y is selected from —CO—, —SO₂—, —S(═O)—, —S(O)₂NR⁶—, —S(═O)(═NR⁶)—, and —CH₂—;
R is selected from aryl, heteroaryl, cycloalkyl, and cycloheteroalkyl, each optionally substituted with one or more substituents individually chosen from R⁵;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

In some embodiments of Formula (I), Ar1 and Ar2 are each independently a 5-7 membered aryl or heteroaryl group. In some cases, Ar1 and Ar2 are each independently chosen from phenyl, pyridine, thiophene, furan, pyrrole, imidazole, pyrimidine, pyrazine, indole, benzothiophene, benzofuran, benzoxazole, benzothiozole and benzimidazole. In certain cases, Ar1 and Ar2 are each independently chosen from phenyl and pyridine. In certain cases, Ar1 and Ar2 are each phenyl.

In some embodiments, Ar1 and A2 are each optionally substituted with from one to four substituents (e.g., from one to three substituents) individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments, Ar1 and A2 are each phenyl substituted with from one to three substituents (e.g., from one to two substituents) individually chosen from R⁵. In some embodiments, Ar1 and A2 are each phenyl substituted with one substituent individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In other embodiments, Ar1 and A2 are each unsubstituted phenyl.

In some embodiments, Ar1 is unsubstituted phenyl and A2 is phenyl substituted with from one to three substituents (e.g., from one to two substituents) individually chosen from R⁵. In some embodiments, Ar1 is unsubstituted phenyl and A2 is phenyl substituted with one substituent chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments of Formula (I), X is chosen from the following groups:

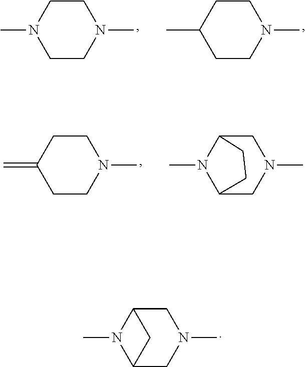

In some embodiments of Formula (I), X is chosen from the following groups:

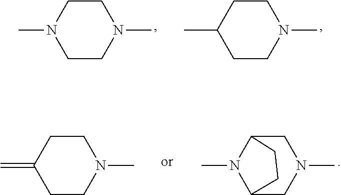

In certain embodiments of Formula (I), X is

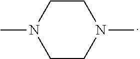

In certain embodiments of Formula (I), X is

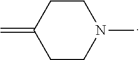

In some embodiments of Formula (I), Y is selected from —CO—, —SO₂—, and —CH₂—.

In some embodiments of Formula (I), Y is —CH₂—. In other embodiments, Y is —CO—. In other embodiments, Y is —SO₂—.

In some embodiments of Formula (I), R is a 5-10 membered ring (e.g., a 5-7 membered ring). In some embodiments, R is a 5-7 membered aryl or heteroaryl group. In some embodiments R is chosen from a phenyl, pyridine, thiophen, furan, pyrrole, imidazole, thiazole, oxazole, pyrimidine, pyrazine, indole, benzothiophene, benzofuran, benzoxazole, benzothiozole, benzimidazole, piperazine, piperidine, morpholine, quinuclidine, pyrrolo-pyridine, imidazo-pyridine, pyrazolo-pyridine, furo-pyridine and thieno-pyridine, or pyrrolidine group. In certain embodiments, R is chosen from pyrrolo-pyridine, imidazo-pyridine, pyrazolo-pyridine, furo-pyridine, or thieno-pyridine. In certain embodiments, R is a pyrrlo-pyridine or azaindole group.

In some embodiments, R is optionally substituted with from one to four substituents (e.g., from one to three substituents) individually chosen from R⁵. In certain embodiments, R⁵ can be chosen from halogen, hydroxyl, alkyl, haloalkyl, alkoxy, nitrile, nitro, amino, alkylamino, dialkylamino, carboxy, acyl, carboxamido, alkylthiol, alkylsulfoxide, alkylsulfonyl, and acylamino groups.

Also described are diphenyl hydroxymethyl piperidine derivatives defined by Formula (II) below

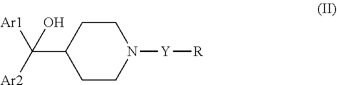

or a pharmaceutically acceptable salt or prodrug thereof, wherein, as valence and stability permit,
Ar1 and Ar2 are independently selected from aryl or heteroaryl, each optionally substituted with one or more substituents individually chosen from R⁵;
Y is selected from —CO—, —SO₂—, —S(═O)—, —S(O)₂NR⁶—, —S(═O)(═NR⁶)—, and —CH₂—;
R is selected from aryl, heteroaryl, cycloalkyl, and cycloheteroalkyl, each optionally substituted with one or more substituents individually chosen from R⁵;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

In some embodiments of Formula (II), Ar1 and Ar2 are each independently a 5-7 membered aryl or heteroaryl group. In some cases, Ar1 and Ar2 are each independently chosen from phenyl, pyridine, thiophene, furan, pyrrole, imidazole, pyrimidine, pyrazine, indole, benzothiophene, benzofuran, benzoxazole, benzothiozole and benzimidazole. In certain cases, Ar1 and Ar2 are each independently chosen from phenyl and pyridine. In certain cases, Ar1 and Ar2 are each phenyl.

In some embodiments, Ar1 and A2 are each optionally substituted with from one to four substituents (e.g., from one to three substituents) individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments, Ar1 and A2 are each phenyl substituted with from one to three substituents (e.g., from one to two substituents) individually chosen from R⁵. In some embodiments, Ar1 and A2 are each phenyl substituted with one substituent individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In other embodiments, Ar1 and A2 are each unsubstituted phenyl.

In some embodiments, Ar1 is unsubstituted phenyl and A2 is phenyl substituted with from one to three substituents (e.g., from one to two substituents) individually chosen from R⁵. In some embodiments, Ar1 is unsubstituted phenyl and A2 is phenyl substituted with one substituent chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments of Formula (II), Y is selected from —CO—, —SO₂—, and —CH₂—.

In some embodiments of Formula (II), Y is —CH₂—. In other embodiments, Y is —CO—. In other embodiments, Y is —SO₂—.

In some embodiments of Formula (II), R is a 5-10 membered ring (e.g., a 5-7 membered ring). In some embodiments, R is a 5-7 membered aryl or heteroaryl group. In some embodiments, R is chosen from a phenyl, pyridine, thiophen, furan, pyrrole, imidazole, thiazole, oxazole, pyrimidine, pyrazine, indole, benzothiophene, benzofuran, benzoxazole, benzothiozole, benzimidazole, piperazine, piperidine, morpholine, quinuclidine, pyrrolo-pyridine, imidazo-pyridine, pyrazolo-pyridine, furo-pyridine and thieno-pyridine, or pyrrolidine group. In certain embodiments, R is chosen from pyrrolo-pyridine, imidazo-pyridine, pyrazolo-pyridine, furo-pyridine, or thieno-pyridine. In certain embodiments, R is a pyrrlo-pyridine or azaindole group.

In some embodiments, R is optionally substituted with from one to four substituents (e.g., from one to three substituents) individually chosen from R⁵. In certain embodiments, R⁵ can be chosen from halogen, hydroxyl, alkyl, haloalkyl, alkoxy, nitrile, nitro, amino, alkylamino, dialkylamino, carboxy, acyl, carboxamido, alkylthiol, alkylsulfoxide, alkylsulfonyl, and acylamino groups.

Also described are tricyclo[9.4.0.03,8]pentadeca-1(11),3(8),4,6,12,14-hexaen derivatives defined by Formula (III) below, tricyclo[9.4.0.03,8]pentadeca-1(11),3(8),4,6,9,12,14-heptaen derivatives defined by Formula (IV) below, 6,11-dihydrobenzo[c][1]benzoxepin derivatives defined by Formula (V) below, 6,11-dihydrobenzo[c][1]benzothiepin derivatives defined by Formula (VI) below, 5,5-dioxo-6,11-dihydrobenzo[c][1]benzothiepin derivatives defined by Formula (VII) below, and 6-oxo-5,11-dihydrobenzo[c][1]benzazepin derivatives defined by Formula (VIII) below
and compounds defined by Formula (IX)—(XI) below.

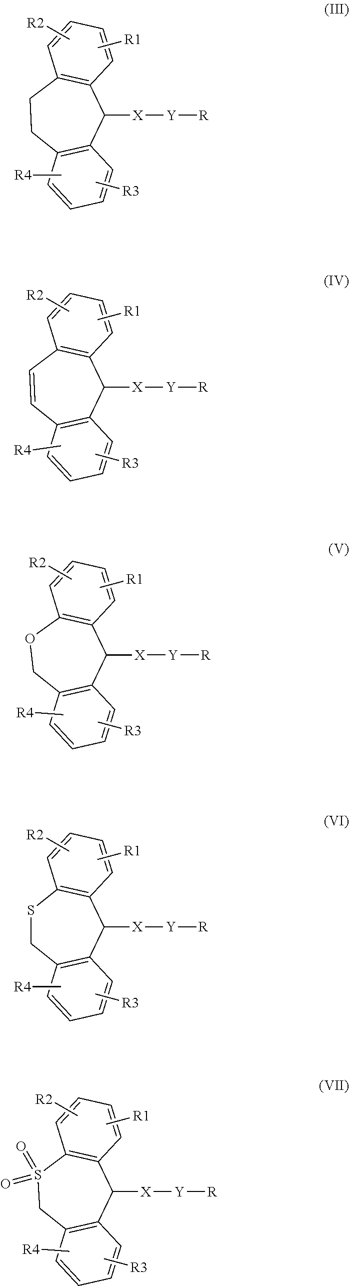

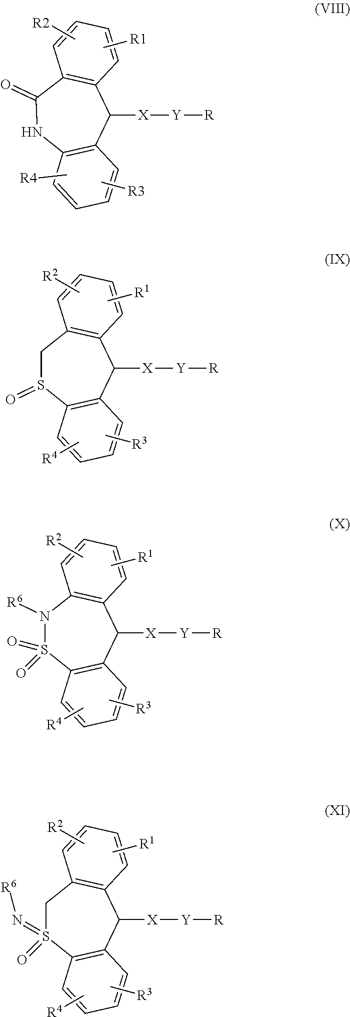

or pharmaceutically acceptable salts or prodrugs thereof, wherein, as valence and stability permit,
X is chosen from the following groups:

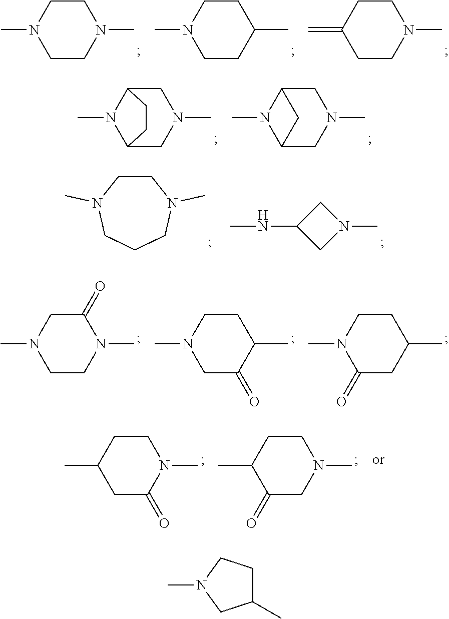

Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵;
R¹, R², R³, and R⁴ are each individually chosen from hydrogen, hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

In some embodiments of Formula (III)—(XI), X is chosen from the following groups:

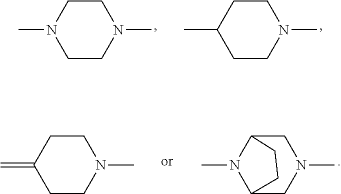

In some embodiments of Formula (III)—(XI), X is

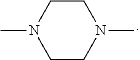

In some embodiments of Formula (III)—(XI), X is

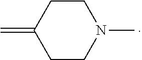

In some embodiments of Formula (III)—(XI), X is

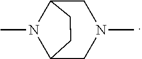

In some embodiments of Formula (III)—(XI), Y is selected from —CO—, —SO₂—, and —CH₂—.

In some embodiments of Formula (III)—(XI), Y is —CH₂—. In other embodiments, Y is —CO—. In other embodiments, Y is —SO₂—.

In some embodiments of Formula (III)—(XI), R is a 5-10 membered ring (e.g., a 5-7 membered ring). In some embodiments, R is a 5-7 membered aryl or heteroaryl group. In some embodiments, R is chosen from a phenyl, pyridine, thiophen, furan, pyrrole, imidazole, thiazole, oxazole, pyrimidine, pyrazine, indole, benzothiophene, benzofuran, benzoxazole, benzothiozole, benzimidazole, piperazine, piperidine, morpholine, quinuclidine, pyrrolo-pyridine, imidazo-pyridine, pyrazolo-pyridine, furo-pyridine and thieno-pyridine, or pyrrolidine group. In certain embodiments, R is chosen from pyrrolo-pyridine, imidazo-pyridine, pyrazolo-pyridine, furo-pyridine, or thieno-pyridine. In certain embodiments, R is a pyrrlo-pyridine or azaindole group.

In some embodiments, R is optionally substituted with from one to four substituents (e.g., from one to three substituents) individually chosen from R⁵. In certain embodiments, R⁵ can be chosen from halogen, hydroxyl, alkyl, haloalkyl, alkoxy, nitrile, nitro, amino, alkylamino, dialkylamino, carboxy, acyl, carboxamido, alkylthiol, alkylsulfoxide, alkylsulfonyl, and acylamino groups.

In some embodiments of Formula (III)—(XI), R¹, R², R³, and R⁴ are each individually chosen from hydrogen, halogen, hydroxyl, alkyl, alkoxy, nitrile, nitro, amino, alkylamino, dialkylamino, carboxy, acyl, carboxamido, alkylthiol, alkylsulfoxide, alkylsulfonyl, and acylamino groups.

Also described are compounds described by Formula (XII) below

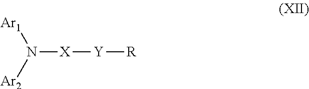

or a pharmaceutically acceptable salt or prodrug thereof, wherein, as valence and stability permit,
Ar1 and Ar2 are independently selected from aryl or heteroaryl, each optionally substituted with one or more substituents individually chosen from R⁵;
X is chosen from the following groups:

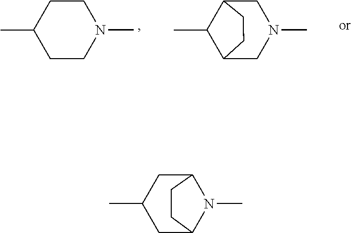

Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

In some embodiments of Formula (XII), Ar1 and Ar2 are each independently a 5-7 membered aryl or heteroaryl group. In some cases, Ar1 and Ar2 are each independently chosen from phenyl, pyridine, thiophene, furan, pyrrole, imidazole, pyrimidine, pyrazine, indole, benzothiophene, benzofuran, benzoxazole, benzothiozole and benzimidazole. In certain cases, Ar1 and Ar2 are each independently chosen from phenyl and pyridine. In certain cases, Ar1 and Ar2 are each phenyl.

In some embodiments, Ar1 and A2 are each optionally substituted with from one to four substituents (e.g., from one to three substituents) individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments, Ar1 and A2 are each phenyl substituted with from one to three substituents (e.g., from one to two substituents) individually chosen from R⁵. In some embodiments, Ar1 and A2 are each phenyl substituted with one substituent individually chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In other embodiments, Ar1 and A2 are each unsubstituted phenyl.

In some embodiments, Ar1 is unsubstituted phenyl and A2 is phenyl substituted with from one to three substituents (e.g., from one to two substituents) individually chosen from R⁵. In some embodiments, Ar1 is unsubstituted phenyl and A2 is phenyl substituted with one substituent chosen from R⁵. In certain embodiments, R⁵ can be chosen from halogen, hydroxyl, haloalkyl, alkyl, alkoxy, nitrile, nitro, amino, alkylamino, dialkylamino, carboxy, acyl, carboxamido, alkylthiol, alkylsulfoxide, alkylsulfonyl, and acylamino groups. In certain embodiments, R⁵ can be halogen (e.g., fluoro).

In some embodiments of Formula (XII), X is

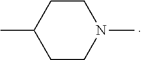

In some embodiments of Formula (XII), Y is selected from —CO—, —SO₂—, and —CH₂—.

In some embodiments of Formula (XII), Y is —CH₂—. In other embodiments, Y is —CO—. In other embodiments, Y is —SO₂—.

In some embodiments of Formula (XII), R is a 5-10 membered ring (e.g., a 5-7 membered ring). In some embodiments, R is a 5-7 membered aryl or heteroaryl group. In some embodiments, R is chosen from a phenyl, pyridine, thiophen, furan, pyrrole, imidazole, thiazole, oxazole, pyrimidine, pyrazine, indole, benzothiophene, benzofuran, benzoxazole, benzothiozole, benzimidazole, piperazine, piperidine, morpholine, quinuclidine, pyrrolo-pyridine, imidazo-pyridine, pyrazolo-pyridine, furo-pyridine and thieno-pyridine, or pyrrolidine group. In certain embodiments, R is chosen from pyrrolo-pyridine, imidazo-pyridine, pyrazolo-pyridine, furo-pyridine, or thieno-pyridine. In certain embodiments, R is a pyrrlo-pyridine or azaindole group.

In some embodiments, R is optionally substituted with from one to four substituents (e.g., from one to three substituents) individually chosen from R⁵. In certain embodiments, R⁵ can be chosen from halogen, hydroxyl, alkyl, haloalkyl, alkoxy, nitrile, nitro, amino, alkylamino, dialkylamino, carboxy, acyl, carboxamido, alkylthiol, alkylsulfoxide, alkylsulfonyl, and acylamino groups.

Example inhibitors include the compounds listed in Table 1 below.

TABLE 1
SARS-COV-2 inhibitors.

| Compound ID | Structure | IUPAC name |
|---|---|---|
| NZ-769 | 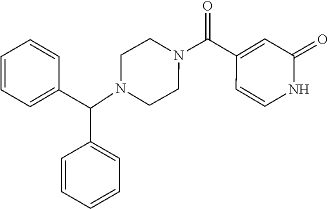 [SMILES: O=C(c1cc[nH]c(=O)c1)N1CCN(C(c2ccccc2)c2ccccc2)CC1] | 4-[4-(diphenylmethyl)piperazine-1- carbonyl]-1,2-dihydropyridin-2-one |
| NZ-770 | 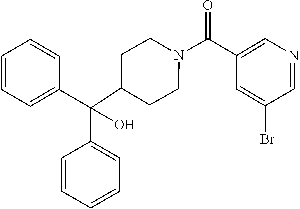 [SMILES: O=C(c1cncc(Br)c1)N1CCC(C(O)(c2ccccc2)c2ccccc2)CC1] | [1-(5-bromopyridine-3- carbonyl)piperidin-4- yl]diphenylmethanol |
| NZ-771 | 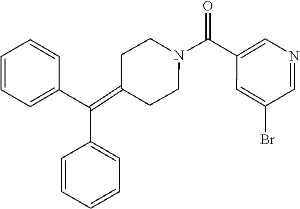 [SMILES: O=C(c1cncc(Br)c1)N1CCC(=C(c2ccccc2)c2ccccc2)CC1] | 3-bromo-5-[4- (diphenylmethylidene)piperidine-1- carbonyl]pyridine |
| NZ-772 | 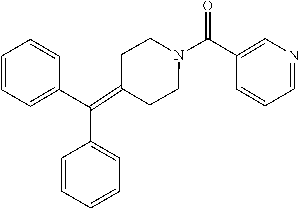 [SMILES: O=C(c1cccnc1)N1CCC(=C(c2ccccc2)c2ccccc2)CC1] | 3-[4-(diphenylmethylidene)piperidine-1- carbonyl]pyridine |
| NZ-773 | 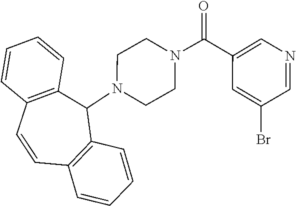 [SMILES: O=C(c1cncc(Br)c1)N1CCN(C2c3ccccc3C=Cc3ccccc32)CC1] | (5-bromo-3-pyridyl)-[4-(2- tricyclo[9.4.0.03,8]pentadeca- 1(11),3(8),4,6,9,12,14- heptaenyl)piperazin-1-yl]methanone |
| NZ-774 | 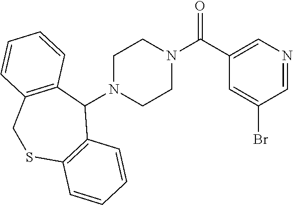 [SMILES: O=C(c1cncc(Br)c1)N1CCN(C2c3ccccc3CSc3ccccc32)CC1] | (5-bromo-3-pyridyl)-[4-(6,11- dihydrobenzo[c][1]benzothiepin-11- yl)piperazin-1-yl]methanone |
| NZ-776 | 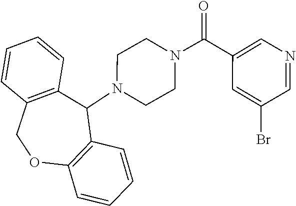 [SMILES: O=C(c1cncc(Br)c1)N1CCN(C2c3ccccc3COc3ccccc32)CC1] | (5-bromo-3-pyridyl)-[4-(6,11- dihydrobenzo[c][1]benzoxepin-11- yl)piperazin-1-yl]methanone |
| NZ-778 | 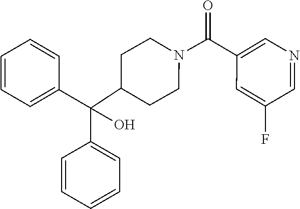 [SMILES: O=C(c1cncc(F)c1)N1CCC(C(O)(c2ccccc2)c2ccccc2)CC1] | [1-(5-fluoropyridine-3- carbonyl)piperidin-4- yl]diphenylmethanol |
| NZ-779 | 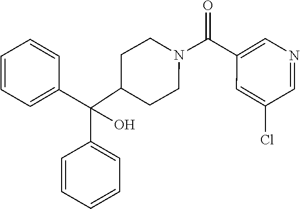 [SMILES: O=C(c1cncc(Cl)c1)N1CCC(C(O)(c2ccccc2)c2ccccc2)CC1] | [1-(5-chloropyridine-3- carbonyl)piperidin-4- yl]diphenylmethanol |
| NZ-780 | 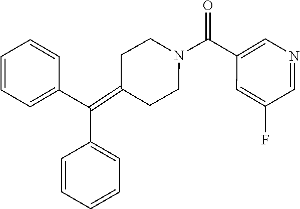 [SMILES: O=C(c1cncc(F)c1)N1CCC(=C(c2ccccc2)c2ccccc2)CC1] | 3-[4-(diphenylmethylidene)piperidine-1- carbonyl]-5-fluoropyridine |
| NZ-781 | 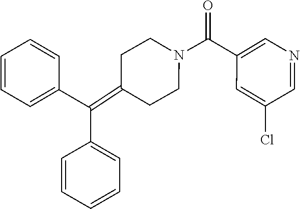 [SMILES: O=C(c1cncc(Cl)c1)N1CCC(=C(c2ccccc2)c2ccccc2)CC1] | 3-chloro-5-[4- (diphenylmethylidene)piperidine-1- carbonyl]pyridine |
| NZ-782 | 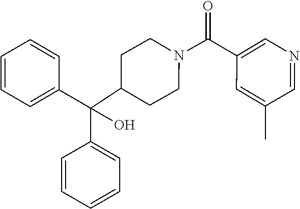 [SMILES: Cc1cncc(C(=O)N2CCC(C(O)(c3ccccc3)c3ccccc3)CC2)c1] | [1-(5-methylpyridine-3- carbonyl)piperidin-4- yl]diphenylmethanol |
| NZ-783 | 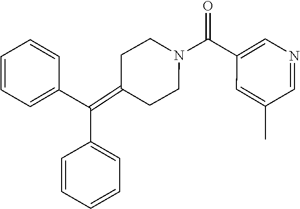 [SMILES: Cc1cncc(C(=O)N2CCC(=C(c3ccccc3)c3ccccc3)CC2)c1] | 3-[4-(diphenylmethylidene)piperidine-1- carbonyl]-5-methylpyridine |
| NZ-784 | 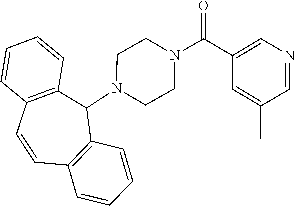 [SMILES: Cc1cncc(C(=O)N2CCN(C3c4ccccc4C=Cc4ccccc43)CC2)c1] | (5-methyl-3-pyridyl)-[4-(2- tricyclo[9.4.0.03,8]pentadeca- 1(11),3(8),4,6,9,12,14- heptaenyl)piperazin-1-yl]methanone |
| NZ-785 | 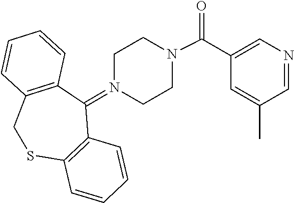 | [4-(6,11- dihydrobenzo[c][1]benzothiepin-11- yl)piperazin-1-yl]-(5-methyl-3- pyridyl)methanone |
| NZ-786 | 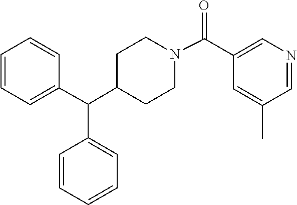 [SMILES: Cc1cncc(C(=O)N2CCC(C(c3ccccc3)c3ccccc3)CC2)c1] | 3-[4-(diphenylmethyl)piperidine-1- carbonyl]-5-methylpyridine |
| NZ-788 | 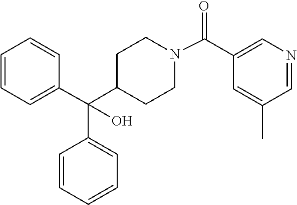 [SMILES: Cc1cncc(C(=O)N2CCC(C(O)(c3ccccc3)c3ccccc3)CC2)c1] | 4-[4- (hydroxydiphenylmethyl)piperidine-1- carbonyl]-2,3-dihydropyridin-2-one |
| NZ-789 | 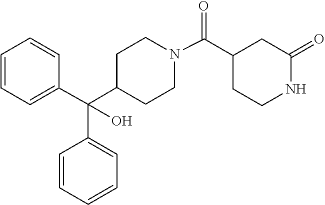 [SMILES: O=C1CC(C(=O)N2CCC(C(O)(c3ccccc3)c3ccccc3)CC2)CCN1] | 4-[4- (hydroxydiphenylmethyl)piperidine-1- carbonyl]piperidin-2-one |
| NZ-790 | 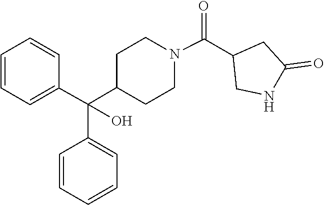 [SMILES: O=C1CC(C(=O)N2CCC(C(O)(c3ccccc3)c3ccccc3)CC2)CN1] | 4-[4- (hydroxydiphenylmethyl)piperidine-1- carbonyl]pyrrolidin-2-one |
| NZ-791 | 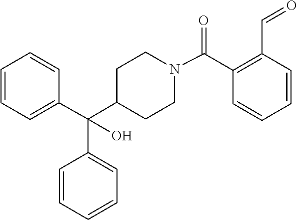 [SMILES: O=Cc1ccccc1C(=O)N1CCC(C(O)(c2ccccc2)c2ccccc2)CC1] | 2-[4- (hydroxydiphenylmethyl)piperidine-1- carbonyl]benzaldehyde |
| NZ-792 | 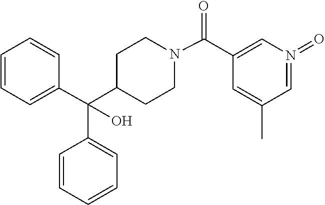 [SMILES: Cc1cc(C(=O)N2CCC(C(O)(c3ccccc3)c3ccccc3)CC2)c[n+]([O-])c1] | [4-[hydroxy(diphenyl)methyl]-1- piperidyl]-(5-methyl-1-oxo-3- pyridyl)methanone |
| NZ-793 | 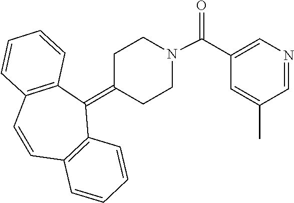 [SMILES: Cc1cncc(C(=O)N2CCC(=C3c4ccccc4C=Cc4ccccc43)CC2)c1] | (5-methyl-3-pyridyl)-[4-(2- tricyclo[9.4.0.03,8]pentadeca- 1(11),3(8),4,6,9,12,14-heptaenylidene)- 1-piperidyl]methanone |
| NZ-794 | 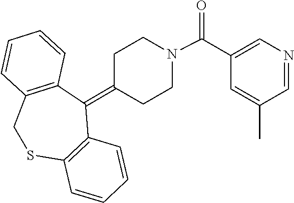 [SMILES: Cc1cncc(C(=O)N2CCC(=C3c4ccccc4CSc4ccccc43)CC2)c1] | [4-(6H-benzo[c][1]benzothiepin-11- ylidene)-1-piperidyl]-(5-methyl-3- pyridyl)methanone |
| NZ-795 | 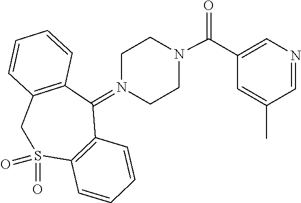 | [4-(5,5-dioxo-6,11- dihydrobenzo[c][1]benzothiepin-11- yl)piperazin-1-yl]-(5-methyl-3- pyridyl)methanone |
| NZ-796 | 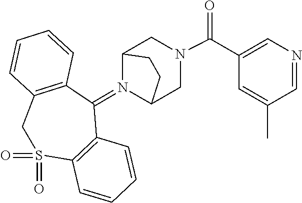 | [8-(5,5-dioxo-6,11- dihydrobenzo[c][1]benzothiepin-11-yl)- 3,8-diazabicyclo[3.2.1]octan-3-yl]-(5- methyl-3-pyridyl)methanone |
| NZ-797 | 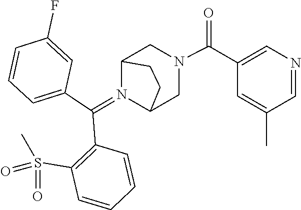 | 8-[(3-fluorophenyl)(2- methanesulfonylphenyl)methyl]-3-(5- methylpyridine-3-carbonyl)-3,8- diazabicyclo[3.2.1]octane |
| NZ-798 | 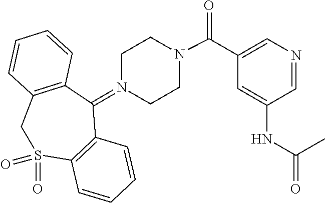 | N-[5-[4-(5,5-dioxo-6,11- dihydrobenzo[c][1]benzothiepin-11- yl)piperazine-1-carbonyl]-3- pyridyl]acetamide |
| NZ-799 | 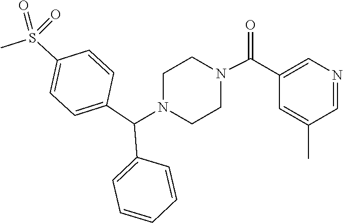 [SMILES: Cc1cncc(C(=O)N2CCN(C(c3ccccc3)c3ccc(S(C)(=O)=O)cc3)CC2)c1] | 1-[(4- methanesulfonylphenyl)(phenyl)methyl]- 4-(5-methylpyridine-3- carbonyl)piperazine |
| NZ-800 | 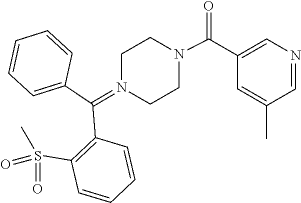 | 1-[(2- methanesulfonylphenyl)(phenyl)methyl]- 4-(5-methylpyridine-3- carbonyl)piperazine |
| NZ-801 | 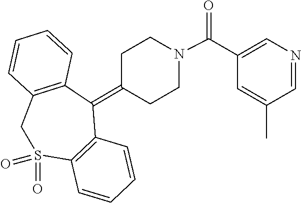 [SMILES: Cc1cncc(C(=O)N2CCC(=C3c4ccccc4CS(=O)(=O)c4ccccc43)CC2)c1] | [4-(5,5-dioxo-6H- benzo[c][1]benzothiepin-11-ylidene)-1- piperidyl]-(5-methyl-3- pyridyl)methanone |
| NZ-802 | 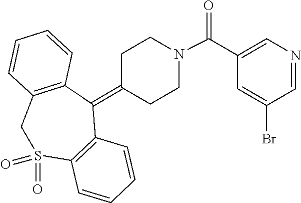 [SMILES: O=C(c1cncc(Br)c1)N1CCC(=C2c3ccccc3CS(=O)(=O)c3ccccc32)CC1] | (5-bromo-3-pyridyl)-[4-(5,5-dioxo-6H- benzo[c][1]benzothiepin-11-ylidene)-1- piperidyl]methanone |
| NZ-803 | 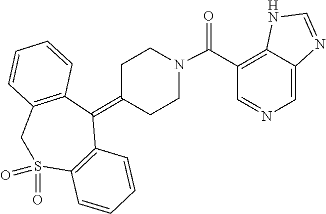 [SMILES: O=C(c1cncc2nc[nH]c12)N1CCC(=C2c3ccccc3CS(=O)(=O)c3ccccc32)CC1] | [4-(5,5-dioxo-6H- benzo[c][1]benzothiepin-11-ylidene)-1- piperidyl]-(1H-imidazo[4,5-c]pyridin-7- yl)methanone |
| NZ-804 | 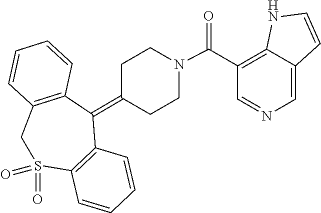 [SMILES: O=C(c1cncc2cc[nH]c12)N1CCC(=C2c3ccccc3CS(=O)(=O)c3ccccc32)CC1] | [4-(5,5-dioxo-6H- benzo[c][1]benzothiepin-11-ylidene)-1- piperidyl]-(1H-pyrrolo[3,2-c]pyridin-7- yl)methanone |
| NZ-805 | 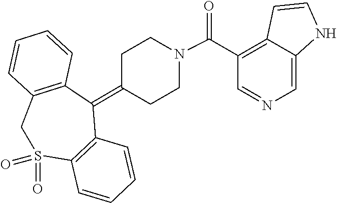 [SMILES: O=C(c1cncc2[nH]ccc12)N1CCC(=C2c3ccccc3CS(=O)(=O)c3ccccc32)CC1] | [4-(5,5-dioxo-6H- benzo[c][1]benzothiepin-11-ylidene)-1- piperidyl]-(1H-pyrrolo[2,3-c]pyridin-4- yl)methanone |
| ZS-312 | 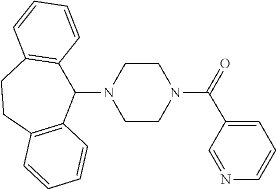 [SMILES: O=C(c1cccnc1)N1CCN(C2c3ccccc3CCc3ccccc32)CC1] | 3-pyridyl-[4-(2- tricyclo[9.4.0.03,8]pentadeca- 1(11),3(8),4,6,12,14- hexaenyl)piperazin-1-yl]methanone |
| ZS-313 | 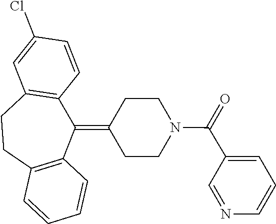 [SMILES: O=C(c1cccnc1)N1CCC(=C2c3ccccc3CCc3cc(Cl)ccc32)CC1] | [4-(13-chloro-4- azatricyclo[9.4.0.03,8]pentadeca- 1(11),3(8),4,6,12,14-hexaen-2-ylidene)- 1-piperidyl]-(3-pyridyl)methanone |
| ZS-316 | 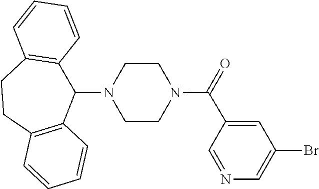 [SMILES: O=C(c1cncc(Br)c1)N1CCN(C2c3ccccc3CCc3ccccc32)CC1] | (5-bromo-3-pyridyl)-[4-(2- tricyclo[9.4.0.03,8]pentadeca- 1(11),3(8),4,6,12,14- hexaenyl)piperazin-1-yl]methanone |
| ZS-317 | 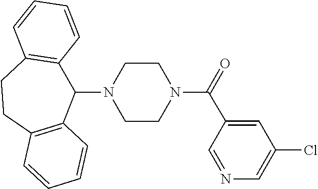 [SMILES: O=C(c1cncc(Cl)c1)N1CCN(C2c3ccccc3CCc3ccccc32)CC1] | (5-chloro-3-pyridyl)-[4-(2- tricyclo[9.4.0.03,8]pentadeca- 1(11),3(8),4,6,12,14- hexaenyl)piperazin-1-yl]methanone |
| ZS-318 | 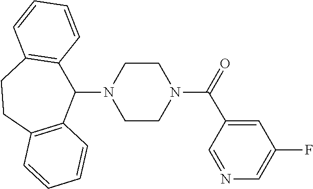 [SMILES: O=C(c1cncc(F)c1)N1CCN(C2c3ccccc3CCc3ccccc32)CC1] | (5-fluoro-3-pyridyl)-[4-(2- tricyclo[9.4.0.03,8]pentadeca- 1(11),3(8),4,6,12,14- hexaenyl)piperazin-1-yl]methanone |
| ZS-319 | 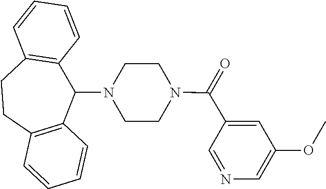 [SMILES: COc1cncc(C(=O)N2CCN(C3c4ccccc4CCc4ccccc43)CC2)c1] | (5-methoxy-3-pyridyl)-[4-(2- tricyclo[9.4.0.03,8]pentadeca- 1(11),3(8),4,6,12,14- hexaenyl)piperazin-1-yl]methanone |
| ZS-320 | 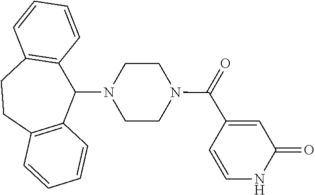 [SMILES: O=C(c1cc[nH]c(=O)c1)N1CCN(C2c3ccccc3CCc3ccccc32)CC1] | 4-[4-(2-tricyclo[9.4.0.03,8]pentadeca- 1(11),3(8),4,6,12,14- hexaenyl)piperazine-1-carbonyl]-1H- pyridin-2-one |
| ZS-324 | 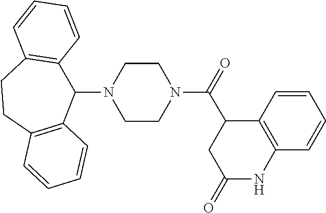 [SMILES: O=C1CC(C(=O)N2CCN(C3c4ccccc4CCc4ccccc43)CC2)c2ccccc2N1] | 4-[4-(2-tricyclo[9.4.0.03,8]pentadeca- 1(11),3(8),4,6,12,14- hexaenyl)piperazine-1-carbonyl]-3,4- dihydro-1H-quinolin-2-one |
| ZS-325 | 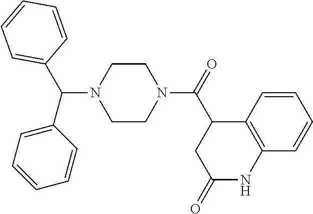 [SMILES: O=C1CC(C(=O)N2CCN(C(c3ccccc3)c3ccccc3)CC2)c2ccccc2N1] | 4-(4-benzhydrylpiperazine-1-carbonyl)- 3,4-dihydro-1H-quinolin-2-one |
| ZS-329 | 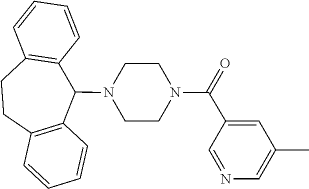 [SMILES: Cc1cncc(C(=O)N2CCN(C3c4ccccc4CCc4ccccc43)CC2)c1] | (5-methyl-3-pyridyl)-[4-(2- tricyclo[9.4.0.03,8]pentadeca- 1(11),3(8),4,6,12,14- hexaenyl)piperazin-1-yl]methanone |
| ZS-331 | 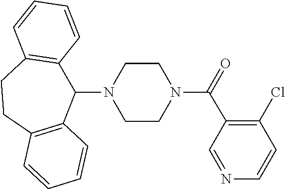 [SMILES: O=C(c1cnccc1Cl)N1CCN(C2c3ccccc3CCc3ccccc32)CC1] | (4-chloro-3-pyridyl)-[4-(2- tricyclo[9.4.0.03,8]pentadeca- 1(11),3(8),4,6,12,14- hexaenyl)piperazin-1-yl]methanone |
| ZS-333 | 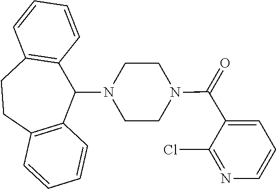 [SMILES: O=C(c1cccnc1Cl)N1CCN(C2c3ccccc3CCc3ccccc32)CC1] | (2-chloro-3-pyridyl)-[4-(2- tricyclo[9.4.0.03,8]pentadeca- 1(11),3(8),4,6,12,14- hexaenyl)piperazin-1-yl]methanone |
| ZS-335 | 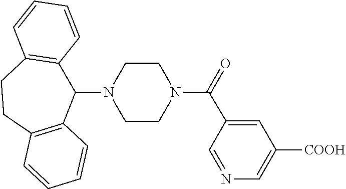 [SMILES: O=C(O)c1cncc(C(=O)N2CCN(C3c4ccccc4CCc4ccccc43)CC2)c1] | 5-[4-(2-tricyclo[9.4.0.03,8]pentadeca- 1(11),3(8),4,6,12,14- hexaenyl)piperazine-1- carbonyl]pyridine-3-carboxylic acid |
| ZS-336 | 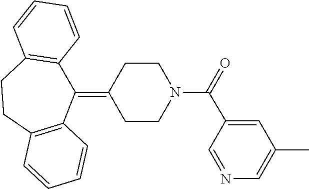 [SMILES: Cc1cncc(C(=O)N2CCC(=C3c4ccccc4CCc4ccccc43)CC2)c1] | (5-methyl-3-pyridyl)-[4-(2- tricyclo[9.4.0.03,8]pentadeca- 1(11),3(8),4,6,12,14-hexaenylidene)-1- piperidyl]methanone |
| ZS-337 | 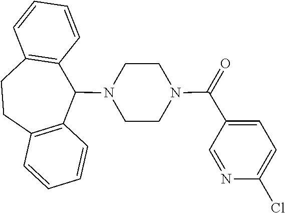 [SMILES: O=C(c1ccc(Cl)nc1)N1CCN(C2c3ccccc3CCc3ccccc32)CC1] | (6-chloro-3-pyridyl)-[4-(2- tricyclo[9.4.0.03,8]pentadeca- 1(11),3(8),4,6,12,14- hexaenyl)piperazin-1-yl]methanone |
| ZS-338 | 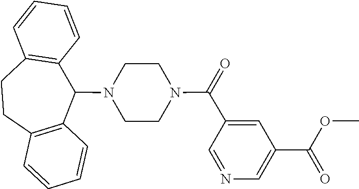 [SMILES: COC(=O)c1cncc(C(=O)N2CCN(C3c4ccccc4CCc4ccccc43)CC2)c1] | methyl 5-[4-(2- tricyclo[9.4.0.03,8]pentadeca- 1(11),3(8),4,6,12,14- hexaenyl)piperazine-1- carbonyl]pyridine-3-carboxylate |
| ZS-339 | 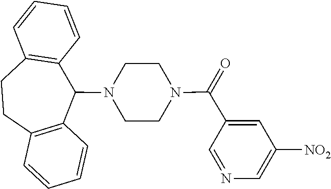 [SMILES: O=C(c1cncc([N+](=O)[O-])c1)N1CCN(C2c3ccccc3CCc3ccccc32)CC1] | (5-nitro-3-pyridyl)-[4-(2- tricyclo[9.4.0.03,8]pentadeca- 1(11),3(8),4,6,12,14- hexaenyl)piperazin-1-yl]methanone |
| ZS-342 | 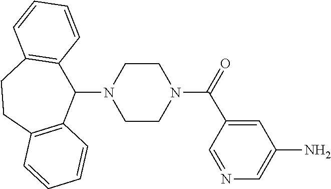 [SMILES: Nc1cncc(C(=O)N2CCN(C3c4ccccc4CCc4ccccc43)CC2)c1] | (5-amino-3-pyridyl)-[4-(2- tricyclo[9.4.0.03,8]pentadeca- 1(11),3(8),4,6,12,14- hexaenyl)piperazin-1-yl]methanone |
| ZS-346 | 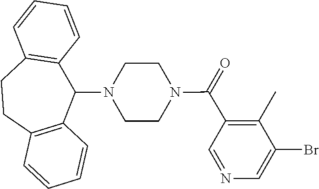 [SMILES: Cc1c(Br)cncc1C(=O)N1CCN(C2c3ccccc3CCc3ccccc32)CC1] | (5-bromo-4-methyl-3-pyridyl)-[4-(2- tricyclo[9.4.0.03,8]pentadeca- 1(11),3(8),4,6,12,14- hexaenyl)piperazin-1-yl]methanone |
| ZS-347 | 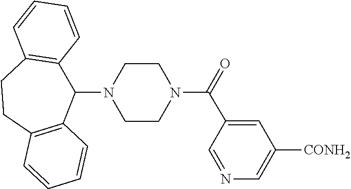 [SMILES: NC(=O)c1cncc(C(=O)N2CCN(C3c4ccccc4CCc4ccccc43)CC2)c1] | 5-[4-(2-tricyclo[9.4.0.03,8]pentadeca- 1(11),3(8),4,6,12,14- hexaenyl)piperazine-1- carbonyl]pyridine-3-carboxamide |
| ZS-355 | 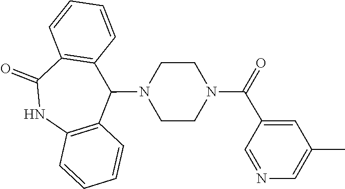 [SMILES: Cc1cncc(C(=O)N2CCN(C3c4ccccc4NC(=O)c4ccccc43)CC2)c1] | 11-[4-(5-methylpyridine-3- carbonyl)piperazin-1-yl]-5,11- dihydrobenzo[c][1]benzazepin-6-one |
| ZS-361 | 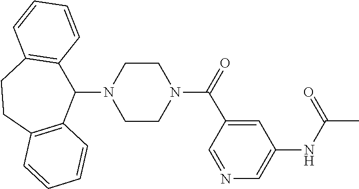 [SMILES: CC(=O)Nc1cncc(C(=O)N2CCN(C3c4ccccc4CCc4ccccc43)CC2)c1] | N-[5-[4-(2- tricyclo[9.4.0.03,8]pentadeca- 1(11),3(8),4,6,12,14- hexaenyl)piperazine-1-carbonyl]-3- pyridyl]acetamide |
| AA-551 | 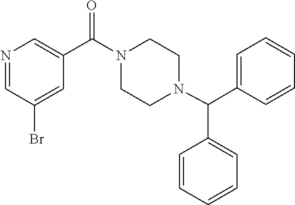 [SMILES: O=C(c1cncc(Br)c1)N1CCN(C(c2ccccc2)c2ccccc2)CC1] | (4-benzhydrylpiperazin-1-yl)(5- bromopyridin-3-yl)methanone |
| AA-557 | 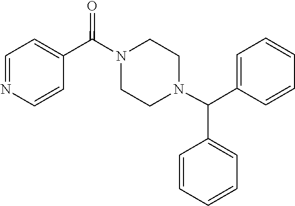 [SMILES: O=C(c1ccncc1)N1CCN(C(c2ccccc2)c2ccccc2)CC1] | (4-benzhydrylpiperazin-1-yl)(pyridin-4- yl)methanone |
| AA-558 | 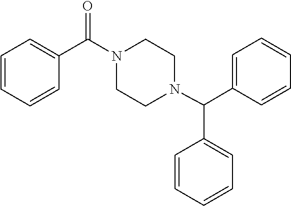 [SMILES: O=C(c1ccccc1)N1CCN(C(c2ccccc2)c2ccccc2)CC1] | (4-benzhydrylpiperazin-1- yl)(phenyl)methanone |
| AA-559 | 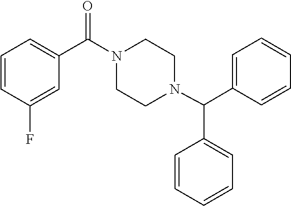 [SMILES: O=C(c1cccc(F)c1)N1CCN(C(c2ccccc2)c2ccccc2)CC1] | (4-benzhydrylpiperazin-1-yl)(3- fluorophenyl)methanone |
| AA-560 | 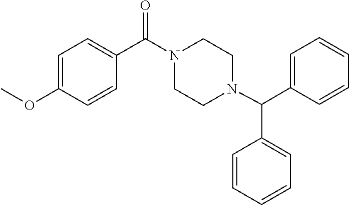 [SMILES: COc1ccc(C(=O)N2CCN(C(c3ccccc3)c3ccccc3)CC2)cc1] | (4-benzhydrylpiperazin-1-yl)(4- methoxyphenyl)methanone |
| AA-564 | 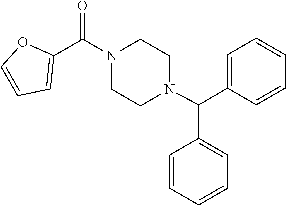 [SMILES: O=C(c1ccco1)N1CCN(C(c2ccccc2)c2ccccc2)CC1] | (4-benzhydrylpiperazin-1-yl)(furan-2- yl)methanone |
| AA-565 | 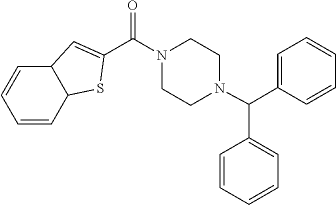 [SMILES: O=C(C1=CC2C=CC=CC2S1)N1CCN(C(c2ccccc2)c2ccccc2)CC1] | (4-benzhydrylpiperazin-1-yl)(3a,7a- dihydrobenzo[b]thiophen-2- yl)methanone |
| AA-566 | 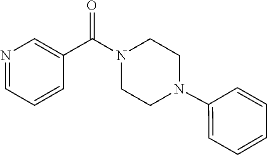 [SMILES: O=C(c1cccnc1)N1CCN(c2ccccc2)CC1] | (4-phenylpiperazin-1-yl)(pyridin-3- yl)methanone |
| AA-567 | 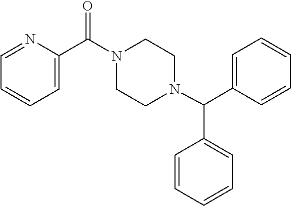 [SMILES: O=C(c1ccccn1)N1CCN(C(c2ccccc2)c2ccccc2)CC1] | (4-benzhydrylpiperazin-1-yl)(pyridin-2- yl)methanone |
| AA-570 | 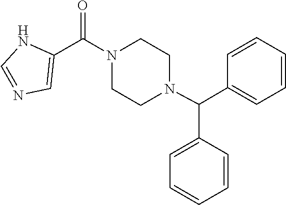 [SMILES: O=C(c1cnc[nH]1)N1CCN(C(c2ccccc2)c2ccccc2)CC1] | (4-benzhydrylpiperazin-1-yl)(1H- imidazol-5-yl)methanone |
| AA-571 | 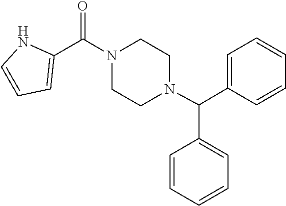 [SMILES: O=C(c1ccc[nH]1)N1CCN(C(c2ccccc2)c2ccccc2)CC1] | (4-benzhydrylpiperazin-1-yl)(1H-pyrrol- 2-yl)methanone |
| AA-572 | 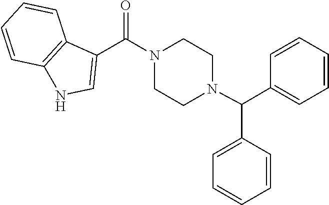 [SMILES: O=C(c1c[nH]c2ccccc12)N1CCN(C(c2ccccc2)c2ccccc2)CC1] | (4-benzhydrylpiperazin-1-yl)(1H-indol- 3-yl)methanone |
| AA-575 | 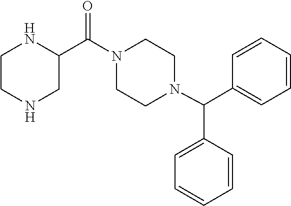 [SMILES: O=C(C1CNCCN1)N1CCN(C(c2ccccc2)c2ccccc2)CC1] | (4-benzhydrylpiperazin-1-yl)(piperazin- 2-yl)methanone |
| AA-577 | 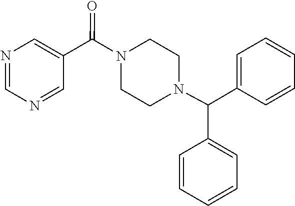 [SMILES: O=C(c1cncnc1)N1CCN(C(c2ccccc2)c2ccccc2)CC1] | (4-benzhydrylpiperazin-1-yl)(pyrimidin- 5-yl)methanone |
| AA-579 | 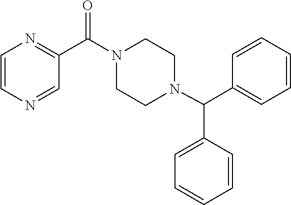 [SMILES: O=C(c1cnccn1)N1CCN(C(c2ccccc2)c2ccccc2)CC1] | (4-benzhydrylpiperazin-1-yl)(pyrazin-2- yl)methanone |
| AA-580 | 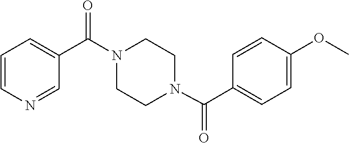 [SMILES: COc1ccc(C(=O)N2CCN(C(=O)c3cccnc3)CC2)cc1] | (4-(4-methoxybenzoyl)piperazin-1- yl)(pyridin-3-yl)methanone |
| AA-581 | 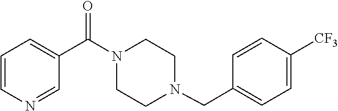 [SMILES: O=C(c1cccnc1)N1CCN(Cc2ccc(C(F)(F)F)cc2)CC1] | pyridin-3-yl(4-(4- (trifluoromethyl)benzyl)piperazin-1- yl)methanone |
| AA-582 | 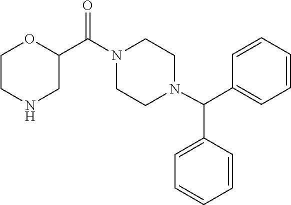 [SMILES: O=C(C1CNCCO1)N1CCN(C(c2ccccc2)c2ccccc2)CC1] | (4-benzhydrylpiperazin-1- yl)(morpholin-2-yl)methanone |
| AA-584 | 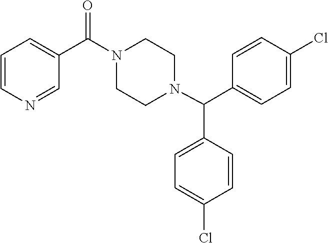 [SMILES: O=C(c1cccnc1)N1CCN(C(c2ccc(Cl)cc2)c2ccc(Cl)cc2)CC1] | (4-(bis(4- chlorophenyl)methyl)piperazin-1- yl)(pyridin-3-yl)methanone |
| AA-588 | 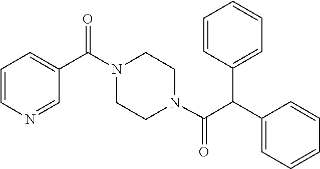 [SMILES: O=C(c1cccnc1)N1CCN(C(=O)C(c2ccccc2)c2ccccc2)CC1] | 1-(4-nicotinoylpiperazin-1-yl)-2,2- diphenylethan-1-one |
| AA-589 | 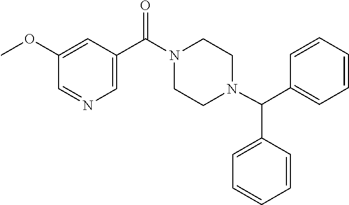 [SMILES: COc1cncc(C(=O)N2CCN(C(c3ccccc3)c3ccccc3)CC2)c1] | (4-benzhydrylpiperazin-1-yl)(5- methoxypyridin-3-yl)methanone |
| AA-590 | 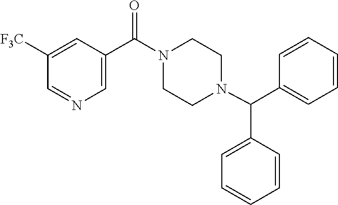 [SMILES: O=C(c1cncc(C(F)(F)F)c1)N1CCN(C(c2ccccc2)c2ccccc2)CC1] | (4-benzhydrylpiperazin-1-yl)(5- (trifluoromethyl)pyridin-3-yl)methanone |
| AA-591 | 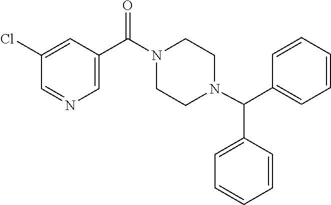 [SMILES: O=C(c1cncc(Cl)c1)N1CCN(C(c2ccccc2)c2ccccc2)CC1] | (4-benzhydrylpiperazin-1-yl)(5- chloropyridin-3-yl)methanone |
| AA-592 | 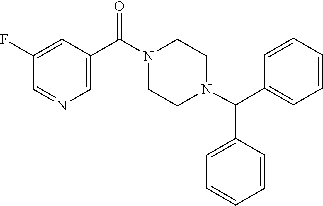 [SMILES: O=C(c1cncc(F)c1)N1CCN(C(c2ccccc2)c2ccccc2)CC1] | (4-benzhydrylpiperazin-1-yl)(5- fluoropyridin-3-yl)methanone |
| AA-596 | 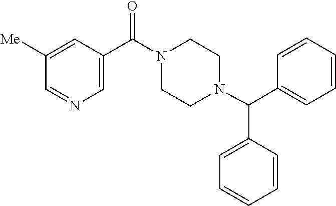 [SMILES: Cc1cncc(C(=O)N2CCN(C(c3ccccc3)c3ccccc3)CC2)c1] | (4-benzhydrylpiperazin-1-yl)(5- methylpyridin-3-yl)methanone |
| AA-597 | 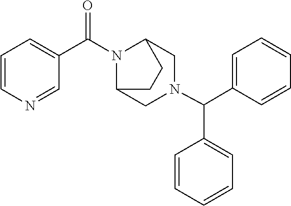 [SMILES: O=C(c1cccnc1)N1C2CCC1CN(C(c1ccccc1)c1ccccc1)C2] | (3-benzhydryl-3,8- diazabicyclo[3.2.1]octan-8-yl)(pyridin- 3-yl)methanone |
| AA-598 | 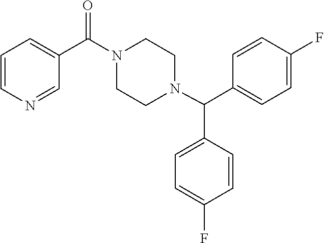 [SMILES: O=C(c1cccnc1)N1CCN(C(c2ccc(F)cc2)c2ccc(F)cc2)CC1] | (4-(bis(4- fluorophenyl)methyl)piperazin-1- yl)(yridine-3-yl)methanone |
| AA-599 | 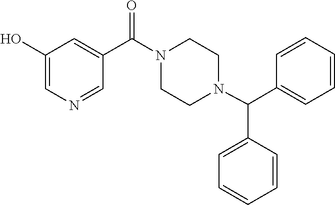 [SMILES: O=C(c1cncc(O)c1)N1CCN(C(c2ccccc2)c2ccccc2)CC1] | (4-benzhydrylpiperazin-1-yl)(5- hydroxypyridin-3-yl)methanone |
| AA-603 | 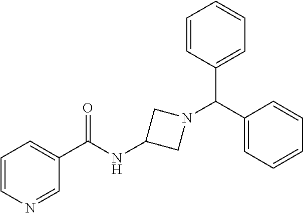 [SMILES: O=C(NC1CN(C(c2ccccc2)c2ccccc2)C1)c1cccnc1] | N-(1-benzhydrylazetidin-3- yl)nicotinamide |
| AA-604 | 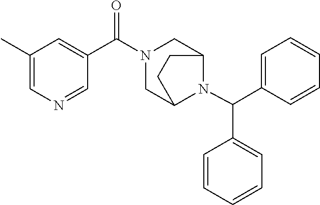 [SMILES: Cc1cncc(C(=O)N2CC3CCC(C2)N3C(c2ccccc2)c2ccccc2)c1] | (8-benzhydryl-3,8- diazabicyclo[3.2.1]octan-3-yl)(5- methylpyridin-3-yl)methanone |
| AA-605 | 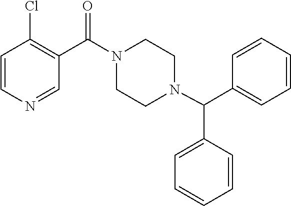 [SMILES: O=C(c1cnccc1Cl)N1CCN(C(c2ccccc2)c2ccccc2)CC1] | (4-benzhydrylpiperazin-1-yl)(4- chloropyridin-3-yl)methanone |
| AA-606 | 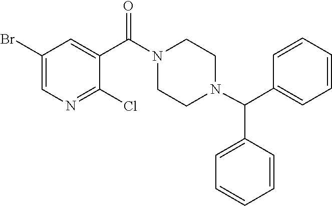 [SMILES: O=C(c1cc(Br)cnc1Cl)N1CCN(C(c2ccccc2)c2ccccc2)CC1] | (4-benzhydrylpiperazin-1-yl)(5-bromo- 2-chloropyridin-3-yl)methanone |
| AA-608 | 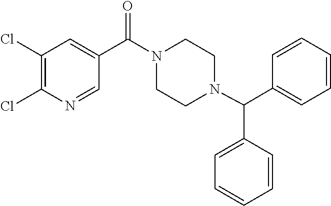 [SMILES: O=C(c1cnc(Cl)c(Cl)c1)N1CCN(C(c2ccccc2)c2ccccc2)CC1] | (4-benzhydrylpiperazin-1-yl)(5,6- dichloropyridin-3-yl)methanone |
| AA-607 | 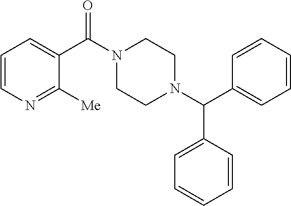 [SMILES: Cc1ncccc1C(=O)N1CCN(C(c2ccccc2)c2ccccc2)CC1] | (4-benzhydrylpiperazin-1-yl)(2- methylpyridin-3-yl)methanone |
| AA-611 | 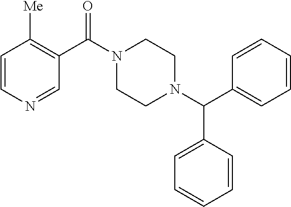 [SMILES: Cc1ccncc1C(=O)N1CCN(C(c2ccccc2)c2ccccc2)CC1] | (4-benzhydrylpiperazin-1-yl)(4- methylpyridin-3-yl)methanone |
| AA-612 | 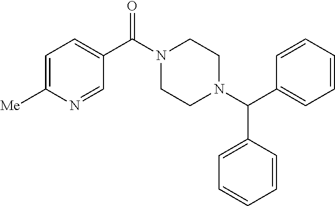 [SMILES: Cc1ccc(C(=O)N2CCN(C(c3ccccc3)c3ccccc3)CC2)cn1] | (4-benzhydrylpiperazin-1-yl)(6- methylpyridin-3-yl)methanone |
| AA-613 | 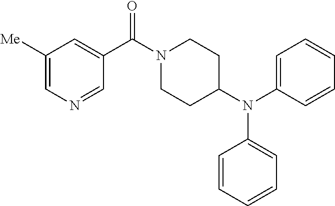 [SMILES: Cc1cncc(C(=O)N2CCC(N(c3ccccc3)c3ccccc3)CC2)c1] | (4-(diphenylamino)piperidin-1-yl)(5- methylpyridin-3-yl)methanone |
| AA-614 | 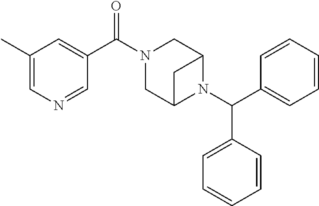 [SMILES: Cc1cncc(C(=O)N2CC3CC(C2)N3C(c2ccccc2)c2ccccc2)c1] | (6-benzhydryl-3,6- diazabicyclo[3.1.1]heptan-3-yl)(5- methylpyridin-3-yl)methanone |
| AA-620 | 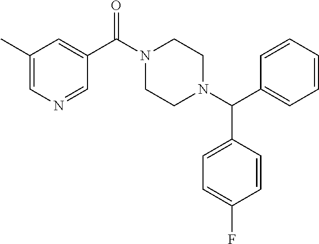 [SMILES: Cc1cncc(C(=O)N2CCN(C(c3ccccc3)c3ccc(F)cc3)CC2)c1] | (4-((4- fluorophenyl)(phenyl)methyl)piperazin- 1-yl)(5-methylpyridin-3-yl)methanone |
| AA-624 | 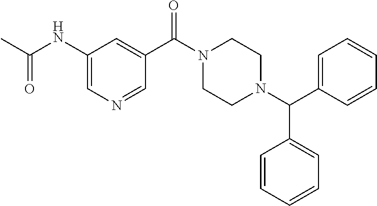 [SMILES: CC(=O)Nc1cncc(C(=O)N2CCN(C(c3ccccc3)c3ccccc3)CC2)c1] | N-(5-(4-benzhydrylpiperazine-1- carbonyl)pyridin-3-yl)acetamide |
| AA-625 | 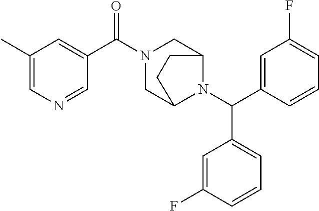 [SMILES: Cc1cncc(C(=O)N2CC3CCC(C2)N3C(c2cccc(F)c2)c2cccc(F)c2)c1] | (8-(bis(3-fluorophenyl)methyl)-3,8- diazabicyclo[3.2.1]octan-3-yl)(5- methylpyridin-3-yl)methanone |
| AA-627 | 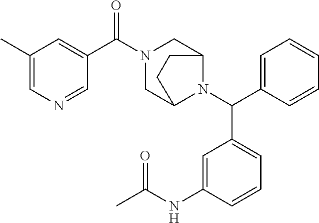 [SMILES: CC(=O)Nc1cccc(C(c2ccccc2)N2C3CCC2CN(C(=O)c2cncc(C)c2)C3)c1] | N-(3-((3-(5-methylnicotinoyl)-3,8- diazabicyclo[3.2.1]octan-8- yl)(phenyl)methyl)phenyl)acetamide |
| AA-630 | 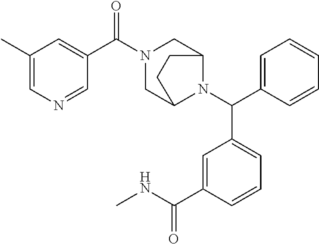 [SMILES: CNC(=O)c1cccc(C(c2ccccc2)N2C3CCC2CN(C(=O)c2cncc(C)c2)C3)c1] | N-methyl-3-((3-(5-methylnicotinoyl)- 3,8-diazabicyclo[3.2.1]octan-8- yl)(phenyl)methyl)benzamide |
| AA-633 | 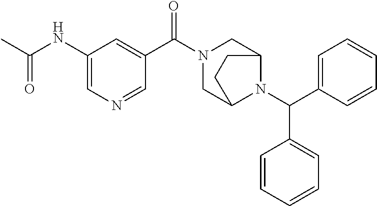 [SMILES: CC(=O)Nc1cncc(C(=O)N2CC3CCC(C2)N3C(c2ccccc2)c2ccccc2)c1] | N-(5-(8-benzhydryl-3,8- diazabicyclo[3.2.1]octane-3- carbonyl)pyridin-3-yl)acetamide |
| AA-637 | 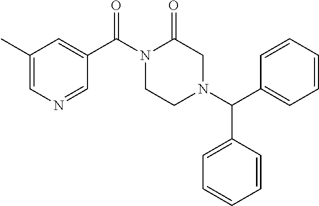 [SMILES: Cc1cncc(C(=O)N2CCN(C(c3ccccc3)c3ccccc3)CC2=O)c1] | 4-benzhydryl-1-(5- methylnicotinoyl)piperazin-2-one |
| AA-638 | 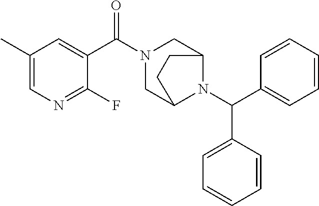 [SMILES: Cc1cnc(F)c(C(=O)N2CC3CCC(C2)N3C(c2ccccc2)c2ccccc2)c1] | (8-benzhydryl-3,8- diazabicyclo[3.2.1]octan-3-yl)(2-fluoro- 5-methylpyridin-3-yl)methanone |
| AA-642 | 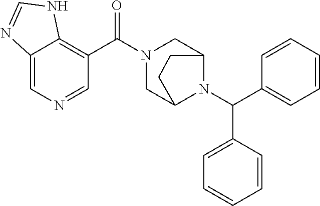 [SMILES: O=C(c1cncc2nc[nH]c12)N1CC2CCC(C1)N2C(c1ccccc1)c1ccccc1] | (8-benzhydryl-3,8- diazabicyclo[3.2.1]octan-3-yl)(1H- imidazo[4,5-c]pyridin-7-yl)methanone |
| AA-643 | 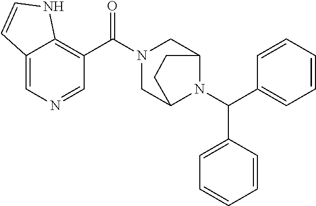 [SMILES: O=C(c1cncc2cc[nH]c12)N1CC2CCC(C1)N2C(c1ccccc1)c1ccccc1] | (8-benzhydryl-3,8- diazabicyclo[3.2.1]octan-3-yl)(1H- pyrrolo[3,2-c]pyridin-7-yl)methanone |
| AA-645 | 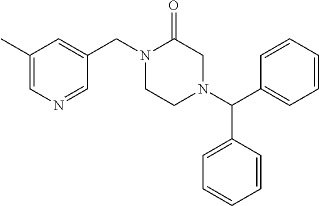 [SMILES: Cc1cncc(CN2CCN(C(c3ccccc3)c3ccccc3)CC2=O)c1] | 4-benzhydryl-1-((5-methylpyridin-3- yl)methyl)piperazin-2-one |
| AA-646 | 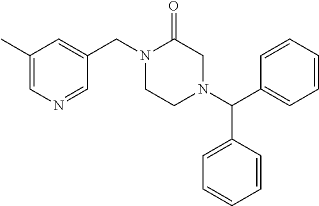 [SMILES: Cc1cncc(CN2CCN(C(c3ccccc3)c3ccccc3)CC2=O)c1] | 4-benzhydryl-1-((5-methylpyridin-3- yl)methyl)piperazin-2-one |
| MKP-1157 | 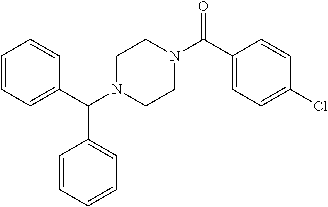 [SMILES: O=C(c1ccc(Cl)cc1)N1CCN(C(c2ccccc2)c2ccccc2)CC1] | 1-(4-chlorobenzoyl)-4- (diphenylmethyl)piperazine |
| MKP-1158 | 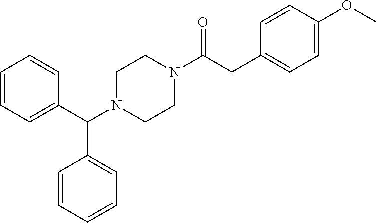 [SMILES: COc1ccc(CC(=O)N2CCN(C(c3ccccc3)c3ccccc3)CC2)cc1] | 1-[4-(diphenylmethyl)piperazin-1-yl]-2- (4-methoxyphenyl)ethan-1-one |
| MKP-1161 | 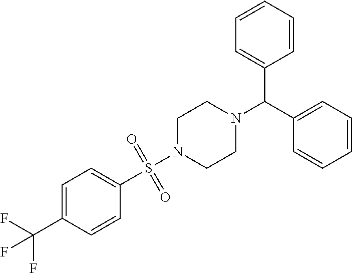 [SMILES: O=S(=O)(c1ccc(C(F)(F)F)cc1)N1CCN(C(c2ccccc2)c2ccccc2)CC1] | 1-(diphenylmethyl)-4-[4- (trifluoromethyl)benzenesulfonyl] piperazine |
| MKP-1162 | 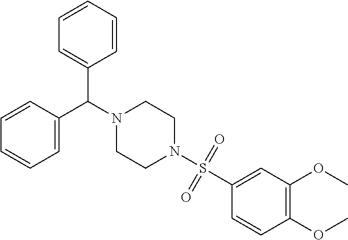 [SMILES: COc1ccc(S(=O)(=O)N2CCN(C(c3ccccc3)c3ccccc3)CC2)cc1OC] | 1-(3,4-dimethoxybenzenesulfonyl)-4- (diphenylmethyl)piperazine |
| MKP-1151 | 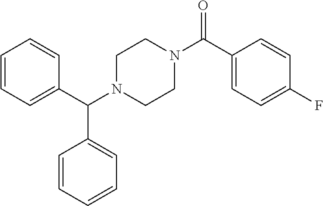 [SMILES: O=C(c1ccc(F)cc1)N1CCN(C(c2ccccc2)c2ccccc2)CC1] | 1-(diphenylmethyl)-4-(4- fluorobenzoyl)piperazine |
| MKP-1152 | 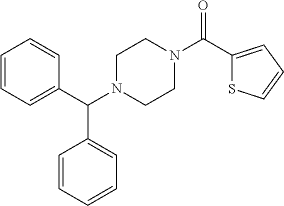 [SMILES: O=C(c1cccs1)N1CCN(C(c2ccccc2)c2ccccc2)CC1] | 1-(diphenylmethyl)-4-(thiophene-2- carbonyl)piperazine |
| MKP-1167 | 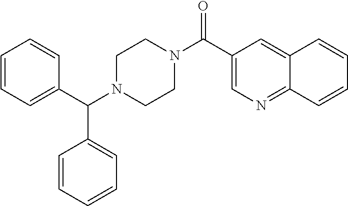 [SMILES: O=C(c1cnc2ccccc2c1)N1CCN(C(c2ccccc2)c2ccccc2)CC1] | 3-[4-(diphenylmethyl)piperazine-1- carbonyl]quinoline |
| MKP-1168 | 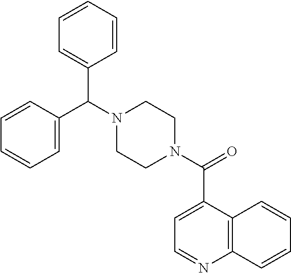 [SMILES: O=C(c1ccnc2ccccc12)N1CCN(C(c2ccccc2)c2ccccc2)CC1] | 4-[4-(diphenylmethyl)piperazine-1- carbonyl]quinoline |
| MKP-1176 | 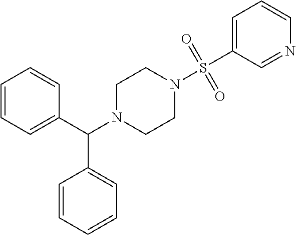 [SMILES: O=S(=O)(c1cccnc1)N1CCN(C(c2ccccc2)c2ccccc2)CC1] | 1-(diphenylmethyl)-4-(pyridine-3- sulfonyl)piperazine |
| MKP-1183 | 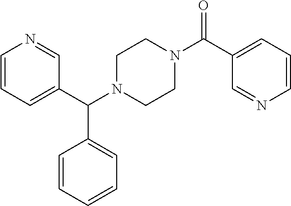 [SMILES: O=C(c1cccnc1)N1CCN(C(c2ccccc2)c2cccnc2)CC1] | 1-[phenyl(pyridin-3-yl)methyl]-4- (pyridine-3-carbonyl)piperazine |
| MKP-1188 | 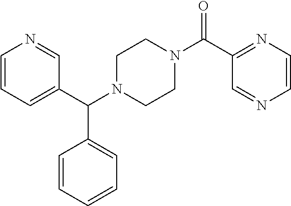 [SMILES: O=C(c1cnccn1)N1CCN(C(c2ccccc2)c2cccnc2)CC1] | 2-{4-[phenyl(pyridin-3- yl)methyl]piperazine-1- carbonyl}pyrazine |
| MKP-1189 | 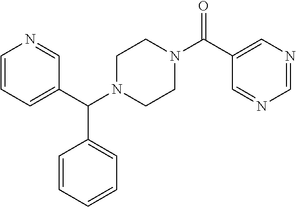 [SMILES: O=C(c1cncnc1)N1CCN(C(c2ccccc2)c2cccnc2)CC1] | 5-{4-[phenyl(pyridin-3- yl)methyl]piperazine-1- carbonyl}pyrimidine |
| MKP-1192 | 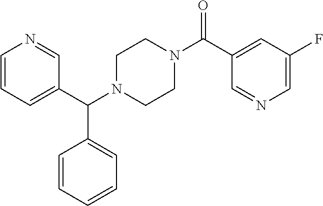 [SMILES: O=C(c1cncc(F)c1)N1CCN(C(c2ccccc2)c2cccnc2)CC1] | 1-(5-fluoropyridine-3-carbonyl)-4- [phenyl(pyridin-3-yl)methyl]piperazine |
| MKP-1193 | 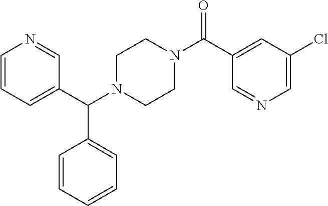 [SMILES: O=C(c1cncc(Cl)c1)N1CCN(C(c2ccccc2)c2cccnc2)CC1] | 1-(5-chloropyridine-3-carbonyl)-4- [phenyl(pyridin-3-yl)methyl]piperazine |
| MKP-1194 | 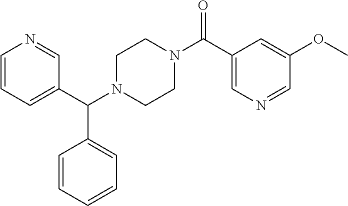 [SMILES: COc1cncc(C(=O)N2CCN(C(c3ccccc3)c3cccnc3)CC2)c1] | 1-(5-methoxypyridine-3-carbonyl)-4- [phenyl(pyridin-3-yl)methyl]piperazine |
| MKP-1195 | 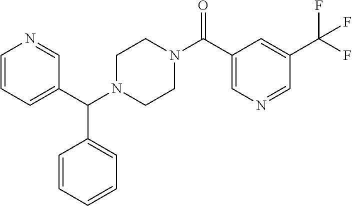 [SMILES: O=C(c1cncc(C(F)(F)F)c1)N1CCN(C(c2ccccc2)c2cccnc2)CC1] | 1-[phenyl(pyridin-3-yl)methyl]-4-[5- (trifluoromethyl)pyridine-3- carbonyl]piperazine |
| MKP-1201 | 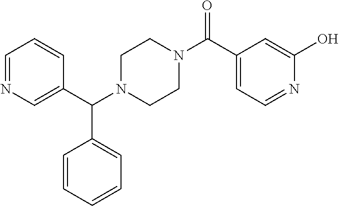 [SMILES: O=C(c1ccnc(O)c1)N1CCN(C(c2ccccc2)c2cccnc2)CC1] | 4-{4-[phenyl(pyridin-3- yl)methyl]piperazine-1- carbonyl}pyridin-2-ol |
| MKP-1204 | 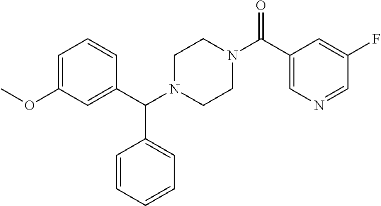 [SMILES: COc1cccc(C(c2ccccc2)N2CCN(C(=O)c3cncc(F)c3)CC2)c1] | 1-(5-fluoropyridine-3-carbonyl)-4-[(3- methoxyphenyl)(phenyl)methyl] piperazine |
| MKP-1215 | 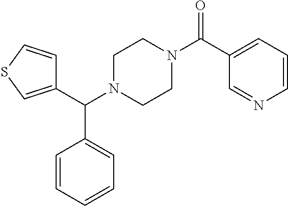 [SMILES: O=C(c1cccnc1)N1CCN(C(c2ccccc2)c2ccsc2)CC1] | 1-[phenyl(thiophen-3-yl)methyl]-4- (pyridine-3-carbonyl)piperazine |
| MKP-1216 | 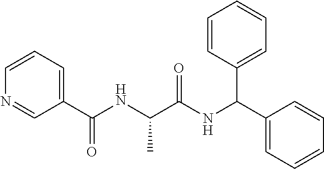 [SMILES: C[C@H](NC(=O)c1cccnc1)C(=O)NC(c1ccccc1)c1ccccc1] | (2S)-N-(diphenylmethyl)-2-[(pyridin-3- yl)formamido]propanamide |
| MKP-1200 | 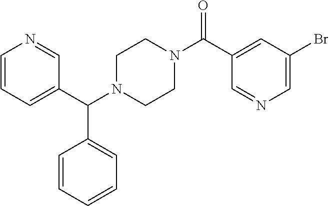 [SMILES: O=C(c1cncc(Br)c1)N1CCN(C(c2ccccc2)c2cccnc2)CC1] | 1-(5-bromopyridine-3-carbonyl)-4- [phenyl(pyridin-3-yl)methyl]piperazine |
| MKP-1203 | 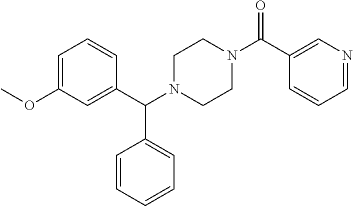 [SMILES: COc1cccc(C(c2ccccc2)N2CCN(C(=O)c3cccnc3)CC2)c1] | 1-[(3-methoxyphenyl)(phenyl)methyl]- 4-(pyridine-3-carbonyl)piperazine |
| MKP-1219 | 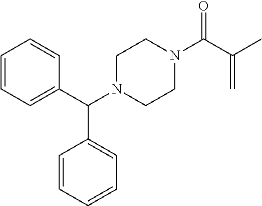 [SMILES: C=C(C)C(=O)N1CCN(C(c2ccccc2)c2ccccc2)CC1] | 1-[4-(diphenylmethyl)piperazin-1-yl]-2- methylprop-2-en-1-one |
| MKP-1205 | 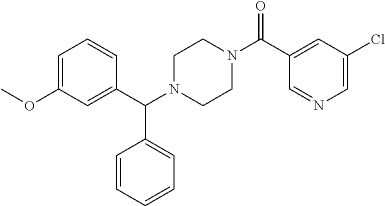 [SMILES: COc1cccc(C(c2ccccc2)N2CCN(C(=O)c3cncc(Cl)c3)CC2)c1] | 1-(5-chloropyridine-3-carbonyl)-4-[(3- methoxyphenyl)(phenyl)methyl] piperazine |
| MKP-1217 | 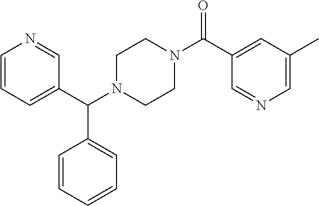 [SMILES: Cc1cncc(C(=O)N2CCN(C(c3ccccc3)c3cccnc3)CC2)c1] | 1-(5-methylpyridine-3-carbonyl)-4- [phenyl(pyridin-3-yl)methyl]piperazine |
| MKP-1220 | 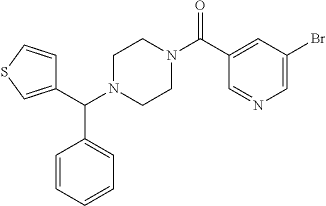 [SMILES: O=C(c1cncc(Br)c1)N1CCN(C(c2ccccc2)c2ccsc2)CC1] | 1-(5-bromopyridine-3-carbonyl)-4- [phenyl(thiophen-3- yl)methyl]piperazine |
| MKP-1221 | 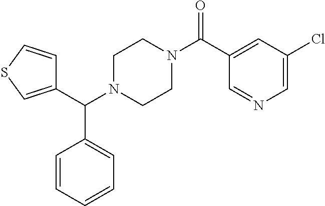 [SMILES: O=C(c1cncc(Cl)c1)N1CCN(C(c2ccccc2)c2ccsc2)CC1] | 1-(5-chloropyridine-3-carbonyl)-4- [phenyl(thiophen-3- yl)methyl]piperazine |
| MKP-1222 | 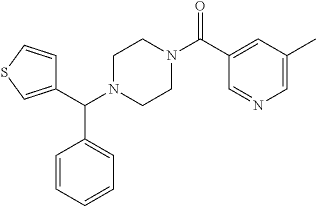 [SMILES: Cc1cncc(C(=O)N2CCN(C(c3ccccc3)c3ccsc3)CC2)c1] | 1-(5-methylpyridine-3-carbonyl)-4- [phenyl(thiophen-3- yl)methyl]piperazine |
| MKP-1225 | 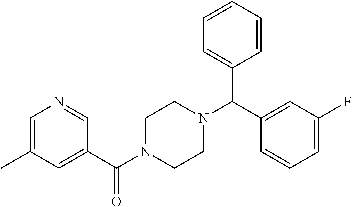 [SMILES: Cc1cncc(C(=O)N2CCN(C(c3ccccc3)c3cccc(F)c3)CC2)c1] | 1-[(3-fluorophenyl)(phenyl)methyl]-4- (5-methylpyridine-3- carbonyl)piperazine |
| MKP-1229 | 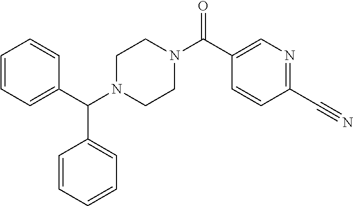 [SMILES: N#Cc1ccc(C(=O)N2CCN(C(c3ccccc3)c3ccccc3)CC2)cn1] | 5-[4-(diphenylmethyl)piperazine-1- carbonyl]pyridine-2-carbonitrile |
| MKP-1230 | 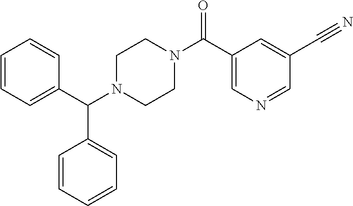 [SMILES: N#Cc1cncc(C(=O)N2CCN(C(c3ccccc3)c3ccccc3)CC2)c1] | 5-[4-(diphenylmethyl)piperazine-1- carbonyl]pyridine-3-carbonitrile |
| MKP-1268 | 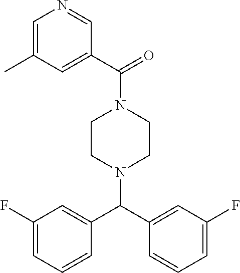 [SMILES: Cc1cncc(C(=O)N2CCN(C(c3cccc(F)c3)c3cccc(F)c3)CC2)c1] | 1-[bis(3-fluorophenyl)methyl]-4-(5- methylpyridine-3-carbonyl)piperazine |
| MKP-1272 | 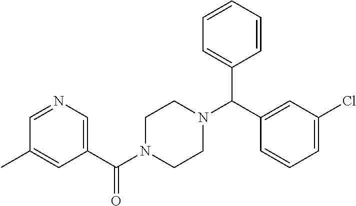 [SMILES: Cc1cncc(C(=O)N2CCN(C(c3ccccc3)c3cccc(Cl)c3)CC2)c1] | 1-[(3-chlorophenyl)(phenyl)methyl]-4- (5-methylpyridine-3- carbonyl)piperazine |
| MKP-1227 | 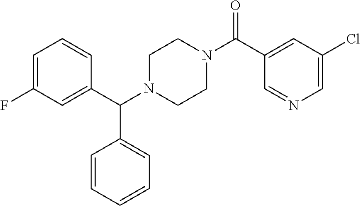 [SMILES: O=C(c1cncc(Cl)c1)N1CCN(C(c2ccccc2)c2cccc(F)c2)CC1] | 1-(5-chloropyridine-3-carbonyl)-4-[(3- fluorophenyl)(phenyl)methyl]piperazine |
| MKP-1276 | 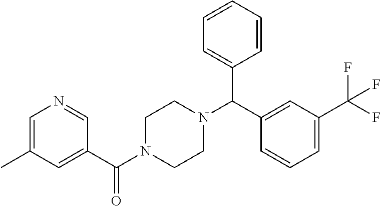 [SMILES: Cc1cncc(C(=O)N2CCN(C(c3ccccc3)c3cccc(C(F)(F)F)c3)CC2)c1] | 1-(5-methylpyridine-3-carbonyl)-4- {phenyl[3- (trifluoromethyl)phenyl]methyl} piperazine |
| MKP-1280 | 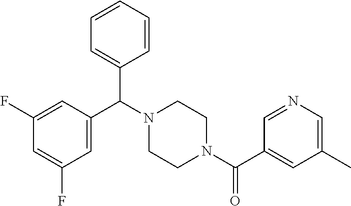 [SMILES: Cc1cncc(C(=O)N2CCN(C(c3ccccc3)c3cc(F)cc(F)c3)CC2)c1] | 1-[(3,5-difluorophenyl)(phenyl)methyl]- 4-(5-methylpyridine-3- carbonyl)piperazine |
| MKP-1277 | 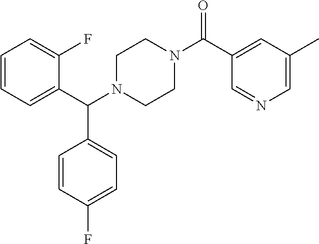 [SMILES: Cc1cncc(C(=O)N2CCN(C(c3ccc(F)cc3)c3ccccc3F)CC2)c1] | 1-[(2-fluorophenyl)(4- fluorophenyl)methyl]-4-(5- methylpyridine-3-carbonyl)piperazine |
| MKP-1290 | 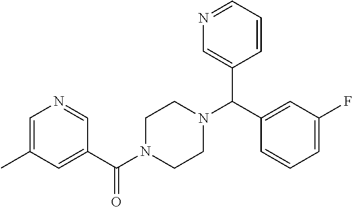 [SMILES: Cc1cncc(C(=O)N2CCN(C(c3cccnc3)c3cccc(F)c3)CC2)c1] | 1-[(3-fluorophenyl)(pyridin-3- yl)methyl]-4-(5-methylpyridine-3- carbonyl)piperazine |
| MKP-1304 | 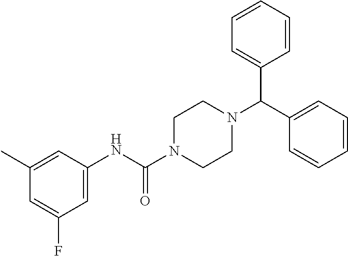 [SMILES: Cc1cc(F)cc(NC(=O)N2CCN(C(c3ccccc3)c3ccccc3)CC2)c1] | 4-(diphenylmethyl)-N-(3-fluoro-5- methylphenyl)piperazine-1-carboxamide |
| MKP-1307 | 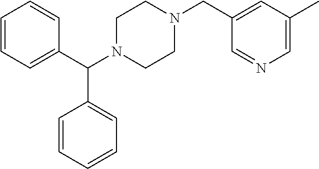 [SMILES: Cc1cncc(CN2CCN(C(c3ccccc3)c3ccccc3)CC2)c1] | 1-(diphenylmethyl)-4-[(5- methylpyridin-3-yl)methyl]piperazine |
| MKP-1287 | 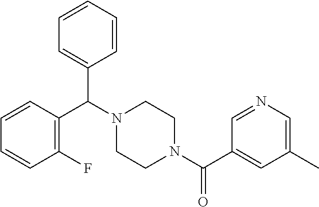 [SMILES: Cc1cncc(C(=O)N2CCN(C(c3ccccc3)c3ccccc3F)CC2)c1] | 1-[(2-fluorophenyl)(phenyl)methyl]-4- (5-methylpyridine-3- carbonyl)piperazine |
| MKP-1306 | 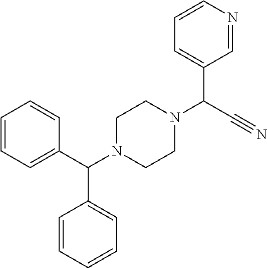 [SMILES: N#CC(c1cccnc1)N1CCN(C(c2ccccc2)c2ccccc2)CC1] | 2-[4-(diphenylmethyl)piperazin-1-yl]-2- (pyridin-3-yl)acetonitrile |
| MKP-1309 | 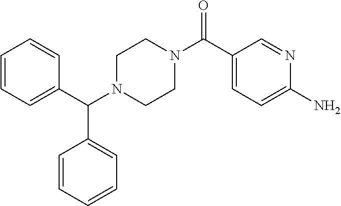 [SMILES: Nc1ccc(C(=O)N2CCN(C(c3ccccc3)c3ccccc3)CC2)cn1] | 5-[4-(diphenylmethyl)piperazine-1- carbonyl]pyridin-2-amine |
| MKP-1318 | 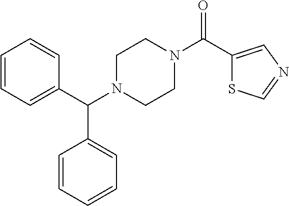 [SMILES: O=C(c1cncs1)N1CCN(C(c2ccccc2)c2ccccc2)CC1] | 1-(diphenylmethyl)-4-(1,3-thiazole-5- carbonyl)piperazine |
| MKP-1319 | 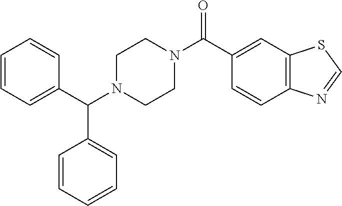 [SMILES: O=C(c1ccc2ncsc2c1)N1CCN(C(c2ccccc2)c2ccccc2)CC1] | 6-[4-(diphenylmethyl)piperazine-1- carbonyl]-1,3-benzothiazole |
| MKP-1322 | 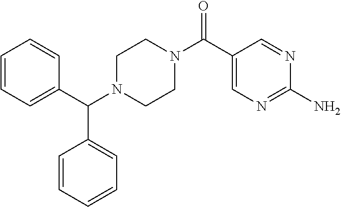 [SMILES: Nc1ncc(C(=O)N2CCN(C(c3ccccc3)c3ccccc3)CC2)cn1] | 5-[4-(diphenylmethyl)piperazine-1- carbonyl]pyrimidin-2-amine |
| MKP-1323 | 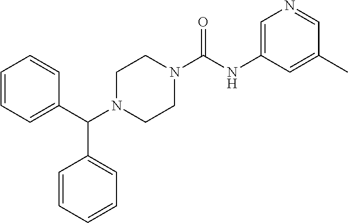 [SMILES: Cc1cncc(NC(=O)N2CCN(C(c3ccccc3)c3ccccc3)CC2)c1] | 4-benzhydryl-N-(5-methylpyridin-3- yl)piperazine-1-carboxamide |
| MKP-1324 | 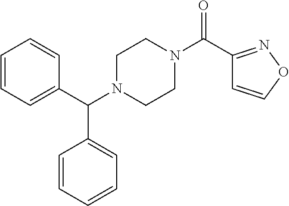 [SMILES: O=C(c1ccon1)N1CCN(C(c2ccccc2)c2ccccc2)CC1] | 1-(diphenylmethyl)-4-(1,2-oxazole-3- carbonyl)piperazine |
| NZ-806 | 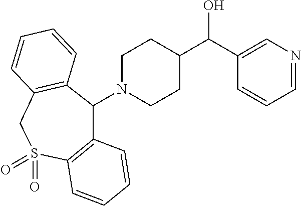 [SMILES: O=S1(=O)Cc2ccccc2C(N2CCC(C(O)c3cccnc3)CC2)c2ccccc21] | [1-(5,5-dioxo-6,11- dihydrobenzo[c][1]benzothiepin-11-yl)- 4-piperidyl]-(3-pyridyl)methanol |
| NZ-807 | 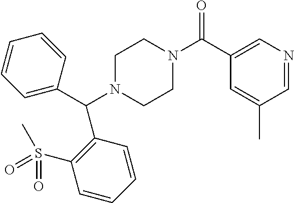 [SMILES: Cc1cncc(C(=O)N2CCN(C(c3ccccc3)c3ccccc3S(C)(=O)=O)CC2)c1] | (5-methyl-3-pyridyl)-[4-[(2- methylsulfonylphenyl)-phenyl- methyl]piperazin-1-yl]methanone |
| NZ-808 | 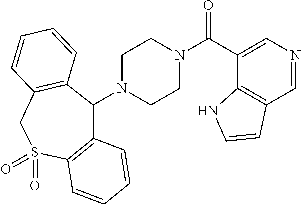 [SMILES: O=C(c1cncc2cc[nH]c12)N1CCN(C2c3ccccc3CS(=O)(=O)c3ccccc32)CC1] | [4-(5,5-dioxo-6,11- dihydrobenzo[c][1]benzothiepin-11- yl)piperazin-1-yl]-(1H-pyrrolo[3,2- c]pyridin-7-yl)methanone |
| NZ-809 | 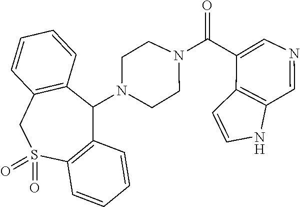 [SMILES: O=C(c1cncc2[nH]ccc12)N1CCN(C2c3ccccc3CS(=O)(=O)c3ccccc32)CC1] | [4-(5,5-dioxo-6,11- dihydrobenzo[c][1]benzothiepin-11- yl)piperazin-1-yl]-(1H-pyrrolo[2,3- c]pyridin-4-yl)methanone |
| NZ-810 | 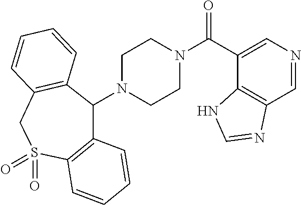 [SMILES: O=C(c1cncc2nc[nH]c12)N1CCN(C2c3ccccc3CS(=O)(=O)c3ccccc32)CC1] | [4-(5,5-dioxo-6,11- dihydrobenzo[c][1]benzothiepin-11- yl)piperazin-1-yl]-(1H-imidazo[4,5- c]pyridin-7-yl)methanone |
| NZ-811 | 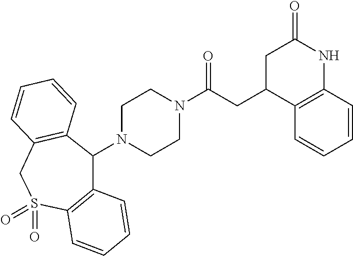 [SMILES: O=C1CC(CC(=O)N2CCN(C3c4ccccc4CS(=O)(=O)c4ccccc43)CC2)c2ccccc2N1] | 4-[2-[4-(5,5-dioxo-6,11- dihydrobenzo[c][1]benzothiepin-11- yl)piperazin-1-yl]-2-oxo-ethyl]-3,4- dihydro-1H-quinolin-2-one |
| NZ-812 | 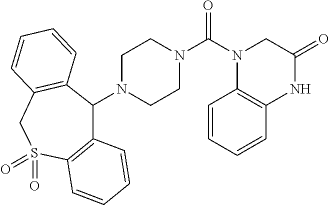 [SMILES: O=C1CN(C(=O)N2CCN(C3c4ccccc4CS(=O)(=O)c4ccccc43)CC2)c2ccccc2N1] | 4-[4-(5,5-dioxo-6,11- dihydrobenzo[c][1]benzothiepin-11- yl)piperazine-1-carbonyl]-1,3- dihydroquinoxalin-2-one |
| NZ-813 | 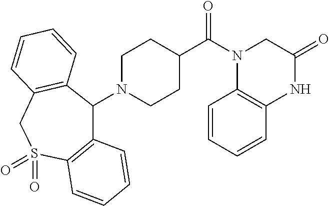 [SMILES: O=C1CN(C(=O)C2CCN(C3c4ccccc4CS(=O)(=O)c4ccccc43)CC2)c2ccccc2N1] | 4-[1-(5,5-dioxo-6,11- dihydrobenzo[c][1]benzothiepin-11- yl)piperidine-4-carbonyl]-1,3- dihydroquinoxalin-2-one |
| NZ-814 | 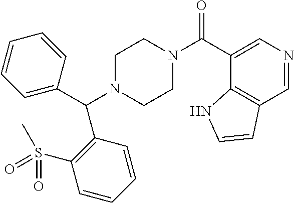 [SMILES: CS(=O)(=O)c1ccccc1C(c1ccccc1)N1CCN(C(=O)c2cncc3cc[nH]c23)CC1] | [4-[(2-methylsulfonylphenyl)-phenyl- methyl]piperazin-1-yl]-(1H-pyrrolo[3,2- c]pyridin-7-yl)methanone |
| NZ-815 | 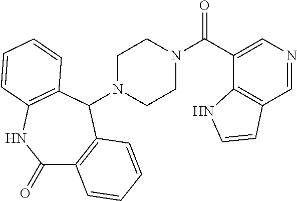 [SMILES: O=C1Nc2ccccc2C(N2CCN(C(=O)c3cncc4cc[nH]c34)CC2)c2ccccc21] | 11-[4-(1H-pyrrolo[3,2-c]pyridine-7- carbonyl)piperazin-1-yl]-5,11- dihydrobenzo[c][1]benzazepin-6-one |
| NZ-816 | 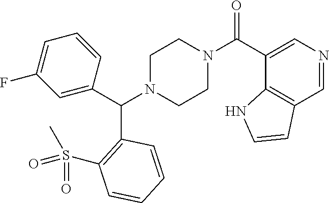 [SMILES: CS(=O)(=O)c1ccccc1C(c1cccc(F)c1)N1CCN(C(=O)c2cncc3cc[nH]c23)CC1] | [4-[(3-fluorophenyl)-(2- methylsulfonylphenyl)methyl]piperazin- 1-yl]-(1H-pyrrolo[3,2-c]pyridin-7- yl)methanone |
| NZ-817 | 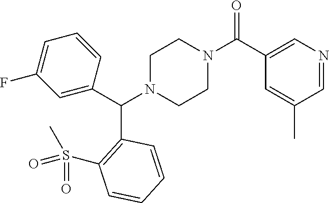 [SMILES: Cc1cncc(C(=O)N2CCN(C(c3cccc(F)c3)c3ccccc3S(C)(=O)=O)CC2)c1] | [4-[(3-fluorophenyl)-(2- methylsulfonylphenyl)methyl]piperazin- 1-yl]-(5-methyl-3-pyridyl)methanone |
| NZ-818 | 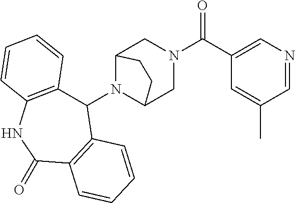 [SMILES: Cc1cncc(C(=O)N2CC3CCC(C2)N3C2c3ccccc3NC(=O)c3ccccc32)c1] | 11-[3-(5-methylpyridine-3-carbonyl)- 3,8-diazabicyclo[3.2.1]octan-8-yl]-5,11- dihydrobenzo[c][1]benzazepin-6-one |
| NZ-819 | 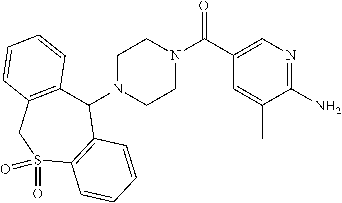 [SMILES: Cc1cc(C(=O)N2CCN(C3c4ccccc4CS(=O)(=O)c4ccccc43)CC2)cnc1N] | (6-amino-5-methyl-3-pyridyl)-[4-(5,5- dioxo-6,11- dihydrobenzo[c][1]benzothiepin-11- yl)piperazin-1-yl]methanone |
| NZ-820 | 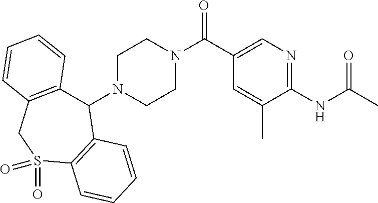 [SMILES: CC(=O)Nc1ncc(C(=O)N2CCN(C3c4ccccc4CS(=O)(=O)c4ccccc43)CC2)cc1C] | N-[5-[4-(5,5-dioxo-6,11- dihydrobenzo[c][1]benzothiepin-11- yl)piperazine-1-carbonyl]-3-methyl-2- pyridyl]acetamide |
| NZ-821 | 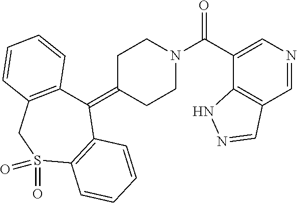 [SMILES: O=C(c1cncc2cn[nH]c12)N1CCC(=C2c3ccccc3CS(=O)(=O)c3ccccc32)CC1] | [4-(5,5-dioxo-6H- benzo[c][1]benzothiepin-11-ylidene)-1- piperidyl]-(1H-pyrazolo[4,3-c]pyridin- 7-yl)methanone |
| NZ-822 | 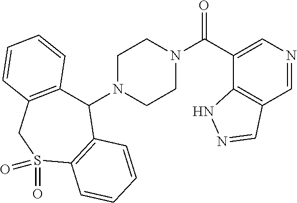 [SMILES: O=C(c1cncc2cn[nH]c12)N1CCN(C2c3ccccc3CS(=O)(=O)c3ccccc32)CC1] | [4-(5,5-dioxo-6,11- dihydrobenzo[c][1]benzothiepin-11- yl)piperazin-1-yl]-(1H-pyrazolo[4,3- c]pyridin-7-yl)methanone |
| NZ-823 | 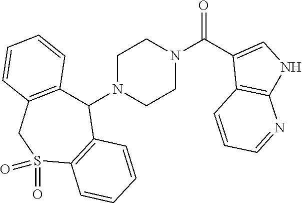 [SMILES: O=C(c1c[nH]c2ncccc12)N1CCN(C2c3ccccc3CS(=O)(=O)c3ccccc32)CC1] | [4-(5,5-dioxo-6,11- dihydrobenzo[c][1]benzothiepin-11- yl)piperazin-1-yl]-(1H-pyrrolo[2,3- b]pyridin-3-yl)methanone |
| AA-649 | 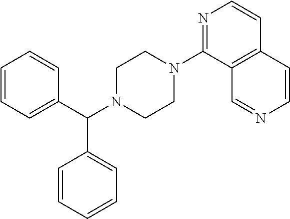 [SMILES: c1ccc(C(c2ccccc2)N2CCN(c3nccc4ccncc34)CC2)cc1] | 1-(4-benzhydrylpiperazin-1-yl)-2,7- naphthyridine |
| AA-652 | 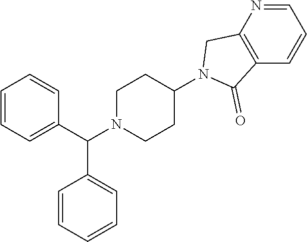 [SMILES: O=C1c2cccnc2CN1C1CCN(C(c2ccccc2)c2ccccc2)CC1] | 6-(1-benzhydryl-4-piperidyl)-7H- pyrrolo[3,4-b]pyridin-5-one |
| AA-654 | 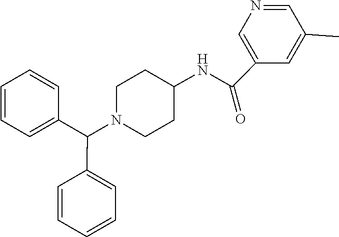 [SMILES: Cc1cncc(C(=O)NC2CCN(C(c3ccccc3)c3ccccc3)CC2)c1] | N-(1-benzhydryl-4-piperidyl)-5-methyl- pyridine-3-carboxamide |
| AA-655 | 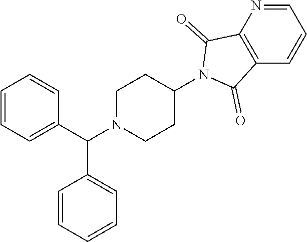 [SMILES: O=C1c2cccnc2C(=O)N1C1CCN(C(c2ccccc2)c2ccccc2)CC1] | 6-(1-benzhydryl-4- piperidyl)pyrrolo[3,4-b]pyridine-5,7- dione |
| AA-658 | 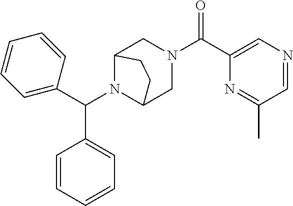 [SMILES: Cc1cncc(C(=O)N2CC3CCC(C2)N3C(c2ccccc2)c2ccccc2)n1] | (8-benzhydryl-3,8- diazabicyclo[3.2.1]octan-3-yl)-(6- methylpyrazin-2-yl)methanone |
| AA-660 | 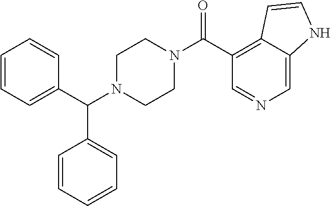 [SMILES: O=C(c1cncc2[nH]ccc12)N1CCN(C(c2ccccc2)c2ccccc2)CC1] | (4-benzhydrylpiperazin-1-yl)-(1H- pyrrolo[2,3-c]pyridin-4-yl)methanone |
| AA-661 | 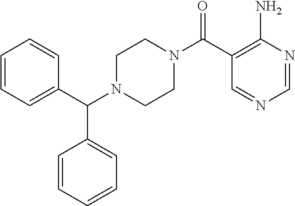 [SMILES: Nc1ncncc1C(=O)N1CCN(C(c2ccccc2)c2ccccc2)CC1] | (4-aminopyrimidin-5-yl)-(4- benzhydrylpiperazin-1-yl)methanone |
| AA-663 | 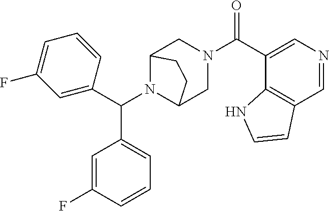 [SMILES: O=C(c1cncc2cc[nH]c12)N1CC2CCC(C1)N2C(c1cccc(F)c1)c1cccc(F)c1] | [8-[bis(3-fluorophenyl)methyl]-3,8- diazabicyclo[3.2.1]octan-3-yl]-(1H- pyrrolo[3,2-c]pyridin-7-yl)methanone |
| AA-664 | 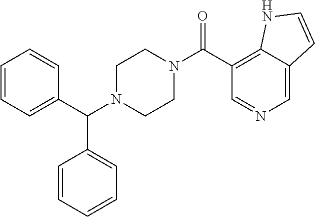 [SMILES: O=C(c1cncc2cc[nH]c12)N1CCN(C(c2ccccc2)c2ccccc2)CC1] | (4-benzhydrylpiperazin-1-yl)-(1H- pyrrolo[3,2-c]pyridin-7-yl)methanone |
| AA-665 | 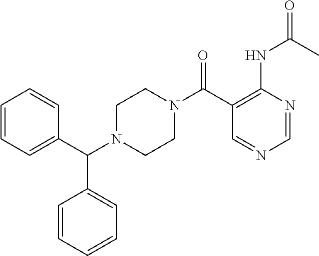 [SMILES: CC(=O)Nc1ncncc1C(=O)N1CCN(C(c2ccccc2)c2ccccc2)CC1] | N-[5-(4-benzhydrylpiperazine-1- carbonyl)pyrimidin-4-yl]acetamide |
| AA-666 | 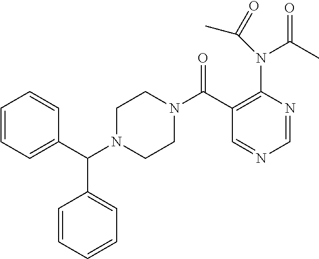 [SMILES: CC(=O)N(C(C)=O)c1ncncc1C(=O)N1CCN(C(c2ccccc2)c2ccccc2)CC1] | N-acetyl-N-[5-(4-benzhydrylpiperazine- 1-carbonyl)pyrimidin-4-yl]acetamide |
| AA-667 | 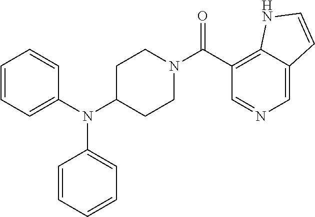 [SMILES: O=C(c1cncc2cc[nH]c12)N1CCC(N(c2ccccc2)c2ccccc2)CC1] | [4-(N-phenylanilino)-1-piperidyl]-(1H- pyrrolo[3,2-c]pyridin-7-yl)methanone |
| MKP-1331 | 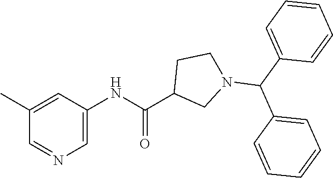 [SMILES: Cc1cncc(NC(=O)C2CCN(C(c3ccccc3)c3ccccc3)C2)c1] | 1-benzhydryl-N-(5-methylpyridin-3- yl)pyrrolidine-3-carboxamide |
| MKP-1332 | 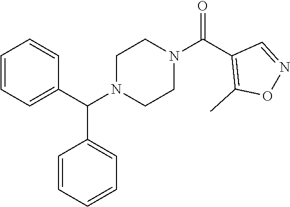 [SMILES: Cc1oncc1C(=O)N1CCN(C(c2ccccc2)c2ccccc2)CC1] | (4-benzhydrylpiperazin-1-yl)(5- methylisoxazol-4-yl)methanone |
| MKP-1335 | 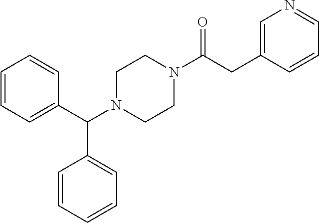 [SMILES: O=C(Cc1cccnc1)N1CCN(C(c2ccccc2)c2ccccc2)CC1] | 1-(4-benzhydrylpiperazin-1-yl)-2- (pyridin-3-yl)ethan-1-one |
| MKP-1338 | 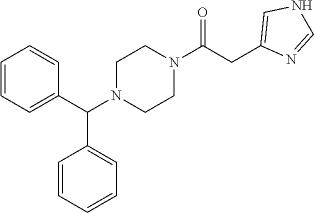 [SMILES: O=C(Cc1c[nH]cn1)N1CCN(C(c2ccccc2)c2ccccc2)CC1] | 1-(4-benzhydrylpiperazin-1-yl)-2-(1H- imidazol-4-yl)ethan-1-one |
| MKP-1344 | 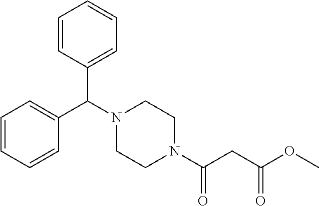 [SMILES: COC(=O)CC(=O)N1CCN(C(c2ccccc2)c2ccccc2)CC1] | methyl 3-(4-benzhydrylpiperazin-1-yl)- 3-oxopropanoate |
| MKP-1349 | 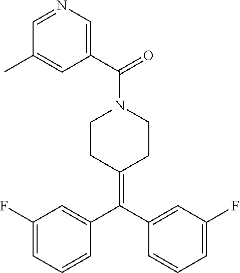 [SMILES: Cc1cncc(C(=O)N2CCC(=C(c3cccc(F)c3)c3cccc(F)c3)CC2)c1] | (4-(bis(3- fluorophenyl)methylene)piperidin-1- yl)(5-methylpyridin-3-yl)methanone |
| MKP-1355 | 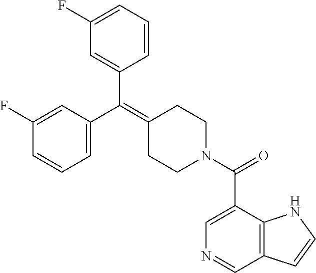 [SMILES: O=C(c1cncc2cc[nH]c12)N1CCC(=C(c2cccc(F)c2)c2cccc(F)c2)CC1] | (4-(bis(3- fluorophenyl)methylene)piperidin-1- yl)(1H-pyrrolo[3,2-c]pyridin-7- yl)methanone |
| MKP-1359 | 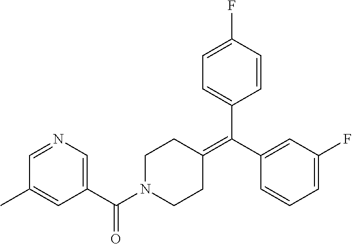 [SMILES: Cc1cncc(C(=O)N2CCC(=C(c3ccc(F)cc3)c3cccc(F)c3)CC2)c1] | (4-((3-fluorophenyl)(4- fluorophenyl)methylene)piperidin-1- yl)(5-methylpyridin-3-yl)methanone |
| MKP-1360 | 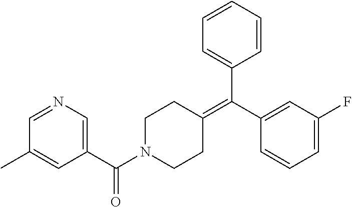 [SMILES: Cc1cncc(C(=O)N2CCC(=C(c3ccccc3)c3cccc(F)c3)CC2)c1] | (4-((3- fluorophenyl)(phenyl)methylene) piperidin-1-yl)(5-methylpyridin-3- yl)methanone |
| MKP-1371 | 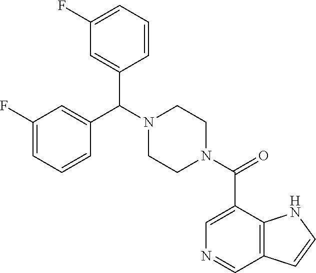 [SMILES: O=C(c1cncc2cc[nH]c12)N1CCN(C(c2cccc(F)c2)c2cccc(F)c2)CC1] | (4-(bis(3- fluorophenyl)methyl)piperazin-1- yl)(1H-pyrrolo[3,2-c]pyridin-7- yl)methanone |
| MKP-1381 | 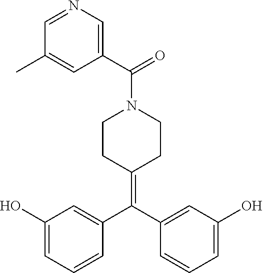 [SMILES: Cc1cncc(C(=O)N2CCC(=C(c3cccc(O)c3)c3cccc(O)c3)CC2)c1] | (4-(bis(3- hydroxyphenyl)methylene)piperidin-1- yl)(5-methylpyridin-3-yl)methanone |
| MKP-1392 | 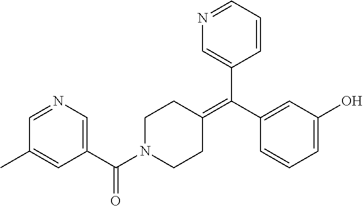 [SMILES: Cc1cncc(C(=O)N2CCC(=C(c3cccnc3)c3cccc(O)c3)CC2)c1] | (4-((3-hydroxyphenyl)(pyridin-3- yl)methylene)piperidin-1-yl)(5- methylpyridin-3-yl)methanone |
| MKP-1398 | 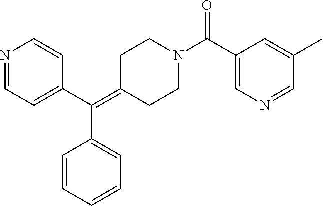 [SMILES: Cc1cncc(C(=O)N2CCC(=C(c3ccccc3)c3ccncc3)CC2)c1] | (5-methylpyridin-3-yl)(4- (phenyl(pyridin-4- yl)methylene)piperidin-1-yl)methanone |
| MKP-1400 | 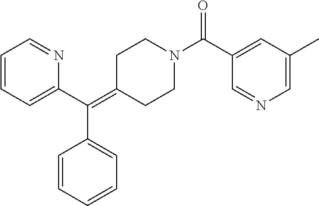 [SMILES: Cc1cncc(C(=O)N2CCC(=C(c3ccccc3)c3ccccn3)CC2)c1] | (5-methylpyridin-3-yl)(4- (phenyl(pyridin-2- yl)methylene)piperidin-1-yl)methanone |
| MKP-1404 | 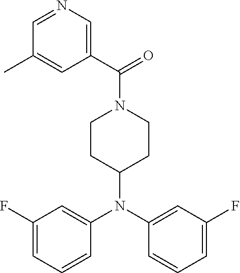 [SMILES: Cc1cncc(C(=O)N2CCC(N(c3cccc(F)c3)c3cccc(F)c3)CC2)c1] | (4-(bis(3-fluorophenyl)amino)piperidin- 1-yl)(5-methylpyridin-3-yl)methanone |
| MKP-1410 | 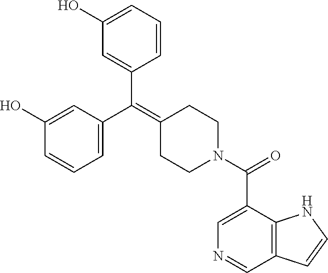 [SMILES: O=C(c1cncc2cc[nH]c12)N1CCC(=C(c2cccc(O)c2)c2cccc(O)c2)CC1] | (4-(bis(3- hydroxyphenyl)methylene)piperidin-1- yl)(1H-pyrrolo[3,2-c]pyridin-7- yl)methanone |
| MKP-1416 | 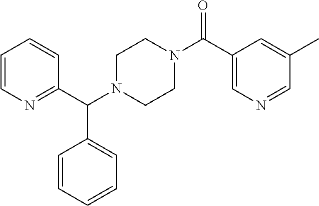 [SMILES: Cc1cncc(C(=O)N2CCN(C(c3ccccc3)c3ccccn3)CC2)c1] | (5-methylpyridin-3-yl)(4- (phenyl(pyridin-2-yl)methyl)piperazin- 1-yl)methanone |
| MKP-1421 | 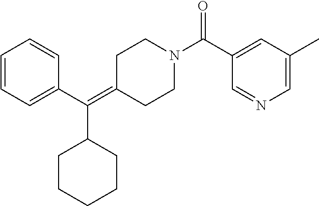 [SMILES: Cc1cncc(C(=O)N2CCC(=C(c3ccccc3)C3CCCCC3)CC2)c1] | (4- (cyclohexyl(phenyl)methylene)piperidin- 1-yl)(5-methylpyridin-3-yl)methanone |
| MKP-1422 | 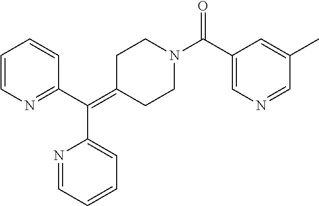 [SMILES: Cc1cncc(C(=O)N2CCC(=C(c3ccccn3)c3ccccn3)CC2)c1] | (4-(di(pyridin-2-yl)methylene)piperidin- 1-yl)(5-methylpyridin-3-yl)methanone |
| MKP-1440 | 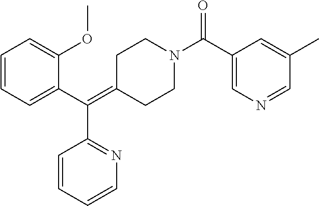 [SMILES: COc1ccccc1C(=C1CCN(C(=O)c2cncc(C)c2)CC1)c1ccccn1] | (4-((2-methoxyphenyl)(pyridin-2- yl)methylene)piperidin-1-yl)(5- methylpyridin-3-yl)methanone |
| MKP-1442 | 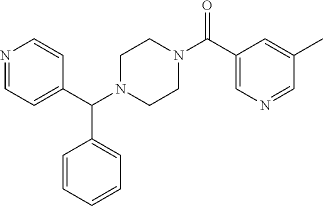 [SMILES: Cc1cncc(C(=O)N2CCN(C(c3ccccc3)c3ccncc3)CC2)c1] | (5-methylpyridin-3-yl)(4- (phenyl(pyridin-4-yl)methyl)piperazin- 1-yl)methanone |
| MKP-1445 | 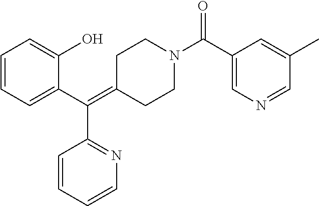 [SMILES: Cc1cncc(C(=O)N2CCC(=C(c3ccccn3)c3ccccc3O)CC2)c1] | (4-((2-hydroxyphenyl)(pyridin-2- yl)methylene)piperidin-1-yl)(5- methylpyridin-3-yl)methanone |
| MKP-1449 | 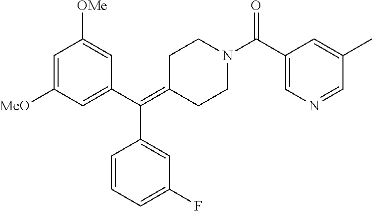 [SMILES: COc1cc(OC)cc(C(=C2CCN(C(=O)c3cncc(C)c3)CC2)c2cccc(F)c2)c1] | (4-((3,5-dimethoxyphenyl)(3- fluorophenyl)methylene)piperidin-1- yl)(5-methylpyridin-3-yl)methanone |
| MKP-1450 | 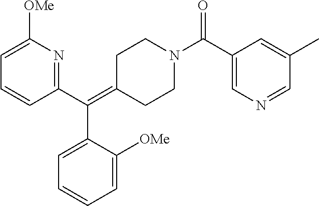 [SMILES: COc1cccc(C(=C2CCN(C(=O)c3cncc(C)c3)CC2)c2ccccc2OC)n1] | (4-((2-methoxyphenyl)(6- methoxypyridin-2- yl)methylene)piperidin-1-yl)(5- methylpyridin-3-yl)methanone |
| MKP-1451 | 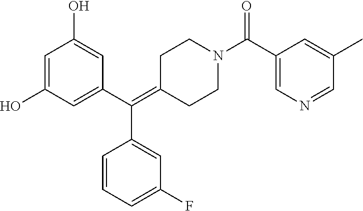 [SMILES: Cc1cncc(C(=O)N2CCC(=C(c3cc(O)cc(O)c3)c3cccc(F)c3)CC2)c1] | (4-((3,5-dihydroxyphenyl)(3- fluorophenyl)methylene)piperidin-1- yl)(5-methylpyridin-3-yl)methanone |
| PKJ-595 | 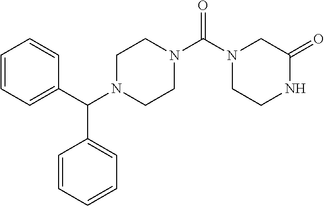 [SMILES: O=C1CN(C(=O)N2CCN(C(c3ccccc3)c3ccccc3)CC2)CCN1] | 4-(4-benzhydrylpiperazine-1- carbonyl)piperazin-2-one |
| PKJ-609 | 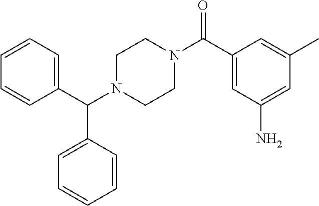 [SMILES: Cc1cc(N)cc(C(=O)N2CCN(C(c3ccccc3)c3ccccc3)CC2)c1] | (3-amino-5-methylphenyl)(4- benzhydrylpiperazin-1-yl)methanone |
| PKJ-612 | 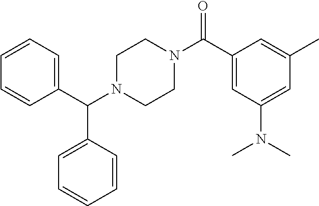 [SMILES: Cc1cc(C(=O)N2CCN(C(c3ccccc3)c3ccccc3)CC2)cc(N(C)C)c1] | (4-benzhydrylpiperazin-1-yl)(3- (dimethylamino)-5- methylphenyl)methanone |
| PKJ-606 | 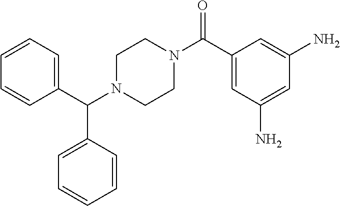 [SMILES: Nc1cc(N)cc(C(=O)N2CCN(C(c3ccccc3)c3ccccc3)CC2)c1] | (4-benzhydrylpiperazin-1-yl)(3,5- diaminophenyl)methanone |
| PKJ-607 | 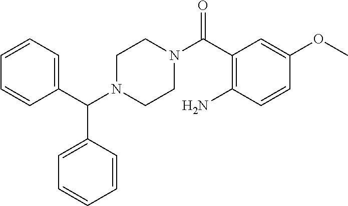 [SMILES: COc1ccc(N)c(C(=O)N2CCN(C(c3ccccc3)c3ccccc3)CC2)c1] | (2-amino-5-methoxyphenyl)(4- benzhydrylpiperazin-1-yl)methanone |
| PKJ-663 | 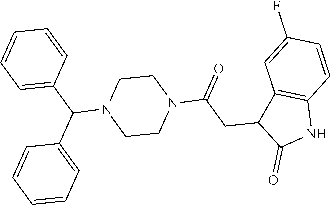 [SMILES: O=C1Nc2ccc(F)cc2C1CC(=O)N1CCN(C(c2ccccc2)c2ccccc2)CC1] | 3-(2-(4-benzhydrylpiperazin-1-yl)-2- oxoethyl)-5-fluoroindolin-2-one |
| PKJ-703 | 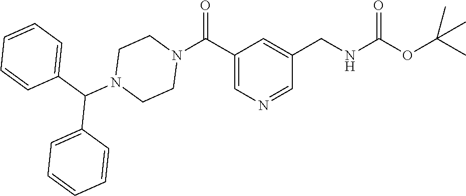 [SMILES: CC(C)(C)OC(=O)NCc1cncc(C(=O)N2CCN(C(c3ccccc3)c3ccccc3)CC2)c1] | tert-butyl ((5-(4-benzhydrylpiperazine- 1-carbonyl)pyridin-3- yl)methyl)carbamate |
| PKJ-704 | 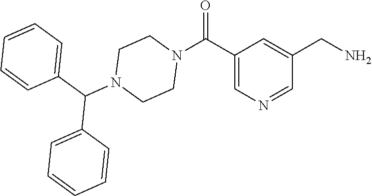 [SMILES: NCc1cncc(C(=O)N2CCN(C(c3ccccc3)c3ccccc3)CC2)c1] | (5-(aminomethyl)pyridin-3-yl)(4- benzhydrylpiperazin-1-yl)methanone |
| PKJ-710 | 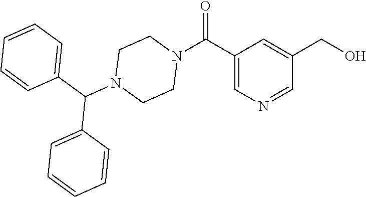 [SMILES: O=C(c1cncc(CO)c1)N1CCN(C(c2ccccc2)c2ccccc2)CC1] | (4-benzhydrylpiperazin-1-yl)(5- (hydroxymethyl)pyridin-3-yl)methanone |
| PKJ-683 | 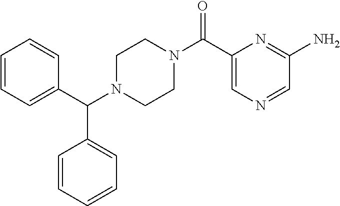 [SMILES: Nc1cncc(C(=O)N2CCN(C(c3ccccc3)c3ccccc3)CC2)n1] | (6-aminopyrazin-2-yl)(4- benzhydrylpiperazin-1-yl)methanone |
| PKJ-672 | 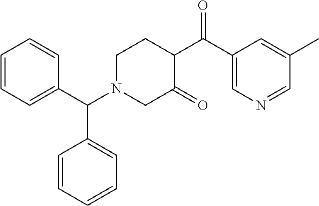 [SMILES: Cc1cncc(C(=O)C2CCN(C(c3ccccc3)c3ccccc3)CC2=O)c1] | 1-benzhydryl-4-(5- methylnicotinoyl)piperidin-3-one |
| 1 | 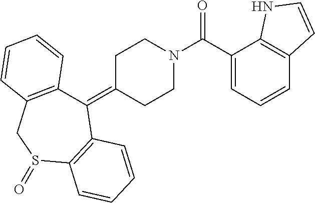 [SMILES: O=C(c1cccc2cc[nH]c12)N1CCC(=C2c3ccccc3CS(=O)c3ccccc32)CC1] | |
| 2 | 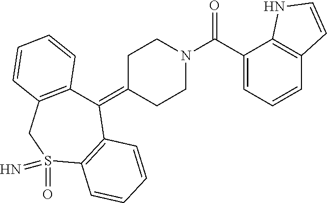 [SMILES: N=S1(=O)Cc2ccccc2C(=C2CCN(C(=O)c3cccc4cc[nH]c34)CC2)c2ccccc21] | |
| 3 | 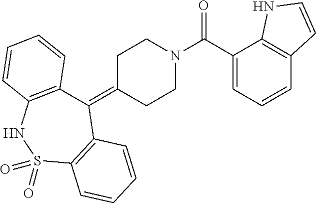 [SMILES: O=C(c1cccc2cc[nH]c12)N1CCC(=C2c3ccccc3NS(=O)(=O)c3ccccc32)CC1] | |
| 4 | 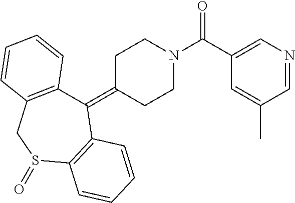 [SMILES: Cc1cncc(C(=O)N2CCC(=C3c4ccccc4CS(=O)c4ccccc43)CC2)c1] | |
| 5 | 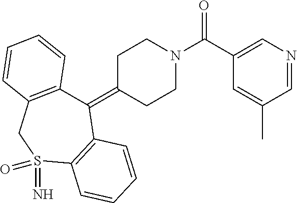 [SMILES: Cc1cncc(C(=O)N2CCC(=C3c4ccccc4CS(=N)(=O)c4ccccc43)CC2)c1] | |
| 6 | 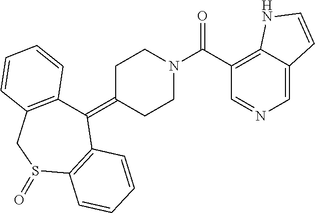 [SMILES: O=C(c1cncc2cc[nH]c12)N1CCC(=C2c3ccccc3CS(=O)c3ccccc32)CC1] | |
| 7 | 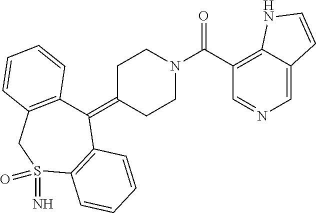 [SMILES: N=S1(=O)Cc2ccccc2C(=C2CCN(C(=O)c3cncc4cc[nH]c34)CC2)c2ccccc21] | |
| 8 | 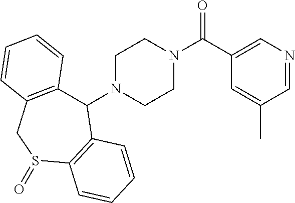 [SMILES: Cc1cncc(C(=O)N2CCN(C3c4ccccc4CS(=O)c4ccccc43)CC2)c1] | |
| 9 | 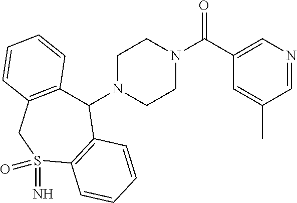 [SMILES: Cc1cncc(C(=O)N2CCN(C3c4ccccc4CS(=N)(=O)c4ccccc43)CC2)c1] | |
| 10 | 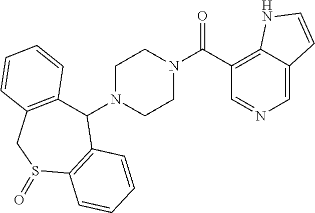 [SMILES: O=C(c1cncc2cc[nH]c12)N1CCN(C2c3ccccc3CS(=O)c3ccccc32)CC1] | |
| 11 | 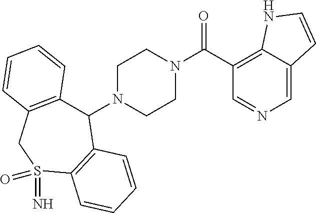 [SMILES: N=S1(=O)Cc2ccccc2C(N2CCN(C(=O)c3cncc4cc[nH]c34)CC2)c2ccccc21] | |
| 12 | 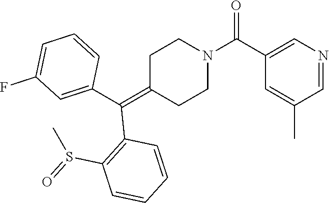 [SMILES: Cc1cncc(C(=O)N2CCC(=C(c3cccc(F)c3)c3ccccc3S(C)=O)CC2)c1] | |
| 13 | 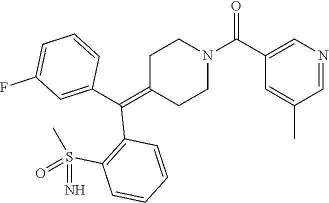 [SMILES: Cc1cncc(C(=O)N2CCC(=C(c3cccc(F)c3)c3ccccc3S(C)(=N)=O)CC2)c1] | |
| 14 | 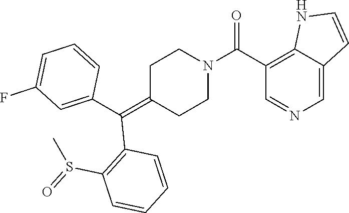 [SMILES: CS(=O)c1ccccc1C(=C1CCN(C(=O)c2cncc3cc[nH]c23)CC1)c1cccc(F)c1] | |
| 15 | 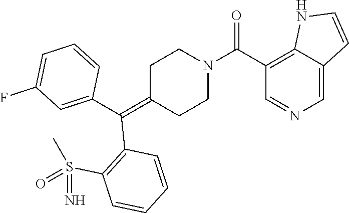 [SMILES: CS(=N)(=O)c1ccccc1C(=C1CCN(C(=O)c2cncc3cc[nH]c23)CC1)c1cccc(F)c1] | |
| 16 | 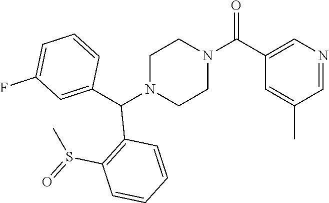 [SMILES: Cc1cncc(C(=O)N2CCN(C(c3cccc(F)c3)c3ccccc3S(C)=O)CC2)c1] | |
| 17 | 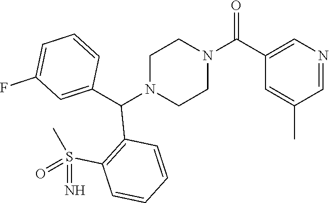 [SMILES: Cc1cncc(C(=O)N2CCN(C(c3cccc(F)c3)c3ccccc3S(C)(=N)=O)CC2)c1] | |
| 18 | 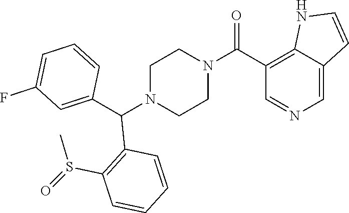 [SMILES: CS(=O)c1ccccc1C(c1cccc(F)c1)N1CCN(C(=O)c2cncc3cc[nH]c23)CC1] | |
| 19 | 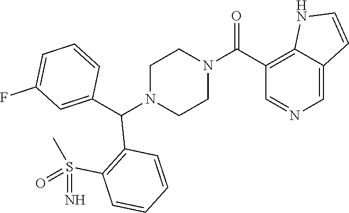 [SMILES: CS(=N)(=O)c1ccccc1C(c1cccc(F)c1)N1CCN(C(=O)c2cncc3cc[nH]c23)CC1] | |

### Methods of Use

Disclosed are methods of treating and/or preventing a coronavirus infection. These methods can comprise administering a pharmaceutical composition comprising a compound described herein and a pharmaceutically acceptable carrier or excipient; or a compound described herein. In some embodiments, the pharmaceutical composition can include a compound described herein; a CYP-3A4 inhibitor or a pharmaceutically acceptable salt, solvate, prodrug, ester, or derivative thereof; and a pharmaceutically acceptable carrier or excipient. In some embodiments, the pharmaceutical composition can include a compound described herein; ritonavir or a pharmaceutically acceptable salt, solvate, prodrug, or derivative thereof; and a pharmaceutically acceptable carrier or excipient. In some embodiments, the ritonavir or a pharmaceutically acceptable salt, solvate, prodrug, or derivative thereof can be present in the pharmaceutical composition in an amount effective to inhibit CYP-3A4 in the subject.

Suitable pharmaceutical compositions and methods of administration are described below. Also disclosed are methods of inhibiting a coronavirus main protease that comprise contacting the coronavirus with a compound described herein.

In some embodiments, the coronavirus can comprise an alphacoronavirus, a betacoronavirus, a gammacoronavirus, or a deltacoronavirus. In some embodiments, the coronavirus can comprise an avian coronavirus (IBV), porcine coronavirus HKU15 (PorCoV HKU15), Porcine epidemic diarrhea virus (PEDV), HCoV-229E, HCoV—OC43, HCoV—HKU1, HCoV-NL63, SARS-CoV, SARS-CoV-2, or MERS-CoV. In certain embodiments, the coronavirus can comprise SARS-CoV-2 (i.e., the infection can comprise Covid-19).

### Pharmaceutical Compositions and Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Also provided are pharmaceutical compositions comprising the compounds described herein (e.g., a therapeutically effective amount of a compound described herein) in combination with an excipient. Compositions, as described herein, comprising an active compound and an excipient of some sort may be useful in a variety of medical and non-medical applications. For example, pharmaceutical compositions comprising an active compound and an excipient may be useful for the treatment or prevention of an infection with a coronavirus (e.g., SARS-CoV-2).

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the compound as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, immunomodulatory agents, anticytokine, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of additional active agents include chloroquine, hydrochloroquine, Vitamin D, and Vitamin C.

### Cytochrome P450 3A4 (CYP-3A4) Inhibitor

In some embodiments, the pharmaceutical composition described herein can include the compound described herein and an inhibitor of cytochrome P450 3A4 (CYP-3A4). Administration of the inhibitor to a subject, whether prior to, concomitantly with, or after administration of the compound described herein, inhibits or reduces degradation of the compound described herein by CYP-3A4. This results in an increase in the bioavailability of the compound described herein. As a result, the compound described herein may be more effective or may be therapeutically effective at a lower dose than had it not been administered with the inhibitor (which can also result in fewer side effects or a reduction in the cost of the therapy). In some embodiments, the CYP-3A4 inhibitor can be selected from the compounds disclosed in one or more of the following patents or patent applications: U.S. Patent Publication Nos. US 2005/0209301, US 2005/0267074, US 2007/0287664, US 2010/0291034, US 2004/058982, US 2003/0235632, US 2005/0171191, U.S. Pat. Nos. 6,248,776, 6,063,809, 6,054,477, 6,162,479, 6,309,687, 6,476,066, 6,660,766, 6,124,477, 7,576,124, 5,820,915, 5,993,887, 5,990,154, 6,255,337, 6,734,313, each of which is incorporated herein by reference.

Non-limiting examples of CYP-3A4 inhibitors include ketoconazole (NIZORAL™, commercially available from Janssen Pharmaceutica), itraconazole (SPORANOX®, commercially available from Janssen-Cilag), ritonavir (NORVIR® commercially available from Abbott), nelfinavir (VIRACEPT@commercially available from Pfizer), indinavir (CRIXIVAN® commercially available from Merck & Co., Inc), erythromycin (AKNE-MYCIN®, A/T/S®, EMGEL®, ERYCETTE®, ERYDERM®, ERYGEL®, ERYMAX®, ERY-SOL®, ERYTHRA-DERM®, ETS®, STATICIN®, THERAMYCIN Z®, T-STAT®, ERYC®, ERY-TAB®, ERYTHROMYCIN BASE FILMTAB®, PCE® DISPERTAB@), clarithromycin (BIAXIN®), troleandomycin (TAO®), saquinavir, nefazodone, fluconazole, grapefruit juice, fluoxetine (PROZAC® commercially available from Eli Lilly and Company, Zoloft® commercially available from Pfizer Pharmaceuticals, ANAFRANIL® commercially available from Mallinckrodt Inc.), fluvoxamine (LUVOX®), Zyflo (ZILEUTON® commercially available from Abbott Laboratories), clotrimazole (FUNGOID® Solution, GYNE-LOTRIMIN®, GYNELOTRIMIN® 3, GYNE-LOTRIMIN® 3 Combination Pack, GYNE-LOTRIMIN®-3, LOTRIM® AF Jock Itch Cream, LOTRIMIN®, LOTRIMIN® AF, MYCELEX® TROCHE, MYCELEX®-7), midazolam (available from Apotex Corp.), naringenin, bergamottin, BAS 100 (available from Bioavailability Systems), aprepitant, atazanavir, ciprofloxacin, darunavir, diltiazem, dronedarone, imatinib, verapamil, boceprevir, cobicistat, conivaptan, itraconazole, lopinavir, mibefradil, posaconazole, telaprevir, telithromycin, voriconazole, fosamprenavir, quinupristin; as well as pharmaceutically acceptable salts, solvates, prodrugs, esters, derivatives, and combinations thereof.

In some embodiments, the CYP-3A4 inhibitor can be ritonavir or a pharmaceutically acceptable salt, solvate, prodrug, or derivative thereof. In another embodiment, the CYP-3A4 inhibitor can be ketoconazole or a pharmaceutically acceptable salt, solvate, prodrug, ester, or derivative thereof. In another embodiment, the CYP-3A4 inhibitor can be clarithromycin or a pharmaceutically acceptable salt, solvate, prodrug, ester, or derivative thereof. In another embodiment, at least one CYP-3A4 inhibitor is BAS 100 or a pharmaceutically acceptable salt, solvate, prodrug, ester, or derivative thereof. In one embodiment, at least one CYP-3A4 inhibitor is identified by the Chemical Abstracts Services (CAS) Number 684217-04-7 which corresponds to the Chemical Abstract index name 7H-Furo[3,2-g][1]benzopyran-7-one, 4-[[(2E)-5-[(4R)-4′-[[(2E)-3,7-dimethyl-2,6-octadienyl]oxy]-5,5-dimethylspiro[1,3-dioxolane-2,7′-[7H]furo[3,2-g][1]benzopyran]-4-ylj-3-methyl-2-pentenyljoxy]; the CAS Number 684217-03-6 which corresponds to the Chemical Abstract index name 7H-Furo[3,2-g][1]benzopyran-7-one, 4-[[(2E)-5-[(4R)-4′-[[2E)-6,7-dihydroxy-3,7-dimethyl-2-octenyl]oxy]-5.5-dimethylspiro[1,3-dioxolane-2,7′-[7H]furo[3,2-g][1]benzopyran]-4-yl]-3-methyl-2-pentenyl]oxy], or the CAS Number 267428-36-4 which corresponds to the Chemical Abstract index name 7H-Furo[3,2-g][1]benzopyran-7-one, 4-[[(2E)-5-[(2R,4R)-4′-[[(2E,6R)-6,7-dihydroxy-3,7-dimethyl-2-octenyl]oxy]-5,5-dimethylspiro[1,3-dioxolane-2,7′-[7H]furo[3,2-g)[1]benzopyran]-4-yl]-3-methyl-2-pentenyl]oxy], pharmaceutically acceptable salts, solvate, prodrug, ester, or derivative thereof, all of which is further described in WO 2004037827, which is incorporated by reference in its entirety.

In addition, non-limiting examples of suitable compounds that have also been identified as CYP-3A4 inhibitors are disclosed in US 2005/0209301 (at page 3, paragraph [0025] to page 5, paragraph [0071] and page 10, paragraph [0170] to page 12, paragraph [0226]) as well as US 2005/0267074 (at page 3, paragraph [0025], paragraph [0028] to page 7, paragraph [0114], page 7, paragraph to paragraph [0124], and FIGS. 1-3) incorporated herein by reference. The following is a list of specific compounds depicted in US 2005/0209301: {1-Benzyl-3-[(3-dimethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dimethylamino-ethylidene)-2-oxo-2,3-dihydro-TH-1-indole-5-sulfonyl]-isobutyl-amino}-2-hydroxy-propyl)-carbamic acid hexahydro-furo[2,3-b]furan-3-yl ester; [1-Benzyl-3-({3-[(ethyl-methyl-amino)-methylene]-2-oxo-2,3-dihydro-TH-indole-5-sulfonyl}-isobutyl-amino)-2-hydroxy-propyl]-carbamic acid hexahydro-furo[2,3-b]furan-3-yl ester; [1-Benzyl-3-({3-[1-(ethyl-methyl-amino)-ethylidene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(methyl-propyl-amino)-methylene-]-2-oxo-2,3-dihydro-TH-indole-5-sulfonyl}-amino)-propyl]-carbamic acid hexahydro-furo[2,3-b]furan-3-yl ester; [1-Benzyl-2-hydroxy-3-(isobutyl-{3-[1-(methyl-propyl-amino)-ethylidene]-2-oxo-2,3-dihydro-1H-indole-5-sulfonyl}-amino)-propyl]-carbamic acid hexahydro-furo[2,3-b]furan-3-yl ester; {1-Benzyl-3-[(3-diethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-diethylamino-ethylidene)-2-oxo-2,3-dihydro-TH-indole-5-sulfonyl]-isobutyl-amino}-2-hydroxy-propyl)-carbamic acid hexahydro-furo[2,3-b]furan-3-yl ester; {1-Benzyl-3-[(3-dipropylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dipropylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; {1-Benzyl-2-hydroxy-3-[isobutyl-(2-oxo-3-piperidin-1-ylmethylene-2,-3-dihydro-1H-indole-5-sulfonyl)amino]-propyl}-carbamic acid hexahydro-furo[2,3-b]furan-3-yl ester; (1-Benzyl-2-hydroxy-3-{isobutyl-[2-oxo-3-(1-piperidin-1-yl-ethylidene)-2,3-dihydro-TH-indole-5-sulfonyl]-amino}-propyl)-carbamic acid hexahydro-furo[2,3-b]furan-3-yl ester; {1-Benzyl-2-hydroxy-3-[isobutyl-(2-oxo-3-piperazin-1-ylmethylene-2,-3-dihydro-1H-indole-5-sulfonyl)-amino]-propyl}-carbamic acid hexahydro-furo[2,3-b]furan-3-yl ester; {1-Benzyl-2-hydroxy-3-[isobutyl-(3-morpholin-4-ylmethylene-2-oxo-2,-3-dihydro-1H-indole-5-sulfonyl)-amino]-propyl}-carbamic acid hexahydro-furo[2,3-b]furan-3-yl ester; {3-[(3-Aminomethylene-2-oxo-2,3-dihydro-TH-indole-5-sulfonyl)-isobutyl-amino]-1-benzyl-2-hydroxy-propyl}-carbamic acid hexahydro-furo[2,3-b]furan-3-yl ester; (3-{[3-(1-Amino-ethylidene)-2-oxo-2,3-dihydro-TH-indole-5-sulfonyl]-isobutyl-amino}-1-benzyl-2-hydroxy-propyl)-carbamic acid hexahydro-furo[2,3-b]furan-3-yl ester; {1-Benzyl-2-hydroxy-3-[isobutyl-(3-methylaminomethylene-2-oxo-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[3-(1-methylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-3-[(3-ethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-ethylamino-ethylidene)-2-oxo-2,3-dihydro-1H-indo-1e-5-sulfonyl]-isobutyl-amino}-2-hydroxy-propyl)-carbamic acid hexahydro-furo[2,3-b]furan-3-yl ester; [1-Benzyl-2-hydroxy-3-(isobutyl-{2-oxo-3-[(2,2,2-trifluoro-ethylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[1-(2,2,2-trifluoro-ethylamino)-ethylidene]-2,3-dihydro-1H-indole-5-sulfonyl}-amino)-propyl]-carbamic acid hexahydro-furo[2,3-b]furan-3-yl ester; [1-Benzyl-2-hydroxy-3-({3-[(2-hydroxy-ethylamino)-methylene]-2-oxo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-({3-[1-(2-hydroxy-ethylamino)-ethylidene]-2-oxo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-(isobutyl-{3-[(2-methoxy-ethylamino)-methylene]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[1-(2-methoxy-ethylamino)-ethylidene]-2-oxo-2,3-dihydro-1H-indole-5-sulfonyl}-amino)-propyl]-carbamic acid hexahydro-furo[2,3-b]furan-3-yl ester; [1-Benzyl-3-({3-[(2-dimethylamino-ethylamino)-methylene]-2-oxo-2,3-dihydro-TH-indole-5-sulfonyl}-isobutyl-amino)-2-hydroxy-propyl]-carbamic acid hexahydro-furo[2,3-b]furan-3-yl ester; [1-Benzyl-3-({3-[1-(2-dimethylamino-ethylamino)-ethylidene]-2-oxo-2-,3-dihydro-1H-indole-5-sulfonyl}-isobutyl-amino)-2-hydroxy-propyl]-carbamic acid hexahydro-furo[2,3-b]furan-3-yl ester; (1-Benzyl-2-hydroxy-3-{isobutyl-[3-(isopropylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isopropylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-propylaminomethylene-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(1-propylamino-ethylidene)-2,3-dihydro-TH-indole-5-sulfonyl]-amino}-propyl)-carbamic acid hexahydro-furo[2,3-b]furan-3-yl ester; {1-Benzyl-2-hydroxy-3-[isobutyl-(2-oxo-3-pyrrolidin-2-ylidene-2,3-dihydro-1H-indole-5-sulfonyl)-amino]-propyl}-carbamic acid hexahydro-furo[2,3-b]furan-3-yl ester; {1-Benzyl-3-[(3-butylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-butylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-2-hydroxy-3-{isobutyl-[3-(isobutylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isobutylamino-ethylidene)-2-oxo-2,3-dihydro-1H-indole-5-sulfonyl]-amino}-propyl)-carbamic acid hexahydro-furo[2,3-b]furan-3-yl ester; (1-Benzyl-3-{[3-(tert-butylamino-methylene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-3-{[3-(1-tert-butylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; [1-Benzyl-3-({3-[(2,2-dimethyl-propylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(2,2-dimethyl-propylamino)-ethylidene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(2-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[(3-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-3-({3-[(3,3-dimethyl-butylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(1-isopropyl-2-methyl-propylamino)-methylene]-2-oxo-2,3-dihydro-1H-indole-5-sulfonyl}-amino)-propyl]-carbamic acid hexahydro-furo[2,3-b]furan-3-yl ester; {1-Benzyl-2-hydroxy-3-[isobutyl-(2-oxo-3-phenylaminomethylene-2,3-dihydro-1H-indole-5-sulfonyl)-amino]-propyl}-carbamic acid hexahydro-furo[2,3-b]furan-3-yl ester; (1-Benzyl-3-{([3-(benzylamino-methylene)-2-oxo-2,3-dihydro-1H-indole-5-sulfonyl]-isobutyl-amino}-2-hydroxy-propyl)-carbamic acid hexahydro-furo[2,3-b]furan-3-yl ester; (1-Benzyl-3-{[3-(1-benzylamino-ethylidene)-2-oxo-2,3-dihydro-TH-indole-5-sulfonyl]-isobutyl-amino}-2-hydroxy-propyl)-carbamic acid hexahydro-furo[2,3-b]furan-3-yl ester; [1-Benzyl-3-({3-[(cyclohexylmethyl-amino)-methylene]-2-oxo-2,3-dihydro-1H-indole-5-sulfonyl]-isobutyl-amino)-2-hydroxy-propyl}-carbamic acid hexahydro-furo[2,3-b]furan-3-yl ester; {1-Benzyl-2-hydroxy-3-[isobutyl-(2-oxo-3-{[(pyridin-4-ylmethyl)-amino]-methylene}-2,3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(phenethylamino-methylene)-2,3-dihydro-1H-indole-5-sulfonyl]-amino}-propyl)-carbamic acid hexahydro-furo[2,3-b]furan-3-yl ester; [1-Benzyl-3-({3-[(2-cyclohex-1-enyl-ethylamino)-methylene]-2-oxo-2,-3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{2-oxo-3-[(2-pyridin-2-yl-ethylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(2-phenyl-propylamino)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(4-phenyl-butylamino)-methylene]-2,3-dihydro-1H-indole-5-sulfonyl}-amino)-propyl]-carbamic acid hexahydro-furo[2,3-b]furan-3-yl ester; {1-Benzyl-2-hydroxy-3-[isobutyl-(3-nonylaminomethylene-2-oxo-2,3-dihydro-1H-indole-5-sulfonyl)-amino]-propyl}-carbamic acid hexahydro-furo[2,3-b]furan-3-yl ester; and (1-Benzyl-2-hydroxy-3-{[3-(1-hydroxy-ethylidene)-2-oxo-2,3-dihydro-1H-indole-5-sulfonyl]-isobutyl-amino}-propyl)-carbamic acid hexahydro-furo[2,3-b]furan-3-yl ester; and the pharmaceutically acceptable salts thereof, as single stereoisomers or mixtures of stereoisomers, solvate, prodrug, ester, or derivative thereof. Likewise, see FIGS. 1A-1G for a list of specific compounds depicted in US 2005/0267074. Notably, US 2005/0267074 emphasizes that compounds having a benzofuran moiety are potent inhibitors of CYP-3A4.

In addition, non-limiting examples of suitable compounds that have also been identified as CYP-3A4 inhibitors are disclosed in US 2014/0037574 incorporated herein by reference. The following is a list of specific compounds depicted in US 2005/0209301: poly(ethylene glycol), methoxy poly(ethylene glycol), aminated poly(ethylene glycol), 0-(2-aminoethyl)-O-methoxy poly(ethylene glycol), polyoxyethylene glycol, branched poly(ethylene glycol), 3-arm poly(ethylene glycol), 4-arm poly(ethylene glycol), 8-arm-poly(ethylene glycol)polyamine, poly(L-lysine), poly(L-arginine), poly(L-alanine), poly(L-valine), poly(L-serine), poly(L-histidine), poly(L-isoleucine), poly(L-leucine), poly(L-glutamic acid), poly(L-glutamine), poly(L-guanidine), poly(methyl methacrylate), polyvinyl acetate, polyacrylate, poly(lactic-co-glycolic acid), pharmaceutically acceptable salts, solvate, prodrug, ester, or derivative thereof.

In some embodiments, the CYP-A4 inhibitor can be poly(ethylene glycol) or a derivative thereof, such as of methoxy poly(ethylene glycol) having a molecular weight in the range of about 500 to about 10 000 g/mol, aminated poly(ethylene glycol) having a molecular weight in the range of about 500 to about 10 000 g/mol, O-(2-aminoethyl)-O-methoxy poly(ethylene glycol) having a molecular weight of about 7500 g/mol, polyoxyethylene glycol having a molecular weight in the range of about 500 to about 10000 g/mol, branched poly(ethylene glycol) having a molecular weight in the range of about 500 to about 25000 g/mol, 3-arm poly(ethylene glycol), 4-arm poly(ethylene glycol) having a molecular weight in the range of about 10 000 to about 20 000 g/mol or 8-arm-poly(ethylene glycol) having a molecular weight in the range of about 10 000 g/mol to about 40 000 g/mol, and more particularly in the range of about 10 000 to 20 000 g/mol.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Identification of Inhibitors of SARS-CoV-2 Main Protease (Mpro)

Small molecule compounds designed based on structures of inhibitor complexes with SARS-CoV-2 main protease (Mpro) are described and evaluated. Briefly, a diverse small molecule library containing 113,392 compounds was screened against the SARS-CoV-2 main protease (Mpro). One of the hits was a compound that included a diphenylmethyl piperazine core structure. This his molecule exhibited an enzymatic IC50 of 1.4 μM. Modification of this molecule led to the synthesis of numerous analogs, with the most potent analogs designed based on the structure of inhibitor complexes with Mpro. In one example, a potent derivative resulted from the removal of a para-chloro substituent on one of the phenyl groups. This modification reduced the IC50 to 0.48 μM. This compound showed anti-viral activity on a SARS-CoV-2 infected HeLa-ACE2 cell line with an EC50 of 1.0 μM while not showing overt cytotoxicity against the same cell line up to 40 μM (CC50>40 μM) or human dermal fibroblasts up to 125 μM (CC50>125 μM). These compounds can be used as therapeutic agents (anti-viral agents) for the treatment of COVID-19.

### General Experimental Methods

Mpro HTS assay: An HTS assay was adapted from procedures reported in the literature. Briefly, the Mpro HTS assay used 400 nM enzyme, 50 uM acetyl-TSAVLQ-AMC fluorogenic substrate (Km=60 uM), and 20 uM library compound. Each compound was first incubated with enzyme at room temperature for 30 mins, and the enzymatic reaction was initiated via the addition of fluorogenic peptide substrate. Using an endpoint readout after 1 hr of catalysis at room temperature, the AMC fluorescence intensity excitation of 350 nM and emission of 460 nM (Ex/Em 350/460 nm) was measured for each well.

SARS-CoV-2 viral replication assay: All verified hits defined using the Mpro HTs assay were tested in a viral replication assay at 25 uM. Hits were acoustically transferred into 384-well plates and HeLa-ACE2 cells were seeded in the plates at a density of 1.0×103 cells per well. In the BSL3 facility, SARS-CoV-2 (strain USA-WA1/2020) was propagated in Vero E6 cells and the plates were diluted in assay media to achieve ˜30-50% infected cells. Plates were incubated for 24 h at 34° C. 5% CO₂, and then fixed with 8% formaldehyde. Fixed cells were stained with human polyclonal sera as the primary antibody, goat anti-human H+L conjugated Alexa 488 as the secondary antibody, and antifade-46-diamidino-2-phenylindole (DAPI) to stain DNA. Plates were imaged using the ImageXpress Micro Confocal High-Content Imaging System (Molecular Devices) with a 10× objective, with 4 fields imaged per well. Images were analyzed using the Multi-Wavelength Cell Scoring Application Module (MetaXpress), with DAPI staining identifying the host-cell nuclei (the total number of cells in the images) and the SARS-CoV-2 immunofluorescence signal leading to identification of infected cells. Actives from the screen were tested in a dose-response assay.

Uninfected host cell cytotoxicity counter screen: HeLa-ACE2 cells were maintained as described for the infection assay and seeded in the assay-ready plates at 400 cells/well. Plates were incubated for 24 hours at 37° C. 5% CO₂. To assess cell viability, the Image-iT DEAD green reagent was used according to manufacturer instructions. Cells were fixed with 4% paraformaldehyde, and counterstained with DAPI. Fixed cells are imaged as described above.

Viral replication data analysis: Data from the primary screen and the host cell cytotoxicity screen are normalized to neutral (DMSO) minus inhibitor controls (2.5 μM Remdesivir for antiviral effect and 10 μM puromycin for infected host cell toxicity). For the uninfected host cell cytotoxicity counter-screen 40 μM puromycin is used as the positive control. Concentration response experiments will be tested in triplicate.

### Synthetic Methods

It will be apparent to one skilled in the art that the compounds described herein can be prepared by a variety of synthetic routes, including but not limited to substitution of appropriate reagents, solvents or catalyst, change of reaction sequence, and variation of protecting groups.

General Procedure A. Diphenylmethyl piperazine derivatives were prepared according to General Procedure (A) described in scheme 1 below.

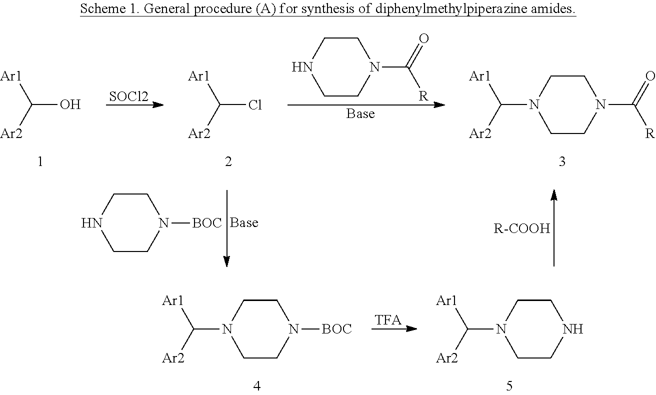

Briefly, a di-aromatic methanol starting material (1) was converted to a di-aromatic methyl chloride (2) with thionyl chloride or similar reagents. Reaction of the di-aromatic methyl chloride (2) with a corresponding piperazine amide in organic solvent, such as DMF, DMSO, THF, DCM, dioxane, acetonitrile, ethanol in the presence or absence of a catalyst, such as KI, at a temperature range from 5° C. to 150° C. in the presence of base, such as TEA, K₂CO₃. Li₂CO₃, LiOH, or KOH, to give a product di-aromatic methyl piperazine amide (3).

Product amide (3) was also prepared by an alternative method. Reaction of di-aromatic methyl chloride (2) with a Boc-protected-piperazine in organic solvent, such as DMF, DMSO, THF, DCM, dioxane, acetonitrile, or ethanol in the presence or absence of a catalyst, such as KI, at a temperature range from 5° C. to 150° C. in the presence of base, such as TEA, K₂CO₃. Li₂CO₃, LiOH, or KOH, give a Boc-protected di-aromatic methyl piperazine (4). De-protection of (4) with TFA or HCl in organic solution gave a di-aromatic methyl piperazine (5). Coupling of piperazine (5) with a corresponding acid in the presence of coupling reagents, such as T3P, EDCI, CDI or via acyl chloride in organic solvent, such as DCM, THF, DMF, produces amide (3).

If di-aromatic methanol (1) is not commercially available, it was synthesized or could be prepared with known methods, such as, reduction of di-aromatic ketone with NaBH₄, reaction of an aromatic aldehyde with an aromatic magnesium bromide or an aromatic bromide/iodide in the presence of BuLi.

The following compounds were prepared according to General Procedure (A): AA-551, AA-557, AA-558, AA-559, AA-560, AA-564, AA-565, AA-566, AA-567, AA-570, AA-571, AA-572, AA-573, AA-575, AA-577, AA-578, AA-579, AA-582, AA-583, AA-589, AA-590, AA-591, AA-592, AA-594, AA-595, AA-596, AA-597, AA-598, AA-599, AA-602, AA-603, AA-604, AA-605, AA-606, AA-607, AA-608, AA-611, AA-612, AA-613, AA-614, AA-616, AA-620, AA-624, AA-625, AA-627, AA-630, AA-633, AA-637, AA-638, AA-642, AA-643, MKP-1151, MKP-1152, MKP-1157, MKP-1157, MKP-1158, MKP-1167, MKP-1168, MKP-1183, MKP-1188, MKP-1189, MKP-1192, MKP-1193, MKP-1194, MKP-1195, MKP-1200, MKP-1203, MKP-1204, MKP-1205, MKP-1215, MKP-1217, MKP-1219, MKP-1220, MKP-1221, MKP-1222, MKP-1225, MKP-1227, MKP-1229, MKP-1230, MKP-1268, MKP-1272, MKP-1276, MKP-1277, MKP-1280, MKP-1287, MKP-1290, MKP-1304, MKP-1309, MKP-1318, MKP-1319, NZ-769, NZ-797, NZ-799, NZ-800 and ZS-325.

General Procedure (B). Scheme 2 (below) describes General Procedure (B), which was used for the synthesis of diphenylmethylpiperazin sulfonyl amides.

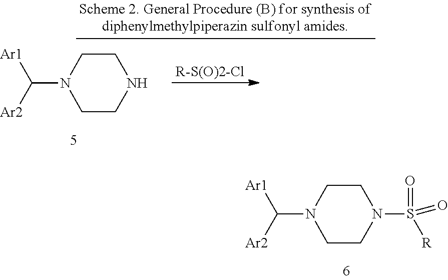

Briefly, the piperazine sulfonyl amides (6) were synthesized by condensation of diphenylmethylpiperazine (5) with an aromatic sulfonyl chloride in organic solvent, such as DMF, DCM, pyridine, acetonitrile in the presence of base, such as TEA, pyridine, or K₂CO₃.

The following compounds were prepared according to General Procedure (B): MKP-1161, MKP-1162, and MKP-1176.

General Procedure (C). Scheme 3 below outlines General Procedure (C).

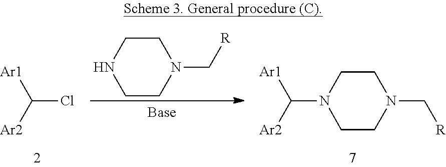

Briefly, reaction of di-aromatic methyl chloride (2) with a corresponding piperazine in organic solvent, such as DMF, DMSO, THF, DCM, dioxane, acetonitrile, ethanol in the presence or absence of a catalyst, such as KI, at a temperature range from 5° C. to 150° C. in the presence of base, such as TEA, K₂CO₃. Li₂CO₃, LiOH, KOH, gave a di-aromatic methyl piperazine product (7).

The following compounds were prepared according to the General Procedure (C): MKP-1306, and MKP-1307.

General Procedure (D). Diphenylmethyl piperidine and Diphenylmethylidene piperidine derivatives were prepared using General Procedure (D), which is outlined in Scheme 4 below.

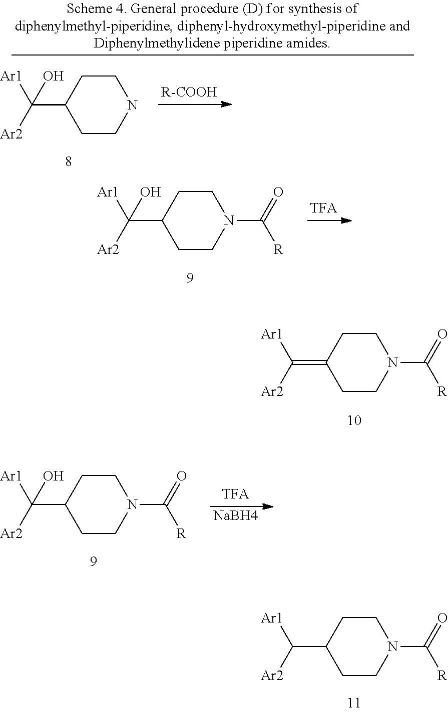

The products diphenyl-hydroxymethyl-piperidine (9) were prepared by coupling of piperidine (8) with a corresponding acid in the presence of coupling reagents, such as T3P, EDCI, CDI in organic solvent, such as DCM, THF, DMF. Treatment of diphenyl-hydroxymethyl-piperidine (9) with TFA gave a diphenylmethylidene piperidine amide (10). Treatment of diphenyl-hydroxymethyl-piperidine (9) with TFA in the presence of reducing reagent NaBH₄ gave a diphenylmethyl piperidine amide (11).

The following compounds were prepared according to the General Procedure (D): NZ-770, NZ-771, NZ-772, NZ-778, NZ-779, NZ-780, NZ-781, NZ-782, NZ-783, NZ-786, NZ-788, NZ-789, NZ-790, NZ-791, and NZ-792.

General Procedure (E). Tricyclo[9.4.0.03,8]pentadeca-1(11),3(8),4,6,12,14-hexaen piperazine derivatives were prepared according to General procedures:

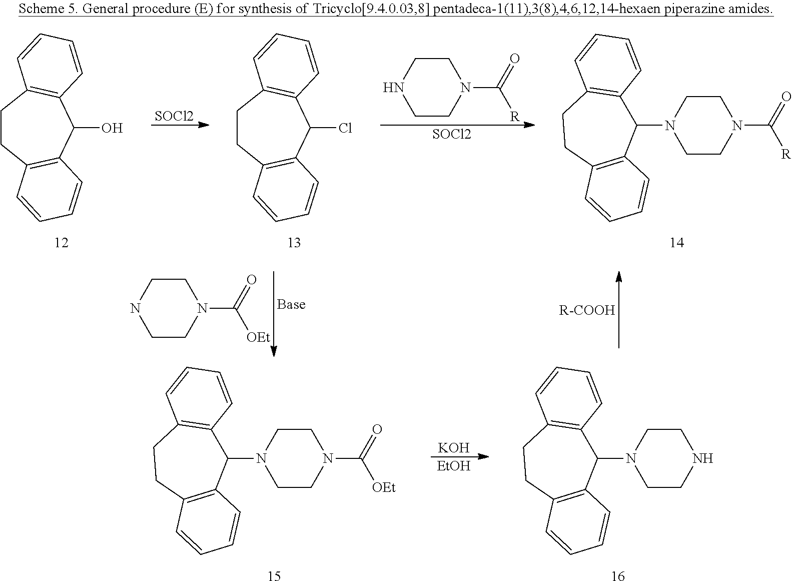

Briefly, the starting material tricyclic hydroxy (12) was converted to a tricyclic chloride (13) with thionyl chloride or similar reagents. Reaction of tricyclic chloride (13) with a corresponding piperazine amide in organic solvent, such as DMF, DMSO, THF, DCM, dioxane, acetonitrile at a temperature range from 5° C. to 150° C. in the presence of base, such as TEA, K₂CO₃. Li₂CO₃, LiOH, KOH, gave a product tricyclic piperazine amide (14).

Product amide (14) was also prepared by alternative methods. Reaction of tricyclic chloride (13) with an ethyl piperazine-1-carboxylate in organic solvent, such as DMF, DMSO, THF, DCM, dioxane, acetonitrile, at a temperature range from 5° C. to 150° C. in the presence of base, such as TEA, K₂CO₃. Li₂CO₃, LiOH, KOH, gave a tricyclic piperazine carbamate (15). Hydrolysis of (15) with KOH in ethanol gave a tricyclic piperazine (16). Coupling of piperazine (16) with a corresponding acid in the presence of coupling reagents, such as T3P, EDCI, CDI or via acyl chloride in organic solvent, such as DCM, THF, DMF, produces a tricyclic piperazine amide (14).

By the similar methods, tricyclo[9.4.0.03,8]pentadeca-1(11),3(8),4,6,12,14-hexaen (III), tricyclo[9.4.0.03,8]pentadeca-1(11),3(8),4,6,9,12,14-heptaen (IV), 6,11-dihydrobenzo[c][1]benzoxepin (V), 6,11-dihydrobenzo[c][1]benzothiepin(VI), 5,5-dioxo-6,11-dihydrobenzo[c][1]benzothiepin(VII), and 6-oxo-5,11-dihydrobenzo[c][1]benzazepin(VIII) piperazine amide derivatives were prepared.

The following compounds were prepared according to the General Procedure (E): NZ-773, NZ-774, NZ-776, NZ-784, NZ-785, NZ-795, NZ-796, NZ-798, ZS-312, ZS-316, ZS-317, ZS-318, ZS-319, ZS-320, ZS-324, ZS-329, ZS-331, ZS-333, ZS-335, ZS-337, ZS-338, ZS-339, ZS-342, ZS-346, ZS-347, ZS-355, and ZS-361.

General Procedure (F). Scheme 6 below outlines General Procedure (F), which was used for the synthesis of 4-(2-tricyclo[9.4.0.03,8]pentadeca-1(11),3(8),4,6,12,14-hexaenylidene)piperidine amides.

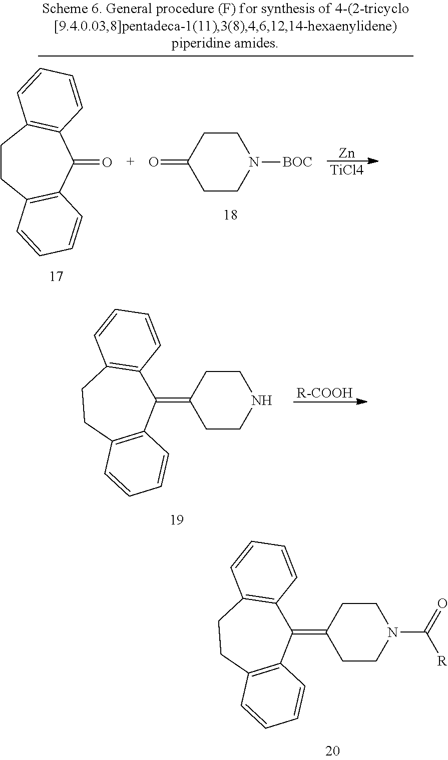

Briefly, the tricyclic ketone (17) was reacted with 1-Boc-4-piperidone (18) in organic solvent, such as dioxane, THF, toluene, in the presence of Zn and TiCl4 to give a 4-(2-tricyclo[9.4.0.03,8]pentadeca-1(11),3(8),4,6,12,14-hexaenylidene)piperidine (19).

Coupling of piperidine (19) with a corresponding acid in the presence of coupling reagents, such as T3P, EDCI, CDI or via acyl chloride in organic solvent, such as DCM, THF, DMF, produces a tricyclic piperidine amide (20).

By the similar methods, 4-(2-tricyclo[9.4.0.03,8]pentadeca-1(11),3(8),4,6,12,14-hexaenylidene)piperidine (III), 4-(2-tricyclo[9.4.0.03,8]pentadeca-1(11),3(8),4,6,9,12,14-heptaenylidene)piperidine (IV), 4-(6H-benzo[c][1]benzoxepin-11-ylidene)piperidine (V),

4-(6H-benzo[c][1]benzothiepin-11-ylidene)piperidine (VI), 11-(4-piperidylidene)-6H-benzo[c][1]benzothiepine 5,5-dioxide(VII), 11-(4-piperidylidene)-5H-benzo[c][1]benzazepin-6-one (VIII) amide derivatives were prepared.

The following compounds were prepared according to the general procedure F: NZ-793, NZ-794, NZ-801, NZ-802, NZ-803, NZ-804, NZ-805, ZS-313, and ZS-336.

General Procedure (G). Scheme 7 below outlines General Procedure (G),

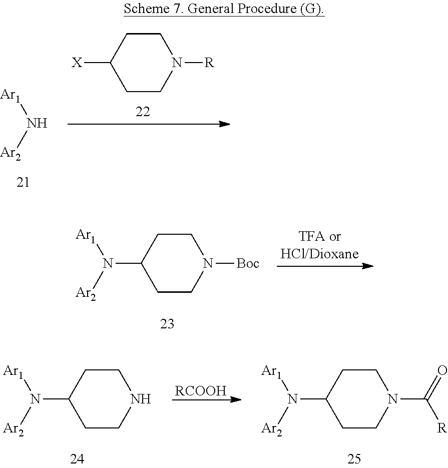

Briefly, reaction of diaryl amine (21) with a corresponding piperadine chloride in organic solvent, such as DMF, DMSO, THF, DCM, dioxane, acetonitrile, ethanol in the presence or absence of a catalyst, such as KI, at a temperature range from 5° C. to 150° C. in the presence of base, such as TEA, K₂CO₃, LiOH, KOH, gave a di-aromatic methyl piperadine product (23).

The following compounds were prepared according to the General Procedure (G): AA-612.

General Procedure (H). Scheme 8 below outlines General Procedure (H),

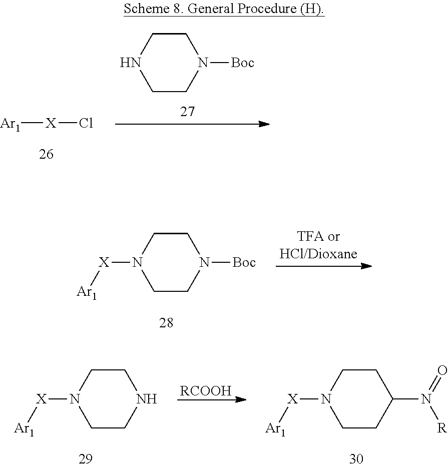

### X=CH₂, CHAr1, or CO

Briefly, reaction of mono or diaryl chloride (26) with a corresponding Boc-protected piperidine in organic solvent, such as DMF, DMSO, THF, DCM, dioxane, acetonitrile, in the presence or absence of a catalyst, such as KI, at a temperature range from 5° C. to 150° C. in the presence of base, such as TEA, K₂CO₃, LiOH, KOH, gave a mono or di-aromatic methylene piperidine or piperidine amide product (28). De-protection of (28) with TFA or HCl in organic solution gave a di-aromatic methyl piperadine (29). Coupling of piperadine (29) with a corresponding acid in the presence of coupling reagents, such as T3P, EDCI, CDI or via acyl chloride in organic solvent, such as DCM, THF, DMF, produces amide (30).

The following compounds were prepared according to the General Procedure (H): AA-580, AA-581, and AA-588.

### Results

The activity of example compounds was evaluated using the test methods described above. Details of the activity of example compounds are included in the Table 2 and Table 3 below.

TABLE 2

| | | CoV-2 | HeLa-ACE2 | Uninfected | CoV-2 |
|---|---|---|---|---|---|
| | Enzyme | HeLa | Total Cells | HeLa-ACE2 | Vero |
| | IC50 | EC50 | EC50 | CC50 | EC50/100 |
| Name | [uM] | [uM] | [uM] | [uM] | [ug/ml] |
| AA-551 | 0.22 | 0.82 | >9.61 | >39.84 | 2.5 |
| AA-557 | NA | | | | |
| AA-558 | NA | | | | |
| AA-559 | NA | | | | |
| AA-560 | NA | | | | |
| AA-563 | NA | | | | |
| AA-564 | NA | | | | |
| AA-565 | NA | | | | |
| AA-567 | NA | | | | |
| AA-569 | NA | | | | |
| AA-570 | NA | | | | |
| AA-571 | NA | | | | |
| AA-572 | NA | | | | |
| AA-573 | NA | | | | |
| AA-575 | NA | | | | |
| AA-577 | 8.3 | 8.51 | >9.61 | >39.84 | |
| AA-578 | NA | | | | |
| AA-579 | 7.7 | >9.61 | >9.61 | >39.84 | |
| AA-580 | NA | | | | |
| AA-581 | NA | | | | |
| AA-582 | 60% @ | | | | |
| | 20 uM | | | | |
| AA-583 | 0.62 | 1.53 | >9.61 | >39.84 | |
| AA-589 | 0.48 | 1.43 | >9.61 | >39.84 | |
| AA-590 | 0.99 | 4.9 | >9.61 | >39.84 | |
| AA-591 | 0.3 | 0.89 | >9.61 | >39.84 | |
| AA-592 | 0.97 | 2.72 | >9.61 | >39.84 | |
| AA-594 | 0.09 | 0.52 | >9.61 | >39.84 | 2.5 |
| AA-595 | 0.41 | 0.81 | >9.61 | >39.84 | |
| AA-596 | 0.13 | 0.36 | >9.61 | >39.84 | 2.5 |
| AA-597 | 3.9 | >9.61 | >9.61 | >39.84 | |
| AA-598 | 1.9 | >9.61 | >9.61 | >39.84 | |
| AA-599 | 1.6 | 6.03 | >9.61 | >39.84 | |
| AA-600 | NA | 4.15 | >9.61 | >39.84 | |
| AA-602 | NA | >9.606 | >9.606 | >39.84 | NA |
| AA-603 | NA | >9.61 | >9.61 | >39.84 | |
| AA-604 | 0.081 | 0.24 | >9.61 | >39.84 | 5 |
| AA-605 | 1.3 | 3.033 | 7.165 | >39.84 | |
| AA-606 | >10 | >9.61 | >9.61 | >39.84 | |
| AA-607 | NA | | | | |
| AA-608 | NA | 1.737 | 0.863 | >39.84 | |
| AA-611 | 2.5 | >9.61 | >9.61 | >39.84 | |
| AA-612 | NA | 1.737 | 0.863 | >39.84 | |
| AA-613 | 0.82 | >9.61 | >9.61 | >39.84 | |
| AA-614 | NA | >9.61 | >9.61 | >39.84 | |
| AA-616 | NA | >9.61 | >9.61 | >39.84 | |
| AA-620 | 0.26 | 0.717 | 0.841 | >39.84 | |
| AA-624 | 0.89 | 2.618 | >9.61 | >39.84 | |
| AA-625 | 0.033 | 0.072 | 0.085 | 26.466 | |
| AA-627 | 0.048 | 2.998 | >9.61 | >39.84 | NA |
| AA-630 | 1.37 | 7.730 | 8.565 | >39.84 | |
| AA-633 | 0.64 | 1.583 | 2.768 | >39.84 | |
| AA-637 | 0.09 | >9.61 | >9.61 | >39.84 | |
| AA-638 | 0.22 | 0.893 | 0.711 | >39.84 | |
| AA-642 | 0.21 | 1.052 | 3.238 | >39.84 | |
| AA-643 | 0.058 | 0.348 | 1.048 | 21.521 | |
| AA-645 | NA | >9.61 | >9.61 | >39.84 | |
| AA-646 | 6.1 | 5.197 | >9.61 | >39.84 | |
| AA-649 | NA | >9.61 | >9.61 | >39.84 | |
| AA-652 | NA | | | | |
| AA-654 | NA | | | | |
| AA-655 | NA | | | | |
| AA-658 | 0.8 | 1.952 | >9.61 | >39.84 | |
| AA-660 | NA | | | | |
| AA-661 | NA | | | | |
| AA-663 | 0.038 | | | | |
| AA-664 | NA | | | | |
| AA-665 | NA | | | | |
| AA-666 | NA | | | | |
| AA-667 | 3.32 | | | | |
| MKP-1147 | NA | | | | |
| MKP-1151 | NA | | | | |
| MKP-1152 | NA | | | | |
| MKP-1157 | NA | | | | |
| MKP-1157 | NA | | | | |
| MKP-1158 | NA | | | | |
| MKP-1161 | NA | | | | |
| MKP-1162 | NA | | | | |
| MKP-1167 | NA | >9.61 | >9.61 | >39.84 | |
| MKP-1168 | NA | | | | |
| MKP-1171 | NA | | | | |
| MKP-1175 | NA | | | | |
| MKP-1176 | NA | | | | |
| MKP-1183 | 1.7 | 4.34 | >9.61 | >39.84 | |
| MKP-1188 | 50% inh | | | | |
| | @ 20 | | | | |
| | uM | | | | |
| MKP-1189 | 60% inh | | | | |
| | @ 20 | | | | |
| | uM | | | | |
| MKP-1192 | 1.93 | 4.83 | >9.61 | >39.84 | |
| MKP-1193 | 0.45 | 0.81 | >9.61 | >39.84 | |
| MKP-1194 | 1.1 | 2.12 | >9.61 | >39.84 | |
| MKP-1195 | 2.68 | 8.77 | >9.61 | >39.84 | |
| MKP-1200 | 0.32 | | | | |
| MKP-1203 | 2.8 | | | | |
| MKP-1204 | 2.3 | 6.66 | >9.61 | >39.84 | |
| MKP-1205 | 0.55 | | | | |
| MKP-1215 | 4.5 | 2.81 | >9.61 | >39.84 | |
| MKP-1217 | 0.32 | 0.475 | 1.309 | >39.84 | 10 |
| MKP-1219 | NA | >9.606 | >9.606 | >39.84 | |
| MKP-1220 | 0.25 | 0.884 | 2.637 | >39.84 | |
| MKP-1221 | 0.33 | 0.831 | 0.556 | >39.84 | |
| MKP-1222 | 0.37 | 1.403 | 1.242 | >39.84 | |
| MKP-1225 | 0.038 | 0.210 | 0.481 | >39.84 | 2.5 |
| MKP-1227 | 0.088 | 0.457 | 0.614 | >39.84 | |
| MKP-1229 | NA | >9.606 | >9.606 | >39.84 | |
| MKP-1230 | 1.3 | 2.801 | >9.61 | >39.84 | |
| MKP-1268 | 0.036 | 0.111 | 0.143 | >39.84 | 2.5 |
| MKP-1272 | 0.365 | 0.246 | 0.117 | 33.621 | 10 |
| MKP-1276 | 0.211 | 0.637 | 3.411 | 22.911 | |
| MKP-1277 | 0.15 | 0.747 | 2.697 | >39.84 | |
| MKP-1280 | 0.061 | 0.158 | 0.261 | >39.84 | |
| MKP-1287 | 0.066 | 0.599 | 0.810 | >39.84 | |
| MKP-1290 | 0.096 | 0.105 | 0.186 | >39.84 | |
| MKP-1304 | NA | | | | |
| MKP-1306 | NA | >9.61 | >9.61 | >39.84 | |
| MKP-1307 | NA | >9.61 | >9.61 | >39.84 | |
| MKP-1309 | NA | >9.61 | >9.61 | >39.84 | |
| MKP-1318 | NA | >9.61 | >9.61 | >39.84 | |
| MKP-1319 | NA | | | | |
| MKP-1322 | NA | >9.61 | >9.61 | >39.84 | |
| MKP-1323 | NA | >9.61 | >9.61 | 31.696 | |
| MKP-1324 | NA | >9.61 | >9.61 | >39.84 | |
| MKP-1332 | NA | >9.61 | >9.61 | >39.84 | |
| MKP-1335 | NA | | | | |
| MKP-1338 | NA | | | | |
| MKP-1344 | NA | | | | |
| MKP-1349 | 0.042 | | | | |
| MKP-1355 | 0.028 | | | | |
| MKP-1359 | 0.044 | | | | |
| MKP-1360 | 0.035 | | | | |
| MKP-1371 | 0.025 | | | | |
| MKP-1381 | 0.094 | | | | |
| MKP-1392 | 0.093 | | | | |
| MKP-1398 | 0.58 | | | | |
| MKP-1400 | 0.074 | | | | |
| MKP-1404 | 2.66 | | | | |
| MKP-1410 | 0.041 | | | | |
| MKP-1416 | 0.176 | | | | |
| MKP-1421 | 0.92 | | | | |
| MKP-1422 | 0.11 | | | | |
| MKP-1440 | 0.17 | | | | |
| MKP-1442 | 1.6 | | | | |
| MKP-1445 | NA | | | | |
| MKP-1449 | NA | | | | |
| MKP-1450 | NA | | | | |
| MKP-1451 | NA | | | | |
| NZ-769 | 11.3 | 8.05 | >9.61 | >39.84 | |
| NZ-770 | 0.38 | 0.51 | >9.61 | >39.84 | |
| NZ-771 | 0.25 | 0.94 | >9.61 | >39.84 | 20 |
| NZ-772 | 0.32 | 1.01 | >9.61 | >39.84 | |
| NZ-773 | 0.1 | 0.7 | >9.61 | >39.84 | 20 |
| NZ-774 | 0.13 | 0.63 | >9.61 | >39.84 | 5 |
| NZ-776 | 0.14 | 0.48 | >9.61 | >39.84 | 2.5 |
| NZ-778 | 1.7 | 3.63 | >9.61 | >39.84 | |
| NZ-779 | 0.37 | 0.71 | >9.61 | >39.84 | |
| NZ-780 | 0.31 | 1.39 | >9.61 | 30.1 | |
| NZ-781 | 0.11 | 1.05 | >9.61 | 35.76 | |
| NZ-782 | 0.23 | 0.57 | >9.61 | >39.84 | 5 |
| NZ-783 | 0.085 | 0.57 | >9.61 | 25.6 | 10 |
| NZ-784 | 0.088 | 0.36 | >9.61 | 39.4 | NA |
| NZ-785 | 0.066 | 0.21 | >9.61 | 24.74 | 2.5 |
| NZ-786 | 0.17 | 0.57 | >9.61 | 28.5 | 10 |
| NZ-788 | NA | >9.606 | >9.606 | >39.84 | |
| NZ-789 | NA | >9.606 | >9.606 | >39.84 | |
| NZ-790 | NA | >9.606 | >9.606 | >39.84 | |
| NZ-791 | NA | >9.606 | >9.606 | >39.84 | |
| NZ-792 | >10 | >9.61 | >9.61 | >39.84 | |
| NZ-793 | 0.12 | 0.527 | 0.520 | 33.059 | 5 |
| NZ-794 | 0.06 | 0.155 | 0.345 | 39.229 | 2.5 |
| NZ-795 | 0.048 | 0.080 | 0.135 | >39.84 | 5 |
| NZ-796 | 0.25 | 1.945 | 6.913 | >39.84 | |
| NZ-797 | 0.049 | 0.062 | 0.111 | >39.84 | 2.5 |
| NZ-798 | 0.3 | 1.551 | 1.119 | >39.84 | |
| NZ-799 | 1.24 | 2.542 | 6.584 | >39.84 | |
| NZ-800 | 0.101 | 0.204 | 4.913 | >39.84 | |
| NZ-801 | 0.056 | 0.042 | 0.081 | >39.84 | |
| NZ-802 | 0.067 | 0.060 | 0.041 | >39.84 | |
| NZ-803 | 0.091 | 0.183 | 0.325 | >39.84 | |
| NZ-804 | 0.03 | 0.015 | 0.162 | >39.84 | |
| NZ-805 | 0.048 | 0.058 | 0.064 | >39.84 | |
| NZ-806 | 18.87 | >9.61 | >9.61 | >39.84 | |
| NZ-807 | 0.61 | 1.990 | >9.61 | >39.84 | |
| NZ-808 | 0.032 | 0.025 | 0.030 | >39.84 | |
| NZ-809 | 0.043 | 0.088 | 0.111 | >39.84 | |
| NZ-810 | 0.075 | 0.482 | 0.434 | >39.84 | |
| NZ-811 | NA | >9.61 | >9.61 | >39.84 | |
| NZ-812 | 0.55 | | | | |
| NZ-813 | NA | | | | |
| NZ-814 | 0.053 | | | | |
| NZ-815 | 0.03 | | | | |
| NZ-816 | 0.03 | | | | |
| NZ-817 | 0.033 | | | | |
| NZ-818 | NA | | | | |
| NZ-819 | 20 | | | | |
| NZ-820 | >80 | | | | |
| NZ-821 | 0.044 | | | | |
| NZ-822 | NA | | | | |
| NZ-823 | NA | | | | |
| ZS-312 | 0.44 | 1.04 | >9.61 | >39.84 | |
| ZS-313 | 8.2 | >9.61 | >9.61 | >39.84 | |
| ZS-316 | 0.14 | 0.7 | >9.61 | >39.84 | 2.5 |
| ZS-317 | 0.18 | 0.9 | >9.61 | >39.84 | |
| ZS-318 | 0.15 | 0.7 | >9.61 | >39.84 | |
| ZS-319 | 0.21 | 0.92 | >9.61 | 32.1 | |
| ZS-320 | 1.18 | 2.53 | >9.61 | >39.84 | |
| ZS-324 | 0.54 | 2.79 | >9.61 | 21.08 | |
| ZS-325 | 1.51 | 5.68 | >9.61 | >39.84 | |
| ZS-329 | 0.089 | 0.33 | >9.61 | 21.29 | 5 |
| ZS-331 | 0.37 | | | | |
| ZS-333 | NA | >9.61 | >9.61 | >39.84 | |
| ZS-335 | NA | >9.61 | >9.61 | >39.84 | |
| ZS-336 | 0.054 | 0.252 | 0.314 | 31.507 | 2.5 |
| ZS-337 | NA | >9.606 | >9.606 | >39.84 | |
| ZS-338 | >1 | 7.073 | >9.606 | >39.84 | |
| ZS-339 | 1.15 | 4.059 | >9.61 | >39.84 | |
| ZS-342 | 0.15 | 0.406 | 1.182 | >39.84 | 5 |
| ZS-343 | 9.5 | >9.61 | >9.61 | >39.84 | |
| ZS-344 | NA | >9.61 | >9.61 | >39.84 | |
| ZS-345 | NA | >9.61 | >9.61 | >39.84 | |
| ZS-346 | 0.27 | 1.069 | 2.036 | >39.84 | |
| ZS-347 | 1.7 | 4.251 | >9.61 | >39.84 | |
| ZS-355 | 0.053 | 0.223 | 0.377 | >39.84 | 20 |
| ZS-361 | 0.044 | 1.180 | 4.320 | 34.249 | 20 |
| PKJ-595 | NA | | | | |
| PKJ-609 | NA | | | | |
| PKJ-612 | NA | | | | |
| PKJ-606 | NA | | | | |
| PKJ-607 | NA | | | | |
| PKJ-663 | >80 | | | | |
| PKJ-703 | 0.93 | | | | |
| PKJ-704 | 1.6 | | | | |
| PKJ-710 | NA | | | | |
| PKJ-683 | NA | | | | |
| PKJ-672 | 0.76 | | | | |

TABLE 3
The activity of select example compounds against mpro.

| | Enzyme IC50 | CoV-2 HeLa EC50 |
|---|---|---|
| Name | [uM] | [uM] |
| AA-625 | 0.004 | 0.072 |
| AA-663 | 0.014 | 0.063 |
| MKP-1290 | 0.096 | 0.105 |
| MKP-1355 | 0.012 | 0.094 |
| MKP-1371 | 0.013 | 0.047 |
| NZ-795 | 0.048 | 0.080 |
| NZ-801 | 0.012 | 0.042 |
| NZ-804 | 0.009 | 0.015 |
| NZ-805 | 0.014 | 0.058 |
| NZ-808 | 0.010 | 0.025 |
| NZ-809 | 0.015 | 0.088 |
| NZ-815 | 0.030 | 0.097 |
| NZ-816 | 0.017 | 0.040 |
| NZ-821 | 0.044 | 0.023 |
| NZ-822 | 0.054 | 0.089 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound defined by Formula (I) below. 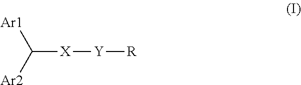
or a pharmaceutically acceptable salt or prodrug thereof, wherein, as valence and stability permit,
Ar1 and Ar2 are independently selected from aryl or heteroaryl, each optionally substituted with one or more substituents individually chosen from R⁵;
X is chosen from the following groups: 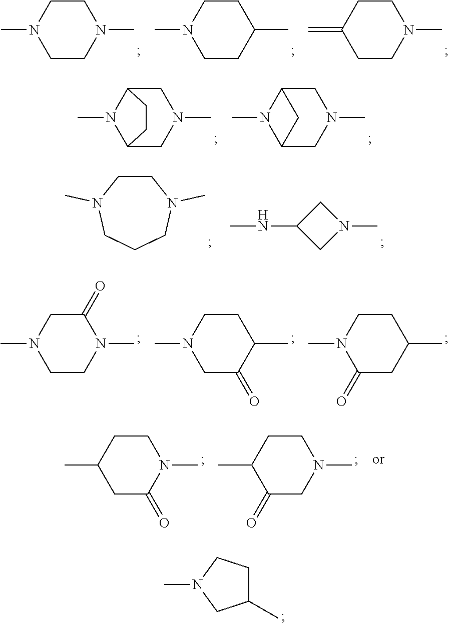
Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

2. The compound of claim 1, wherein Ar1 and Ar2 are each independently a 5-7 membered aryl or heteroaryl group, each optionally substituted with one or more substituents individually chosen from R⁵.

3. The compound of claim 1, wherein Ar1 and Ar2 are each independently chosen from phenyl and pyridine, each optionally substituted with one or more substituents individually chosen from R⁵.

4. The compound of claim 1, wherein X is chosen from the following groups:

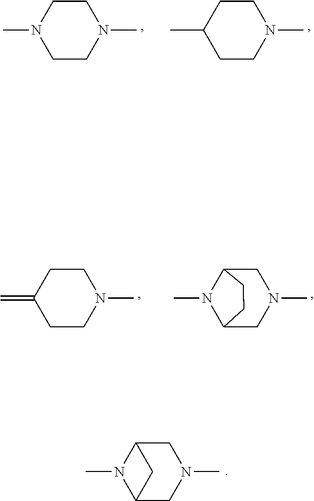

5. The compound of claim 1, wherein X is chosen from the following groups:

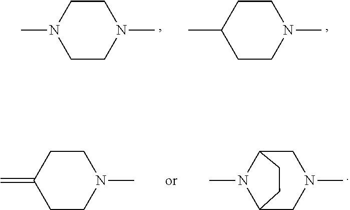

6. The compound of claim 1, wherein X is

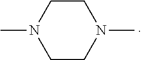

7. The compound of claim 1, wherein X is

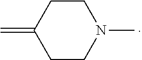

8. The compound of claim 1, wherein Y is —CO—.

9. The compound of claim 1, wherein R is a 5-10 membered ring (e.g., a 5-7 membered ring) optionally substituted with one or more substituents individually chosen from R⁵.

10. The compound of claim 1, wherein R is chosen from a phenyl, pyridine, thiophen, furan, pyrrole, imidazole, thiazole, oxazole, pyrimidine, pyrazine, indole, benzothiophene, benzofuran, benzoxazole, benzothiozole, benzimidazole, piperazine, piperidine, morpholine, quinuclidine, pyrrolo-pyridine, imidazo-pyridine, pyrazolo-pyridine, furo-pyridine and thieno-pyridine, or pyrrolidine group each optionally substituted with one or more substituents individually chosen from R⁵.

11. The compound of claim 1, wherein R is a pyrrlo-pyridine or azaindole group optionally substituted with one or more substituents individually chosen from R⁵.

12. A compound defined by Formula (II) below 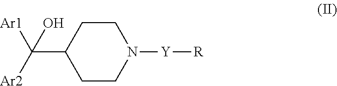
or a pharmaceutically acceptable salt or prodrug thereof, wherein, as valence and stability permit,
Ar1 and Ar2 are independently selected from aryl or heteroaryl, each optionally substituted with one or more substituents individually chosen from R⁵;
Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

13-18. (canceled)

19. A compound defined by any of Formula (III)—(XI) 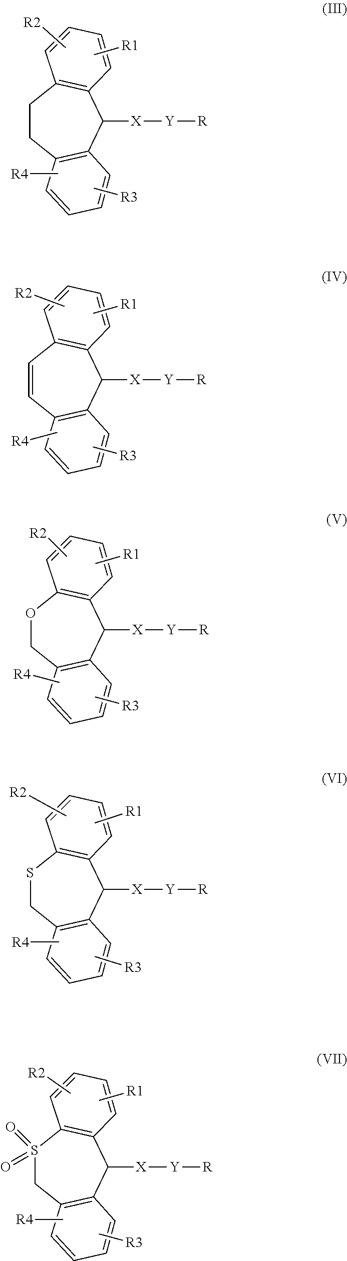 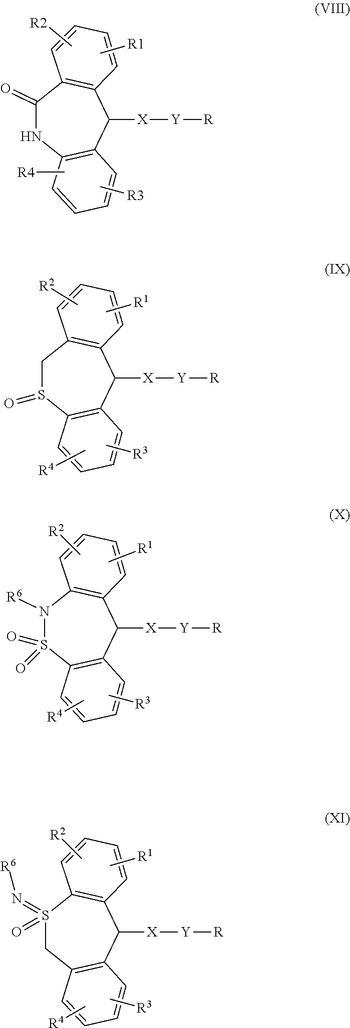
pharmaceutically acceptable salts or prodrugs thereof, wherein, as valence and stability permit,
X is chosen from the following groups: 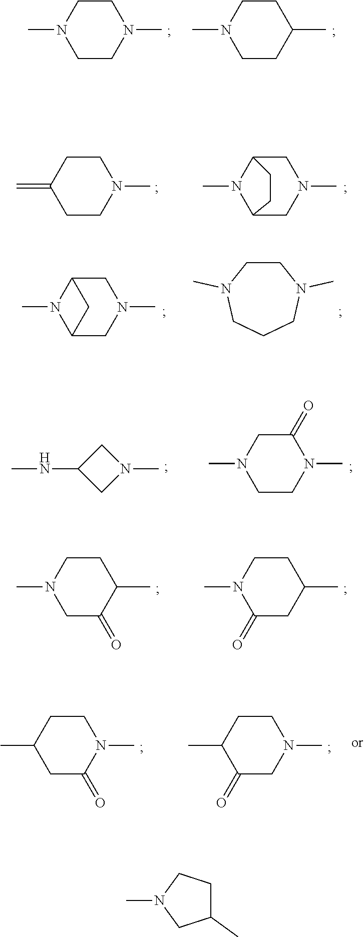
Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵;
R¹, R², R³, and R⁴ are each individually chosen from hydrogen, hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, and heterodialkylaminocarbonyl;
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

20-26. (canceled)

27. A compound defined by Formula (XII) below 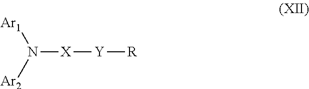
or a pharmaceutically acceptable salt or prodrug thereof, wherein, as valence and stability permit,
Ar1 and Ar2 are independently selected from aryl or heteroaryl, each optionally substituted with one or more substituents individually chosen from R⁵;
X is chosen from the following groups: 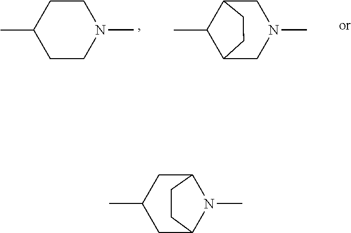
Y is selected from —CO—, —SO₂—, —S(═O)—, —S(O)₂NR⁶—, —S(═O)(=NR⁶)—, and —CH₂—;
R is selected from aryl, heteroaryl, cycloalkyl, and cycloheteroalkyl, each optionally substituted with one or more substituents individually chosen from R⁵; and
R⁵ is chosen from hydroxy, halogen, —CN, —NO₂, amino, alkylamino, dialkylamino, alkyl, haloalkyl; alkylthio; haloalkylthio; alkoxy, haloalkoxy, alkenyl, haloalkenyl, alkynyl, haloalkynyl, alkylsulfinyl, haloalkylsulfinyl, alkylsulfonyl, haloalkylsulfonyl, alkylcarbonyl, haloalkylcarbonyl, alkoxycarbonyl, haloalkoxycarbonyl, alkylaminocarbonyl, heteroalkylaminocarbonyl, dialkylaminocarbonyl, heterodialkylaminocarbonyl, sulfonamido, and sulfoximino; and
R⁶ is chosen from hydrogen, alkyl, haloalkyl, alkenyl, haloalkenyl, alkynyl, haloalkynyl, aryl, heteroaryl, cycloalkyl, cycloalkenyl, heterocycloalkyl, or heterocycloalkenyl group, each optionally substituted with one or more substituents individually chosen from R⁵.

28-33. (canceled)

34. The compound of claim 1, wherein the compound is described in Table 1.

35. The compound of claim 1, wherein the compound is 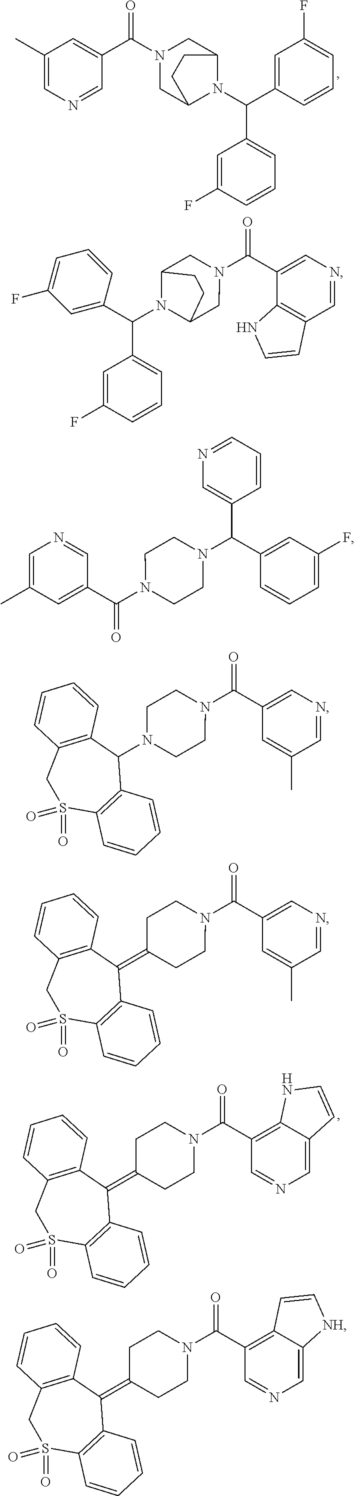 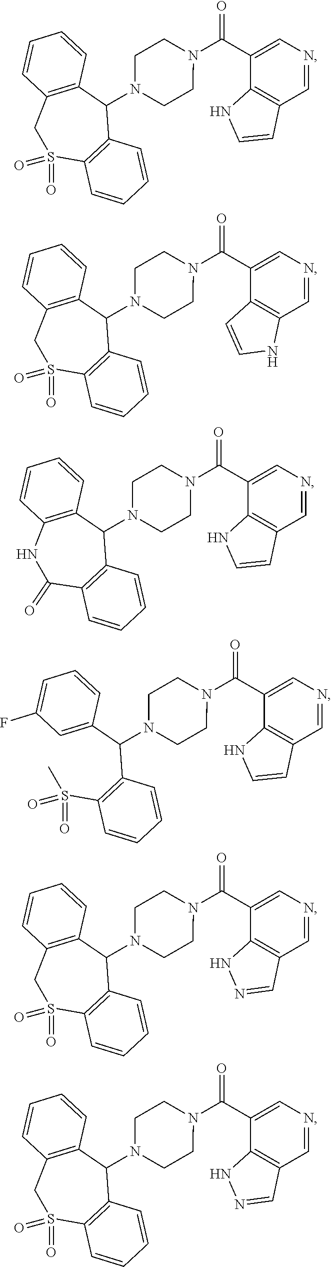 [SMILES: O=C(c1cncc2cc[nH]c12)N1CCN(C2c3ccccc3CS(=O)(=O)c3ccccc32)CC1 ; O=C(c1cncc2[nH]ccc12)N1CCN(C2c3ccccc3CS(=O)(=O)c3ccccc32)CC1 ; O=C1Nc2ccccc2C(N2CCN(C(=O)c3cncc4cc[nH]c34)CC2)c2ccccc21 ; CS(=O)(=O)c1ccccc1C(c1cccc(F)c1)N1CCN(C(=O)c2cncc3cc[nH]c23)CC1 ; O=C(c1cncc2cn[nH]c12)N1CCN(C2c3ccccc3CS(=O)(=O)c3ccccc32)CC1 ; O=C(c1cncc2cn[nH]c12)N1CCN(C2c3ccccc3CS(=O)(=O)c3ccccc32)CC1]
or a pharmaceutically acceptable salt or prodrug thereof.

36. A pharmaceutical composition comprising a therapeutically effective amount of a compound defined by claim 1.

37. The pharmaceutical composition of claim 36, wherein the composition further comprises a cytochrome P450 3A4 (CYP-3A4) inhibitor or ritonavir or a pharmaceutical acceptable salt, ester, solvate, prodrug, or a derivative thereof.

38. (canceled)

39. A method of treating or preventing a coronavirus infection in a subject, the method comprising administering a therapeutically effective amount of a compound defined by claim 1 to the subject.

40. (canceled)

41. A method of inhibiting a coronavirus main protease, the method comprising contacting the coronavirus with an effective amount of a compound defined by claim 1.

42. (canceled)
